(19) United States
(12) Patent Application Publication
(10) Pub. No.: US 2025/0302893 A1
(43) Pub. Date: Oct. 2, 2025

(54) TREATMENT OF METABOLIC SYNDROME AND ASSOCIATED MORBIDITIES USING INTESTINAL TH17 CELLS OR INTESTINAL TH17 CELL-DERIVED MOLECULES

(71) Applicant: THE TRUSTEES OF COLUMBIA UNIVERSITY IN THE CITY OF NEW YORK, New York, NY (US)

(72) Inventor: Ivaylo I. IVANOV, New York, NY (US)

(21) Appl. No.: 19/064,523

(22) Filed: Feb. 26, 2025

Related U.S. Application Data
(63) Continuation of application No. PCT/US2023/073018, filed on Aug. 28, 2023.
(60) Provisional application No. 63/401,339, filed on Aug. 26, 2022.

(51) Int. Cl. A61K 35/745; A61K 39/00; A61P 3/00; A61P 3/04; C07K 16/24

(52) U.S. Cl. CPC A61K 35/745; A61P 3/00; A61P 3/04; C07K 16/24; C07K 16/244; A61K 2039/505; C07K 2317/76

(57) ABSTRACT

Certain intestinal or commensal bacteria (produced naturally or in vitro) induce Th17 cells, leading to production of IL-17, reducing lipid absorption, and thereby counteracting metabolic syndrome, obesity, and associated morbidities, such as type-2 diabetes, cardiovascular disease, and NASH/NAFLD. Administration of those bacteria, or of Th17 cells induced by those bacteria, to a subject helps counteract metabolic syndrome and associated morbidities. Antagonists of intestinal CD36 or molecules decreasing intestinal CD36, e.g., IL-17, may also be used to reduce lipid absorption. Depletion of Faecalibacterium rodentium or members of the Erysipelotrichaceae family in the subject may provide a similar result. ILC3 or IL-22 blockade (alone or combined with Th17 cell administration or induction if not already present in the subject), may further provide a similar result, protecting against metabolic disease.

### CROSS-REFERENCE TO RELATED APPLICATION

This application is a continuation application of International Application No. PCT/US2023/073018, filed Aug. 28, 2023, which claims priority in U.S. Provisional Patent Application No. 63/401,339, filed on Aug. 26, 2022, the contents of each of which is incorporated herein by reference in its entirety.

### STATEMENT REGARDING FEDERALLY SPONSORED RESEARCH OR DEVELOPMENT

This invention was made with government support under DK098378, AI44808, AI163069, and AI146817 awarded by the National Institutes of Health. The government has certain rights in the invention.

### FIELD OF THE INVENTION

The invention relates to treatment of metabolic syndrome and associated morbidities through modulating the Th17 pathway in the intestines.

### BACKGROUND OF THE INVENTION

Obesity and metabolic syndrome are complex physiological conditions that lead to many pathologies, including cardiovascular disease, stroke, and type 2 diabetes (T2D). Dietary changes are a major factor for the increase in incidence of obesity and metabolic syndrome. In both humans and mice, western-style high-fat diet (HFD) initiates a cascade of events that ultimately result in obesity and obesity-associated metabolic complications, such as metabolic syndrome and T2D. Although much is known about later stage pathophysiology of these conditions, the initiating events are incompletely understood. In addition, the role of non-fat dietary components is not well-established. For example, whether sugar content in diets is a significant contributor to metabolic syndrome is debatable and the mechanisms by which sugar may drive metabolic disorders are unclear.

The intestine is the largest immune organ and interfaces dietary antigens with the host. The intestinal immune system has emerged as an important regulator of metabolic homeostasis.

HFD has been implicated in increasing intestinal inflammation, which can contribute to endotoxemia and adipose tissue inflammation. The mechanisms by which HFD increases intestinal inflammation, as well as the dietary components mediating these changes have not been defined. Moreover, how mucosal immune cells affect diet-induced obesity (DIO) and metabolic syndrome is unclear.

CD4 T cells play central role in maintaining tissue homeostasis and direct the nature of immune responses in the gut and other tissues. However, the contribution of individual T helper subsets to metabolic syndrome is less clear. Th17 cells can promote metabolic syndrome-associated inflammatory phenotypes, as well as protection from obesity and metabolic syndrome. Similarly, type 3 innate lymphoid cells (ILC3) and ILC3-derived IL-22 have been reported to exert both protective and promoting effects in metabolic syndrome. Therefore, the role of type 3 immune cells seems complex and possibly context-dependent; however, the nature of the environmental, or other, signals controlling this heterogeneity of function is not known.

Microbiota are important modulators of intestinal immunity, including T cell and ILC3 responses. HFD induces changes in microbiota composition that play crucial role in obesity phenotypes. How intestinal microbes regulate metabolic syndrome is incompletely understood. HFD microbiota can promote metabolic syndrome by increasing energy harvest, including calory extraction and intestinal lipid absorption, or by inducing intestinal epithelial barrier disruption and endotoxemia, leading to adipose tissue inflammation. HFD-associated microbiota can also affect metabolic syndrome by modulating immune responses. The dietary and microbiota entities that regulate host immunity in the context of metabolic syndrome, as well as the cellular and molecular mechanisms involved are not known.

### SUMMARY OF THE INVENTION

Disclosed herein are methods for treating or preventing one or more of metabolic syndrome, obesity, and associated morbidities in a subject suffering from or prone to metabolic syndrome, obesity, or any associated morbidity (for example, type-2 diabetes, cardiovascular disease, and non-alcoholic steatohepatitis or non-alcoholic fatty liver). In one aspect, the method comprising maintaining or increasing the levels of intestinal Th17 cells in the subject. In some implementations, the method further comprises blocking type 3 innate lymphoid cells (ILC3) or IL-22 in the subject.

In some embodiments, the levels of intestinal Th17 cells are maintained or increased by administering to the subject an effective amount of commensal bacteria that induces production of Th17 cells. in some aspects, the commensal bacteria comprise a species selected from the group consisting of: Bifidobacterium, Eggerthella, Muribaculum, Olsenella, and Ruminococcus. In other aspects, the commensal bacteria comprise Bifidobacterium psudolongum.

In other embodiments, the levels of intestinal Th17 cells are maintained or increased by administering to the subject an effective amount of commensal bacteria-induced Th17 cells.

In another aspect, the method comprises administering to the subject an effective amount of IL-17 or other intestinal Th17-cell derived molecules. In still other aspects, the method comprises altering the subject's intestinal microflora. For example, the method comprises depleting Faecalibacterium rodentium or its homologue (for example, Holdemanella biformis) in the subject's intestinal microflora. As another example, the method comprises depleting Erysipelotrichaceae in the subject's intestinal microflora. In yet other aspects, the method comprising a blockade of type 3 innate lymphoid cells (ILC3) or IL-22 in the subject.

Also disclosed are a method of manipulating dietary constraints or requirements in a subject based on levels of intestinal Th17 cells or Th17 cell function, e.g., IL-17. A method of decreasing lipid absorption in a subject is also disclosed. The method comprises administering to the subject an effective amount of antagonists of intestinal CD36, NPC1L1, or SCD-1.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-1O show that HFD disrupts intestinal immune homeostasis by eliminating Th17-inducing microbiota. FIGS. 1A-IF depict flow cytometry for T cell transcription factors (FIGS. 1A-1D) and cytokines (FIGS. 1E and 1F) in SI LP CD4 T cells in C57BL/6 mice fed NCD or HFD for 4 weeks. Plots gated on TCRβ⁺CD4⁺ cells. Treg, Foxp3⁺RORγt^{neg}; Th17, IL-17⁺IFNγ^{neg}. Data from two out of multiple independent experiments, N=7 mice/group. FIG. 1G depicts an exemplary quantitative RT-PCR of Il17a expression in terminal ileum of C57BL/6 mice fed NCD or HFD for 5 weeks. Data combined from three independent experiments, N=7-10 mice/group. FIG. 1H shows the Th1 cells (IFNγ⁺IL-17^{neg}) from experiments in FIG. 1E. FIGS. 1I and 1J show time course of the proportion of Th17 cells (RORγt⁺Foxp3^{neg}) in SI LP CD4 T cells (FIG. 1I) and segmented filamentous bacteria (SFB) levels in feces (FIG. 1J) in C57BL/6 mice fed NCD or HFD. Data from two out of multiple independent experiments N=3-8 mice/group. FIG. 1K shows an exemplary quantitative PCR for SFB 16S DNA in terminal ilcum mucosa of C57BL/6 mice fed NCD or HFD for 7 days. Data combined from two independent experiments, N=4 mice/group. FIG. 1L depicts the experimental scheme for FIGS. 1M-1O. CD45.2 C57BL/6 mice were colonized with SFB and switched to HFD a week later. A week later, the animals received SFB-specific 7B8/CD45.1/IL-17A-GFP TCR Tg CD4 T cells. Tg T cells were analyzed 8 days after transfer. FIGS. M-1O depict representative FACS plots (FIG. 1M) and statistics (FIGS. 1N and 1O) of expansion (FIG. 1N) and Th17 cell differentiation (FIG. 1O) of transferred Tg CD4 T cells in SI LP on Day 15. Data combined from two independent experiments, N=6 mice/group.

FIGS. 2A-2R show that microbiota-induced Th17 cells protect from metabolic syndrome. FIG. 2A depicts an exemplary quantitative PCR for SFB 16S DNA in feces from WT, STOP and STOP/CD4 mice fed NCD or HFD for indicated times. Data from two out of several independent experiments, N=3-5 mice/group. FIG. 2B show ILC3 in SI LP of WT, STOP and STOP/CD4 mice fed NCD or HFD for 5 weeks. Data from two out of multiple independent experiments, N=5-6 mice/group. FIGS. 2C and 2D show the percentage of SI LP Th17 cells (IL-17A⁺IFNγ^{neg}) in SFB-positive (FIG. 2C) and SFB-negative (FIG. 2D) STOP and STOP/CD4 mice on Day 40 of HFD feeding. Data from three (FIG. 2C) or two (FIG. 2D) independent experiments out of multiple experiments, N=6-7 mice/group. FIGS. 2E-2J depict metabolic analysis of SFB-negative (FIGS. 2E-2G) and SFB-positive (FIG. 2H-2J) mice of the indicated genotypes fed NCD or HFD for 4-5 weeks. SFB-positive mice were colonized with SFB by oral gavage two weeks prior to diet transition. FIGS. 2E and 2H show changes in body weight. FIGS. 2F, 2G, 2I, and 2J show insulin tolerance test on Day 28 of HFD. AOC, area over the curve. Data from two (FIGS. 2E-2G) or three (FIGS. 2H-2J) independent experiments out of several experiments, N=6-11 mice/group. FIGS. 2K and 2L show exemplary quantitative PCR for total bacterial 16S DNA (FIG. 2K) and quantitative RT-PCR for Tnfa transcripts in liver of STOP and STOP/CD4 mice fed HFD for 5 weeks. Data combined from two independent experiments, N=4-6 mice/group. FIGS. 2M and 2N show body weight change (FIG. 2M) and insulin tolerance test at Day 28 (FIG. 2N), of SFB-positive STOP/CD4 mice treated with IgG control or anti-CD4 neutralizing antibody and fed HFD for 5 weeks. Data combined from two independent experiments, N=5 mice/group. FIGS. 2O and 2P show body weight change (FIG. 2O) and insulin tolerance test at Day 28 (FIG. 2P), of HFD-fed SFB-positive STOP/CD4 and T cell-deficient STOP/CD4 mice (TCRβ^{-/-}/RORγ-STOP/CD4-Cre). Data combined from two independent experiments, N=7 mice/group. FIGS. 2Q and 2R show body weight change (FIG. 2Q) and insulin tolerance test at Day 28 (FIG. 2R), of HFD-fed SFB positive STOP mice with and without transfer of total WT CD4 T cells. Data combined from two independent experiments, N=5 mice/group. See also FIGS. 9A-9H and 10A-10R.

FIGS. 3A-3L show that probiotic Th17 cell-inducing bacteria ameliorate metabolic syndrome. FIG. 3A depicts the experimental scheme of probiotic treatment. FIG. 2B shows an exemplary quantitative PCR for SFB 16S DNA in feces on Day 28 of HFD. N=6-8 mice/group. FIG. 3C shows an exemplary quantitative RT-PCR of Il17a transcripts in terminal ilcum at 5 weeks on HFD. N=6 mice/group. FIGS. 3D-3F show flow cytometry of cytokines in S1LP CD4 T cells at 5 weeks on HFD. N=3 mice/group. FIGS. 3G-3L show metabolic analyses, including body weight change (FIGS. 3G and 3H), insulin tolerance test (FIGS. 31 and 3J), and oral glucose tolerance test (FIGS. 3K and 3L) at 4 weeks. N=8-11 mice/group. Data combined from two independent experiments with similar results. AOC, area over the curve. AUC, area under the curve. See also FIGS. 11A-11D.

FIGS. 4A-4S show that dietary sugar promotes metabolic syndrome through elimination of commensal Th17 cells. FIG. 4A shows SFB 16S DNA in feces from C57BL/6 mice fed natural ingredients normal chow diet (NCD) and various purified diets for 1 week. LFD, low-fat diet. HFD Inulin, HFD supplemented with inulin. Data combined from two independent experiments, N=3-11 mice/group. FIG. 4B shows SFB 16S DNA in feces from C57BL/6 mice fed NCD, HFD, or a 50:50 mix NCD:HFD. Data combined from two independent experiments, N=3-6 mice/group. FIG. 4C shows SFB 16S DNA in feces from C57BL/6 mice fed NCD plus various concentrations of sucrose (SUC) in the drinking water. Data from two out of multiple independent experiments, N=3-7 mice/group. FIGS. 4D-4G shows RORγt⁺ (FIG. 4D) Th17 cells, IL-17A⁺ (FIG. 4E) Th17 cells, and RORγt expression in Foxp3^{neg}TCRβ⁺CD4⁺ cells (FIGS. 4F and 4G) in SI LP of mice fed NCD with and without 10% sucrose (SUC) in the drinking water for 1 week. Data from two out of multiple independent experiments, N=6-7 mice/group. FIGS. 4H-4O show the metabolic and immune cell phenotypes of SFB-negative (FIGS. 4H-4K) or SFB-positive (FIGS. 4L-4O) C57BL/6 mice fed NCD, HFD, or sugar-free HFD (SF-HFD) for 4 weeks. SFB-positive mice were colonized with SFB by oral gavage two weeks prior to the change of diet. FIGS. 4H and 4L show changes in body weight. FIGS. 4I, 4J, 4M, and 4N depict insulin tolerance test on Day 28. FIGS. 4K and 4O show SI LP Th17 cells (IL-17A⁺IFNγ^{neg}) as proportion of TCRβ+CD4+ cells on Day 40. Data from two out of four independent experiments, N=5-9 mice/group. FIGS. 4P and 4Q show body weight change (FIG. 4P) and insulin tolerance test on Day 28 (FIG. 4Q), of SFB-colonized C57BL/6 mice fed NCD, HFD, SF-HFD, or SF-HFD supplemented with 10% sucrose (SUC) in the drinking water for 5 weeks. Data combined from two independent experiments, N=5 mice/group. FIGS. 4R and 4S show body weight change (FIG. 4R) and insulin tolerance test on Day 28 (FIG. 4S), of SFB-colonized Th17 cell-deficient RORγt^{flox/flox}/CD4-Cre mice and control littermates fed SF-HFD. Data combined from two independent experiments, N=5 mice/group. See also FIGS. 12A-12S.

FIGS. 5A-5K show that dietary sugar displaces Th17 microbiota by increasing Faecalibaculum rodentium (Frod). FIG. 5A show SFB 16S DNA in feces of germ-free mice monocolonized with SFB and fed NCD or NCD plus 10% sucrose (SUC) in the drinking water. Data from one out of two independent experiments, n=5 mice/group. FIG. 5B show an exemplary PCoA plot of 16S microbiota analysis in feces from WT mice on NCD, or 10 days on HFD or NCD plus 10% sucrose in drinking water. Data from one out of two independent experiments, N=4 mice/group. FIGS. 5C and 5D show the family level taxonomy and relative abundance (FIG. 5C) and OTU taxonomy and absolute abundance (FIG. 5D) in 16S analysis of microbiota in the groups in (FIG. 5B). FIGS. 5E and 5F show the enrichment analysis of absolute abundance of microbiota OTUs comparing (FIG. 5E) HFD vs NCD and (FIG. 5F) NCD+10% sucrose vs NCD. FIG. 5G shows exemplary quantitative PCR data for Frod in feces from WT mice on NCD, or 10 days on HFD or NCD plus 10% sucrose in drinking water. Data from one out of two independent experiments, N=4 mice/group. FIG. 5H shows the correlation of SFB and Frod levels in individual animals. Data from one out of two independent experiments, N=3-5 mice/group. FIGS. 51-5K show that sugar-mediated Frod expansion requires ILC3. FIG. 5I depicts the experimental scheme. WT and STOP/CD4 mice were pre-treated with Ampicillin (Amp) before introducing Frod by oral gavage and 10% sucrose in the drinking water. Frod levels in feces of (FIG. 5J) WT or (FIG. 5K) ILC3-deficient STOP/CD4 mice on Day 2 (D2) and Day 10 (D10) post gavage. Data combined from two independent experiments, N=6 mice/group. Sec also FIGS. 13A-130.

FIGS. 6A-6K show that Frod is sufficient to displace SFB. FIGS. 6A-6C show germ-free C57BL/6 mice were colonized with SFB, cither alone or together with Frod. FIG. 6A depicts the experimental scheme. SFB (FIG. 6B) and Frod (FIG. 6C) levels were followed in feces for 2 weeks. Data from one out of two independent experiments, N=5 mice/group. FIGS. 6D-6F depict results related to germ-free C57BL/6 mice colonized with SFB and Frod for 17 days before addition of 10% sucrose (SUC) in the drinking water. FIG. 6D shows the experimental scheme. SFB (FIG. 6E) and Frod (FIG. 6F) levels in feces 10 days after SUC introduction. N=4 mice/group. FIG. 6G-61 depict results related to germ-free C57BL/6 mice were colonized with SFB and 10 days later colonized with Frod. FIG. 6G show the experimental scheme. SFB (FIG. 6H) and Frod (FIG. 6I) levels were followed in feces for 17 days. Data from two out of four independent experiments, N=7-8 mice/group. FIG. 6J show results related to SFB-monocolonized mice were gavaged with Frod and bacteria levels were followed in feces. N=6-8 mice/group. FIG. 6K depict an exemplary transmission electron microscopy of terminal ileum at the 24-hour timepoint of FIG. 6J. SFB and Frod in the mucus (left) and lumen (right). See also FIGS. 13A-130.

FIGS. 7A-7Q show that commensal Th17 cells prevent metabolic syndrome by regulating intestinal lipid absorption. FIGS. 7A-7C show the total lipid contents in IEC (FIG. 7A), liver (FIG. 7B) and feces (FIG. 7C) of STOP and STOP CD4 mice fed HFD for 5 weeks. Data from two independent experiments, N=4 mice/group. FIG. 7D depicts Cd36 transcripts in ileum IEC from STOP and STOP/CD4 mice fed HFD for 4 weeks. Data from two independent experiments, N=3-5 mice/group. FIG. 7E shows Cd36 transcripts in ilcum IEC from STOP/CD4 mice and TCRβ-KO STOP/CD4 mice (DKO) fed HFD for 5 weeks. Data from two independent experiments, N=3-5 mice/group. FIGS. 7F-7H depict Cd36 transcripts in IEC from duodenum (FIG. 7F), jejunum (FIG. 7G) and ileum (FIG. 7H) of WT mice and Th17 cell-deficient RORγt^{flox}/CD4-Cre mice under NCD. Data from two independent experiments, N=4 mice/group. FIG. 7I depicts Cd36 transcripts in ileum IEC of IL-17A-deficient mice and corresponding WT littermates. Data from two independent experiments, N=5-8 mice/group. FIG. 7J depicts Cd36 transcripts in ileum IEC of WT mice treated with anti-IL-17A neutralizing or control IgG antibody. Data from two independent experiments, N=4-6 mice/group. FIG. 7K depicts Cd36 transcripts in terminal ileum enteroids treated with rIL-17A in vitro (analysis of RNA-Seq data from Kumar et al., 2016). FIG. 7L depicts Cd36 transcripts in ileum IEC from SFB-negative and SFB-positive WT mice fed NCD, HFD and SF-HFD for 10 days. Data combined from two independent experiments, N=3-5 mice/group. FIG. 7M-7P show the body weight change (FIGS. 7L and 7M) and insulin tolerance test on Day 28 (FIGS. 7N and 70) of WT and Cd36^{-/-}mice fed SF-HFD for 4 weeks. Data from one out of two independent experiments, N=4 mice/group. FIG. 7Q shows IL-17+Th17 cells in the SI LP of WT and Cd36^{-/-}mice fed SF-HFD for 5 weeks. Proportion of TCRβ+CD4⁺ cells. Data from one out of two independent experiments, N=4 mice/group. See also FIGS. 14A-140.

FIGS. 8A-8S show that HFD disrupts intestinal immune homeostasis by eliminating Th17-inducing microbiota. The results relate to FIGS. 1A-10. FIGS. 8A-8C show metabolic analyses at 5 weeks on high-fat diet (HFD) vs normal chow (NCD). Data from two out of multiple independent experiments. FIGS. 8D-8G show exemplary flow cytometry of SI LP CD4 T cells in WT C57BL/6 mice fed with NCD or HFD for 4 weeks. Plots gated on TCRβ+CD4+ lymphocytes. Data from two out of multiple independent experiments, N=7 mice/group. FIGS. 8H-8L show exemplary flow cytometry analysis of ILC3 (Lin^{neg}RORγt⁺) and ILC3 subsets in SI LP of WT C57BL/6 mice fed NCD or HFD for 5 weeks. Data from two out of multiple independent experiments, N=5-11 mice/group. FIGS. 8M and 8N show exemplary flow cytometry analysis of macrophage (Mf) (FIG. 8M) and T cell subsets (FIG. 8N) in visceral adipose tissue (AT) of WT C57BL/6 mice fed NCD or HFD for 4 weeks. Plots gated on CD45+ hematopoietic cells. Data from one experiment, N=7 mice/group. FIG. 8O show the time course of the proportion of Th1 cells within SI LP CD4 T cells in WT C57BL/6 mice fed HFD. Data combined from two independent experiments, N=4-8 mice/group. FIG. 8P shows SFB levels in feces of RORγt-flox/CD4-Cre (Th17 cKO) mice before (DO) and 7 days after (D7) transition to HFD. Data from one out of two independent experiments, N=3-4 mice/group. FIG. 8Q show the proportion of individuals showing high depletion in 20 Th17-inducing gut strains (Atarashi et al., 2015) in metabolic syndrome (N=163) versus control group (N=132) (metagenomic data from Pedersen et al., 2016). FIGS. 8R and 8S show the relative abundance of previously reported Th17-inducing gut strains, (R) Bifidobacterium adolescentis and(S) Eggerthella lenta in metabolic syndrome (N=163) versus control group (N=132) (metagenomic data from Pedersen et al., 2016).

FIGS. 9A-9G show that generation of ILC3-deficient, T cell-sufficient mice. The results relate to FIGS. 2A-2R. FIG. 9A depicts scheme of genetic modifications for generation of RORγ-STOP mice. FIGS. 9B and 9C show recovery of thymocyte development in RORγ-STOP/CD4-Cre (STOP/CD4) mice. FIG. 9C relates to double-positive (DP) thymocytes. Data from one out of multiple independent experiments, N=5 mice/group. FIGS. 9D and 9E show recovery of small intestinal RORγ⁺Th17 cells (FIG. 9E) and RORγ⁺Foxp3⁺ Tregs in STOP/CD4 mice. Plots gated on TCRβ+CD4+ cells. Data from two out of multiple independent experiments, N=5-7 mice/group. FIGS. 9F and 9G show lack of small intestinal ILC3 in STOP/CD4 mice. Plots in FIG. 9F gated on Lin^{neg} cells (TCRβ^{neg}CD3^{neg}B220^{neg}). Data from two out of multiple independent experiments, N=5-7 mice/group.

FIGS. 10A-10R show that microbiota-induced Th17 cells protect from metabolic syndrome. The results relate to FIGS. 2A-2R. FIG. 10A show exemplary quantitative RT-PCR for Il17a transcripts in terminal ileum tissue samples from WT, STOP and STOP/CD4 mice fed NCD or HFD for 5 weeks. Data combined from two independent experiments, N=6-8 mice/group. FIGS. 10B and 10C show glucose tolerance test of SFB-negative (FIG. 10B) and SFB-positive (FIG. 10C) WT, STOP and STOP/CD4 mice after 5 weeks on NCD or HFD. Data from two (FIG. 10B) and three (FIG. 10C) out of multiple independent experiments, N=5-10 mice/group. FIGS. 10D-10F show adiposity in epididymal fat (FIG. 10D), mesenteric fat (FIG. 10E), and brown adipose (FIG. 10F) tissue of WT mice on NCD, of HFD-fed WT, STOP, or STOP/CD4 mice. Data from one out of two independent experiments, N=3 mice/group. FIG. 10G show the daily food intake in HFD-fed WT, STOP, and STOP/CD4 mice. Data from one out of two independent experiments, N=4 mice/group. FIG. 10H shows exemplary quantitative RT-PCR for Ifng transcripts in terminal ileum samples from WT, STOP and STOP/CD4 mice fed HFD for 5 weeks. Data combined from two independent experiments, N=3-4 mice/group. FIGS. 10I and 10J show metabolic analyses at 8 weeks of SFB-positive HFD-fed STOP and STOP/CD4 mice. One experiment, N=5 mice/group. FIGS. 10K-10M show CD4 T cells in SI LP (FIG. 10K), SFB levels in feces (FIG. 10L) and glucose tolerance test (FIG. 10M) of STOP/CD4 mice treated with anti-CD4 mAb to deplete CD4 T cells or isotype control (IgG) and fed HFD for 5 weeks. Data combined from two independent experiments, N=3-5 mice/group. FIGS. 10N and 10O show glucose tolerance test (FIG. 10N) and SFB levels in feces (FIG. 10O) of STOP/CD4 and STOP/CD4 TCRβ-deficient double knock-out (DKO) mice fed HFD for 5 weeks. Data from two independent experiments, N=7 mice/group. FIGS. 10P-10R relate to transfer of WT CD4 T cells in ILC3/Th17-deficient STOP mice. FIG. 10P depicts the experimental design. FIGS. 10Q and 10R respectively show SFB levels in feces and transferred Th17 cells in SI LP five weeks after transfer. Data from one out of two independent experiments, N=5 mice/group. AUC, area under curve.

FIGS. 11A-11D show that probiotic Th17 cell-inducing bacteria ameliorate metabolic syndrome. The results relate to FIGS. 3A-3L. Quantitative RT-PCR of Ifng, Tnfa, Lipocalin (Lcn2), and Cxcl1 transcripts in terminal ileum at 4 weeks are respectively shown in FIGS. 11A-11D. Data from two independent experiments, N=6 mice/group.

FIGS. 12A-12S show that dietary sugar promotes metabolic syndrome through elimination of commensal Th17 cells. The results relate to FIGS. 4A-4S. FIG. 12A shows the correlation between sucrose content in various diets and fecal SFB levels in WT C57BL/6 mice after 1 week on the corresponding diet. FIG. 12B show exemplary quantitative PCR of SFB levels in terminal ileum mucosa of WT C57BL/6 mice fed natural gradient normal chow diet (NCD) or NCD+10% sucrose in the drinking water (SUC) for 1 week. Data from one out of two independent experiments, N=3 mice/group. FIG. 12C show exemplary quantitative PCR of SFB in feces of WT C57BL/6 mice fed NCD or NCD plus various sugars for one week. SUC, sucrose; MDX, maltodextrin; GAL, galactose. All sugars were provided at 10% w/v in the drinking water. Data from two independent experiments, N=3-7 mice/group. FIGS. 12D-12F show RORγt⁺ (FIG. 12D) RORγt^{neg} (FIG. 12E) Foxp3⁺ Tregs and IFNγ⁺ Th1 cells (FIG. 12F) in SI LP of mice fed NCD or NCD plus 10% sucrose in the drinking water (SUC) for 1 week. Data from two out of multiple independent experiments, N=6-7 mice/group. FIG. 12G show the relative abundance of previously reported Th17-inducing gut strains, Bifidobacterium adolescentis and Eggerthella lenta in shotgun metagenomic sequencing data (Johnson et al., 2019) from healthy volunteers with low or high sugar consumption (details in STAR Methods). FIG. 12H show the oral glucose tolerance test (OGTT) on Day 35 in SFB-negative (top) or SFB-positive (bottom) WT C57BL/6 mice fed NCD, HFD, or sugar-free HFD (SF-HFD). Data from two independent experiments, N=5-7 mice/group. FIG. 12I shows Th1 cells in SI LP of SFB-positive WT C57BL/6 mice fed NCD, HFD, or sugar-free HFD (SF-HFD). Data represented as proportion of TCRβ⁺CD4⁺ cells on Day 40. Data from two out of multiple independent experiments, N=5-9 mice/group. FIGS. 12J and 12K show exemplary quantitative PCR for total bacterial 16S DNA (L) and quantitative RT-PCR for Tnfa transcripts in liver of SFB-positive WT C57BL/6 mice fed NCD, HFD, or sugar-free HFD (SF-HFD) for 5 weeks. Data from two independent experiments, N=4-6 mice/group. FIGS. 12L and 12M show the metabolic phenotypes of SFB-negative or SFB-colonized C57BL/6 mice fed indicated diets for 9 weeks. Data from one experiment, N=5 mice/group. FIG. 12N show Th17 cells in SI LP of SFB-positive WT C57BL/6 mice fed NCD, HFD, SF-HFD, or SF-HFD supplemented with 10% sucrose (+SUC) in the drinking water for 5 weeks. Data from two independent experiments, N=3-5 mice/group. FIG. 12O show the oral glucose tolerance test (OGTT) of WT C57BL/6 mice fed NCD, HFD, SF-HFD, or SF-HFD supplemented with 10% sucrose (+SUC) in the drinking water for 5 weeks. Data from two independent experiments, N=3-5 mice/group. FIGS. 12P and 12Q show Th17 cells (RORγt⁺ CD4 T cells) (FIG. 12P) and oral glucose tolerance test (FIG. 12Q) of SFB-colonized Th17 cell-deficient RORγt^{flox/flox}, CD4-Cre mice and control littermates fed SF-HFD for 5 weeks. Data from one out of two independent experiments, N=3-5 mice/group. FIGS. 12R and 12S show exemplary quantitative PCR for total bacterial 16S DNA (FIG. 12R) and quantitative RT-PCR for Tnfa transcripts (S FIG. 12) in liver of SFB-colonized Th17 cell-deficient RORγt^{flox/flox}, CD4-Cre mice and control littermates fed SF-HFD for 5 weeks. Data from two independent experiments, N=5 mice/group.

FIGS. 13A-13O show that dietary sugar displaces Th17 microbiota by increasing Frod. The results relate to FIGS. 5A-5K and 6A-6K. FIG. 13A shows exemplary quantitative PCR of SFB in feces of WT and ILC3-deficient/Th17-sufficient STOP/CD4 mice before and 7 days after treatment with 10% sucrose (SUC) in the drinking water. Data from two independent experiments, N=6 mice/group. FIG. 13AB shows exemplary quantitative PCR of Frod in feces of WT mice on NCD or NCD plus various concentration of sucrose (SUC) in drinking water. Data plotted as relative fold change (FC) over NCD group. Data from one experiment, N=3 mice/group. FIG. 13C shows quantitative PCR of Frod in feces of WT mice fed NCD, HFD, LFD and HFD+Inulin for 1 week. Data plotted as relative fold change (FC) over NCD group. Data from one out of two independent experiments, N=3 mice/group. FIG. 13D shows exemplary quantitative PCR data of Frod in feces of WT mice on NCD or NCD plus various types of sugars for one week. SUC, sucrose; MDX, maltodextrin; GAL, galactose. All sugars were provided at 10% w/v in the drinking water. Data plotted as relative fold change (FC) over NCD group. Data from two independent experiments, N=3-7 mice/group. FIG. 13E shows exemplary quantitative PCR of Frod in feces of SFB-negative WT mice fed indicated diets for 1 week. Data from one out of two independent experiments, N=4 mice/group. FIG. 13F shows the kinetics of SFB and Frod in feces of WT mice on NCD following addition of 10% sucrose in the drinking water. Data plotted as relative fold change (FC) over NCD group. Data from one experiment, N=3 mice/group. FIG. 13G show exemplary quantitative PCR of Frod in feces of SFB-positive WT and ILC3-deficient/Th17-sufficient STOP/CD4 mice before and 7 days after treatment with 10% sucrose (SUC) in the drinking water. Data plotted as relative fold change (FC) over WT No sucrose group. Data from two independent experiments, N=6 mice/group. FIGS. 13H-13K show that Frod is sufficient to displace SFB. FIG. 13H depicts the experiment scheme. Germ-free C57BL/6 mice were colonized by oral gavage with SFB and a week later with cither Frod or Bifidobacterium pseudolongum (Bpl). FIG. 13I-13K follows the levels of the three microbes in feces by quantitative PCR for two weeks. Data from one experiment, N=3 mice/group. FIGS. 13L and 13M show RORγt⁺Foxp3^{neg} (L) and IL-17⁺ (M) Th17 cells in the SI LP of GF animals colonized first with SFB and then with or without Frod as per the experimental scheme on FIG. 6G. Mice were analyzed 7 days post Frod gavage. Data presented as proportion of TCRβ⁺CD4⁺ cells. Data from one out of two independent experiments, N=2-3 mice/group. FIGS. 13N and 13O show RORγt⁺Foxp3^{neg} (FIG. 13N) and IL-17⁺ (FIG. 13O) Th17 cells in the SI LP of gnotobiotic mice colonized with SFB and other bacteria. All animals were first colonized with SFB, followed by gavage with Frod or Bpl a week later as per the scheme on FIG. 13H. Mice were analyzed 7 days post Frod/Bpl gavage. Data presented as proportion of TCRβ⁺CD4⁺ cells. Data from one experiment, N=3 mice/group.

FIGS. 14A-140 show that commensal Th17 cells prevent metabolic syndrome by regulating intestinal lipid absorption. The results relate to FIGS. 7A-7Q. FIGS. 14A-14D show exemplary quantitative RT-PCR for transcripts of lipid transporters in ileum IECs from SFB-positive Th17-deficient STOP and Th17-sufficient STOP/CD4 mice fed HFD for 5 weeks. Data from two independent experiments, N=3-5 mice/group. FIG. 14E show exemplary quantitative RT-PCR for Cd36 transcripts in jejunum IECs from SFB-positive STOP and STOP/CD4 mice fed HFD for 5 weeks. Data from two independent experiments, N=3-5 mice/group. FIG. 14F show IL-22+CD4 T cells in the SI LP of STOP and STOP/CD4 mice fed HFD for 5 weeks. Data from two independent experiments, N=3-4 mice/group. FIG. 14G show exemplary quantitative RT-PCR of transcripts for the IL-22-induced IEC gene Reg3g in total ileum from STOP and STOP/CD4 mice fed HFD for 5 weeks. Data from two independent experiments, N=4 mice/group. FIG. 14H show quantitative RT-PCR for Cd36 transcripts in jejunum IECs from WT and IL-17A-deficient mice. Data from two independent experiments, N=5 mice/group. FIGS. 14I and 14J show IL-17A⁺ (FIG. 14I) and RORγt⁺ (FIG. 14J) Th17 cells in SI LP of SFB-positive IL-17A-deficient mice. Data from two independent experiments, N=4-5 mice/group. FIGS. 14K and 14L relate to small intestinal enteroids were treated with rIL-17A in vitro (analysis of scRNA-Seq data from Biton et al., 2018). FIG. 14O shows the ordination of profiled single cells by UMAP. FIG. 14L shows the expression level of Cd36 in the enterocyte cluster under different conditions. Statistics (L), Mann-Whitney U test. FIG. 14M show the total neutral lipid contents in feces of SFB-negative WT C57BL/6 mice fed NCD or HFD for 4 weeks. Data from one experiment, N=3 mice/group. FIG. 14N show exemplary quantitative RT-PCR for Cd36 transcripts in IECs from duodenum (Duo), jejunum (Jej) and ileum (Ile) of SFB-negative WT C57BL/6 mice fed NCD or HFD for 5 weeks. Data from two independent experiments, N=4 mice/group. FIG. 14O shows exemplary quantitative RT-PCR for Cd36 transcripts in IECs from jejunum (Jej) of SFB-positive WT C57BL/6 mice fed with indicated diets for five weeks. Data from one experiment, N=4 mice/group.

FIG. 15 depicts a schematic illustrating the network of interactions between dietary components, microbiota, and microbiota-regulated immune functions. This network of interaction define a microbiota-dependent mechanism for immuno-pathogenicity of dietary sugar and highlight an elaborate interaction between diet, microbiota, and intestinal immunity in regulation of metabolic disorders.

FIGS. 16A-16G show that sucrose disrupts the maintenance of SFB Th17 cells by disrupting microbial ecology. FIG. 16A demonstrates that sugar-free high fat diet (SF-HFD) depletes Th17 cell-inducing SFB. FIG. 16B shows that there is no remaining SFB antigen following SF-HFD. Lack of proliferation of exogenous SFB-specific 7B8 CD4 T cells following adoptive transfer into SF-HFD fed mice. FIGS. 16C and 16D show that SFB-specific (7B8) Th17 cells are maintained in the absence of SFB and SFB antigens in SF-HFD-fed, but not HFD-fed, mice. FIG. 16E shows that maintenance of SFB Th17 cells in SF-HFD is microbiota-dependent. Ampicillin treatment (Amp) depletes SFB Th17 cells in SF-HFD-fed mice. Fecal microbiota transplantation (FMT) preserves SFB Th17 cells in antibiotic-treated animals fed SF-HFD. FIG. 16F shows the microbiota species increased in SF-HFD but not HFD-fed mice. These species are candidates for maintaining or enhancing SFB Th17 cells. FIG. 16G demonstrates that Bifidobacterium pseudolongum (Bp) maintains SFB Th17 cells in antibiotic-treated mice fed SF-HFD.

### DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

The intestine is the largest immune organ and interfaces dietary antigens with the host. The intestinal immune system has emerged as an important regulator of metabolic homeostasis. HFD has been implicated in increasing intestinal inflammation, which can contribute to endotoxemia and adipose tissue inflammation. The mechanisms by which HFD increases intestinal inflammation, as well as the dietary components mediating these changes have not been defined. Moreover, how mucosal immune cells affect DIO and metabolic syndrome is unclear.

Inflammatory changes in the intestine precede liver and adipose tissue inflammation, which drive pathology in metabolic diseases such as metabolic syndrome and T2D (Tilg et al., 2020; Winer et al., 2016). Imbalance of intestinal immune homeostasis is an important initial step in the pathogenesis of these systemic conditions (Kawano et al., 2016; Luck et al., 2015).

CD4 T cells play central role in maintaining tissue homeostasis and direct the nature of immune responses in the gut and other tissues. However, the contribution of individual T helper subsets to metabolic syndrome is less clear. Th17 cells can promote metabolic syndrome-associated inflammatory phenotypes, as well as protection from obesity and metabolic syndrome. Similarly, type 3 innate lymphoid cells (ILC3) and ILC3-derived IL-22 have been reported to exert both protective and promoting effects in metabolic syndrome. Therefore, the role of type 3 immune cells seems complex and possibly context-dependent; however, the nature of the environmental, or other, signals controlling this heterogeneity of function is not known.

Microbiota are important modulators of intestinal immunity, including T cell and ILC3 responses. HFD induces changes in microbiota composition that play crucial role in obesity phenotypes. How intestinal microbes regulate metabolic syndrome is incompletely understood. HFD microbiota can promote metabolic syndrome by increasing energy harvest, including calory extraction and intestinal lipid absorption, or by inducing intestinal epithelial barrier disruption and endotoxemia, leading to adipose tissue inflammation. HFD-associated microbiota can also affect metabolic syndrome by modulating immune responses. The dietary and microbiota entities that regulate host immunity in the context of metabolic syndrome, as well as the cellular and molecular mechanisms involved are not known.

Disclosed herein are compositions and methods of preventing and/or treating obesity, metabolic syndrome, and associated morbidities (such as type-2 diabetes (T2D), cardiovascular disease, and non-alcoholic steatohepatitis or non-alcoholic fatty liver (NASH/NAFLD)) through modulation of Th17 pathway in the intestines. For the first time, microbiota-regulated intestinal immunity is shown to provide protection against obesity and metabolic syndrome and should therefore be considered critical therapeutic target in metabolic syndrome and T2D.

In one aspect, the method of preventing and/or treating obesity, metabolic syndrome, and associated morbidities comprises maintaining or increasing the levels of intestinal Th17 cells in a subject suffering from or prone to metabolic syndrome. In another aspect, the method comprises administering to the subject suffering from or prone to metabolic syndrome an effective amount of IL-17 or other intestinal Th17-cell derived molecules. In yet another aspect, the method comprises modulating the subject's intestinal microflora to favor the Th17 pathway. For example, the method comprises depleting of Faecalibacterium rodentium or its homologue in the subject's intestinal microflora or depleting Erysipelotrichaceae in the subject's intestinal microflora. In some aspects, the homologue of F. rodentium is Holdemanella biformis. In some implementations, the method comprises administering to the subject a composition comprising a species from Bifidobacterium, Eggerthella, Muribaculum, Olsenella, Ruminococcus. In particular implementations, the subject is administered a composition comprising Bifidobacterium pseudolongum. In still another aspect, the method comprises blocking type 3 innate lymphoid cells (ILC3) or IL-22 in the subject, for example through administering neutralizing antibodies targeting ILC3 or IL-22 to the subject.

In some implementations, the levels of intestinal Th17 cells in the subject are maintained or improved by administering to the subject an effective amount of commensal bacteria that induces production of Th17 cells. In some implementations, the commensal bacteria comprise a species from Bifidobacterium, Eggerthella, Muribaculum, Olsenella, Ruminococcus. In particular implementations, the subject is administered a composition comprising Bifidobacterium pseudolongum. In other embodiments, the subject is administered an antibiotic that that preserves or enhances the population of commensal Th17 cells. In some aspects, the antibiotic preserves or enhances the population of commensal Th17 cells while depleting segmented filamentous bacteria populations in the intestinal microflora. Such antibiotics include polymyxin B and streptomycin. In other embodiments, the levels of intestinal Th17 cells in the subject are maintained or improved by administering to the subject an effective amount of commensal bacteria-induced Th17 cells. These commensal Th17 cells could be isolated from a subject prior to the subject receiving an antibiotic treatment or dietary interventions and then expanded in vitro. The commensal Th17 cells could also be isolated from healthy donors and expanded in vitro. In some implementations, the commensal Th17 cells are generated in vitro.

The role of type 3 immunity in DIO and metabolic syndrome is complex. Pro-inflammatory Th17 cells are enriched in liver and adipose tissue of obese patients (Dalmas et al., 2014; Fabbrini et al., 2013). At the same time, intestinal Th17 cells have been proposed to provide protection (Garidou et al., 2015; Hong et al., 2017; Perez et al., 2019). Similarly, ILC3 and ILC3-derived IL-22 are considered guardians of the epithelial barrier and beneficial in metabolic syndrome (Wang et al., 2014; Zou et al., 2018). However, ILC3-derived IL-22 can also contribute to metabolic disease (Sasaki et al., 2019; Upadhyay et al., 2012; Wang et al., 2017). Results shown in the Examples help reconcile these seemingly contradicting reports and suggest that the role of ILC3 is context-dependent.

Using an ILC3-deficient model that allows for differentiation of Th17 cells, the Examples show that ILC3 provide protection from metabolic disease in the absence of SFB and SFB Th17 cells. This protection was relatively mild at the four-week timepoint examined but could be more significant long-term. The Examples also show that maintenance of commensal Th17 cells in ILC3-deficient mice confers lasting protection. Moreover, ILC3 function, likely through IL-22 production, was required for sugar-mediated expansion of Frod and consequent loss of SFB and protective Th17 cells. Therefore, ILC3 can counteract the protective role of Th17 cells and, in such context, contribute to the pathogenic effects of HFD. Thus, the effects of ILC3, and by extension IL-22, on complex phenotypes, such as metabolic syndrome, are dependent on microbiota composition and the presence of Th17 cells and this should be taken into consideration when interpreting experimental results or designing cytokine-based therapies.

As shown in the Examples, microbiota-controlled intestinal immunity, and in particular type 3 immunity, has a role in early induction of DIO and metabolic syndrome. Microbiota-induced Th17 cells are protective against DIO and metabolic syndrome. Specifically, intestinal microbiota protects against development of obesity, metabolic syndrome, and pre-diabetic phenotypes by inducing commensal-specific Th17 cells. These results suggest an alternative explanation for the pathogenic role of sugar in metabolic disease through suppression of immuno-protective microbiota.

High-fat, high-sugar diet promotes metabolic disease by depleting Th17-inducing microbes, and as shown in the Example, the recovery of intestinal or commensal Th17 cells restored protection. Microbiota-induced Th17 cells afforded protection by regulating lipid absorption across intestinal epithelium in an IL-17-dependent manner. Diet-induced loss of protective Th17 cells was mediated by the presence of sugar. Eliminating sugar from high-fat diet protected mice from obesity and metabolic syndrome in a manner dependent on intestinal or commensal-specific Th17 cells. Sugar and ILC3 promoted outgrowth of Faecalibaculum rodentium (Frod) that displaced Th17-inducing microbiota. These results define dietary and microbiota factors posing risk for metabolic syndrome, obesity, and associated morbidities, such as type-2 diabetes, cardiovascular disease, and non-alcoholic steatohepatitis or non-alcoholic fatty liver (NASH/NAFLD). They also define a microbiota-dependent mechanism for immuno-pathogenicity of dietary sugar and highlight an elaborate interaction between diet, microbiota, and intestinal immunity in regulation of metabolic disorders.

Thus, a network of interactions between dietary components, microbiota, and microbiota-regulated immune functions exists that collectively protect from or promote metabolic syndrome. The results also demonstrate that the effects of dietary modifications or effector cytokines on metabolic conditions are context-dependent and should be taken into consideration when evaluating therapeutic interventions.

Also disclosed are a method of manipulating dietary constraints or requirements in a subject based on levels of intestinal Th17 cells or Th17 cell function, e.g., IL-17.

A method of determining needed dietary constraints and requirement is also disclosed. In one aspect, the method comprises manipulating dietary constraints or requirements based on levels of intestinal Th17 cells or Th17 cell function (for example, IL-17 levels). In another aspect, the method comprises monitoring Th17 pathway activity in a subject and determining the subject is in need of reducing dietary sugar when the subject exhibits increased Th17 pathway activity. In some implementations, the Th17 pathway activity in the subject is monitoring by assessing the subject's intestinal microflora population.

As shown herein, commensal microbiota can protect from metabolic syndrome through modulation of intestinal T cell homeostasis. In particular, protective Th17 cells are commensal-specific and are depleted during DIO by diet-induced depletion of Th17-inducing microbiota. The Examples also show that sucrose as a dietary component is sufficient to deplete Th17-inducing bacteria and Th17 cells. While dietary sugar has been considered detrimental for metabolic disease, the underlying mechanisms are not well understood (Macdonald, 2016; Stanhope, 2016). Disclosed herein in is a conceptually distinct mechanism in which sucrose does not directly drive metabolic syndrome but counteracts the protective function of intestinal immune cells by modulating intestinal microbiota. Sucrose and fructose intake have been associated with increase in intestinal inflammation and inflammatory bowel disease (Laffin et al., 2019; Racine et al., 2016).

Dietary sugar can increase the inflammatory tone of the intestine indirectly by depleting intestinal microbes that maintain tissue homeostasis. Elimination of sugar from HFD protected mice from disease by preserving commensal Th17 cells. Importantly, SF-HFD exerted protection only in the presence of Th17 cell-inducing microbiota and provided no benefit in the absence of commensal Th17 cells. Therefore, dietary interventions may only provide benefit if appropriate microbiota-regulated immune mechanisms are also in place. It is expected that individual variations in such mechanisms will affect the success of diet-based therapies and should be taken into consideration.

As shown in the Examples, dietary sugar depletes SFB indirectly, by expanding other gut bacteria. Frod is one such microbe, and its expansion is sufficient to displace SFB and decrease SFB-induced Th17 cells. Frod colonizes the mucosal surface of ileum and colon (Zagato et al., 2020) and, as shown in the Examples, can be found in close proximity to SFB in gnotobiotic animals, suggesting that displacement could be mediated by direct interactions between the two species. This is also supported by the fact that Frod is present in low abundance in NCD-fed SPF mice without displacing SFB. SFB displacement required expansion of Frod by sugar or relatively large amounts of Frod in gnotobiotic animals, which suggests that an abundance threshold is required for Frod to displace SFB. The mechanisms by which Frod inhibits SFB will be important to investigate in the future. Thus, dietary effects on immunoregulatory microbes can be mediated by microbe-microbe interactions.

Dietary lipids are major drivers of the inflammatory effects of HFD, including barrier leakage, endotoxemia, and type 1 inflammation (Basson et al., 2020; Khan et al., 2021; Zmora et al., 2017). However, the detailed mechanisms involved and the relative contribution of these mechanisms to metabolic disease are not currently known. Commensal Th17 cells can decrease lipid absorption, and this will likely affect inflammatory phenotypes in the intestine and adipose tissue. Indeed, in most of the experiments in the Examples, the presence of commensal Th17 cells was accompanied by decrease in Th1 intestinal responses and bacterial translocation. Decrease in Th1 inflammation, including intestinal Th1 inflammation, improves obesity related metabolic phenotypes (Luck et al., 2015; Wong et al., 2011) and can contribute to the protective function of commensal Th17 cells. At the same time, intestinal or commensal Th17 cells may also influence low-grade inflammation independently of lipid absorption, for example by controlling local intestinal inflammation. Indeed, SFB-induced Th17 cells differ significantly from pathogen-induced inflammatory Th17 cells and may participate in maintenance of intestinal immune homeostasis (Khan et al., 2021; Omenetti et al., 2019; Wu et al., 2020). Therefore, intestinal or commensal Th17 cells may possess additional mechanisms of protection from metabolic disease.

CD36 is a critical regulator of lipid absorption and fat metabolism and CD36 deficiency is associated with resistance to obesity and metabolic syndrome (Cai et al., 2012; Febbraio et al., 1999; Hajri et al., 2007; Kennedy and Kashyap, 2011; Yang et al., 2018). Microbiota can promote host lipid absorption by enhancing epithelial CD36 (Wang et al., 2017). Microbiota can also restrain lipid absorption and prevent obesity by decreasing intestinal epithelial CD36 (Petersen et al., 2019). We find that commensal Th17 cells protect from DIO and metabolic syndrome by decreasing IEC expression of CD36 and intestinal lipid absorption in an IL-17-dependent manner. CD36 is expressed on multiple cell types and has pleiotropic roles in metabolic disease (Chen et al., 2022; Pepino et al., 2014). Whether Th17 cell mediated regulation of CD36 can protect through additional mechanisms requires further study.

Thus, further disclosed herein is a method of decreasing lipid absorption in a subject. The method comprises administering to the subject an effective amount of antagonists of intestinal CD36, NPC1L1, or SCD-1.

### EXAMPLES

The invention is further illustrated by the following examples that should not be construed as limiting. The contents of all references, patents, and published patent applications cited throughout this application, as well as the Figures, are incorporated herein by reference in their entirety for all purposes.

### Example 1. HFD Disrupts Intestinal Immune Homeostasis by Eliminating Th17-Inducing Microbiota

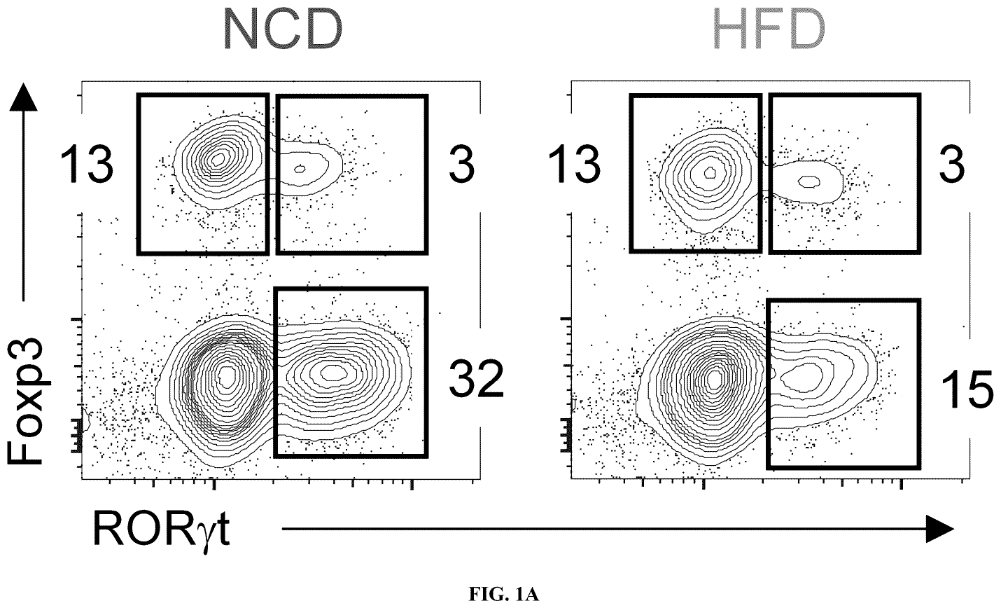

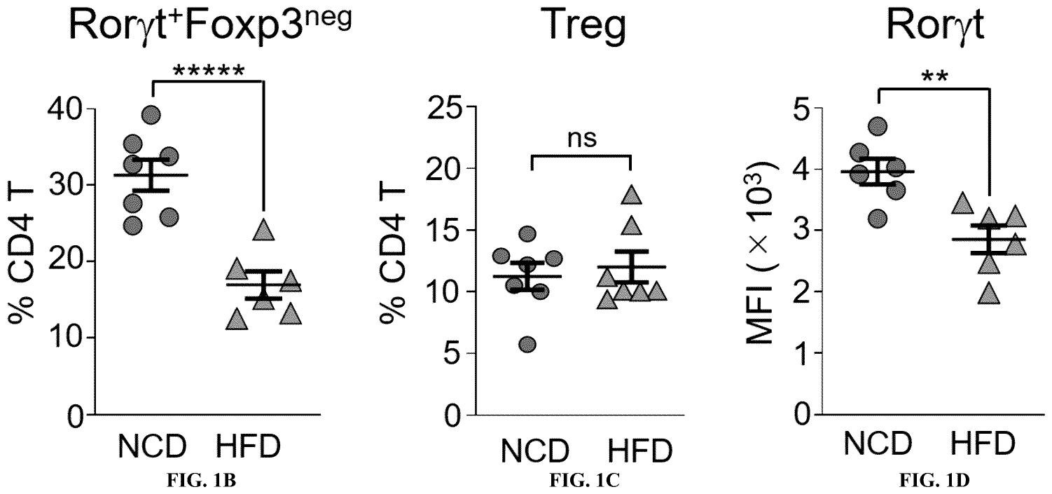

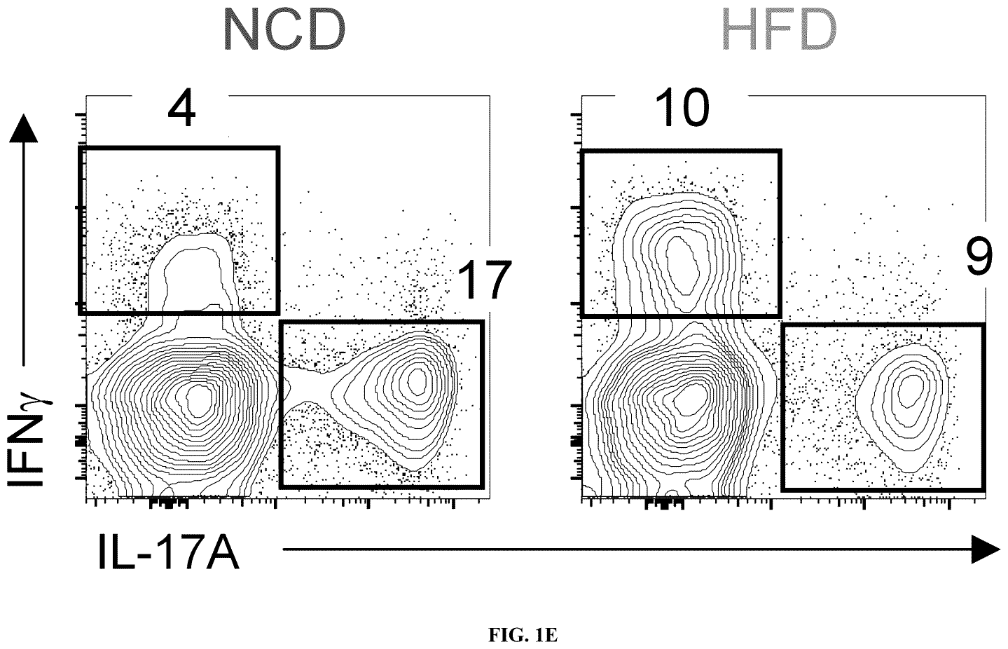

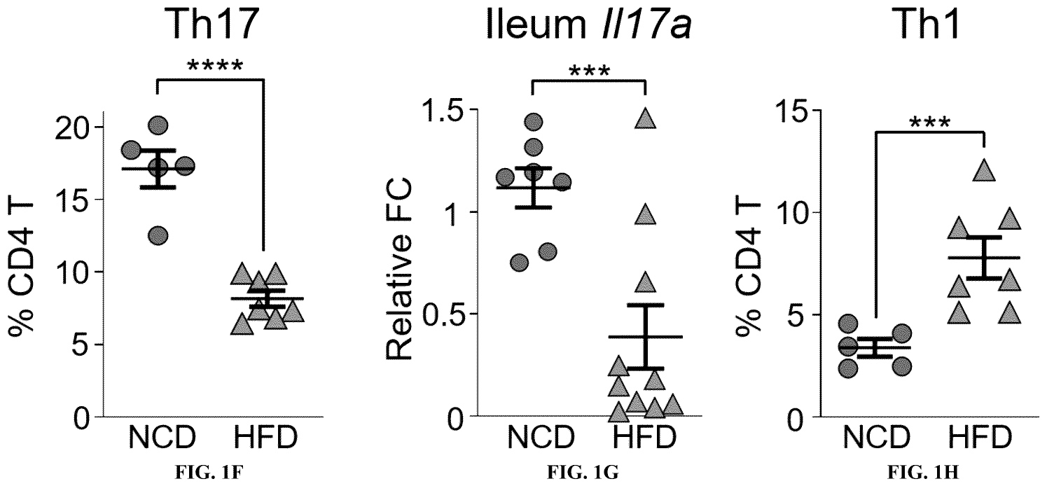

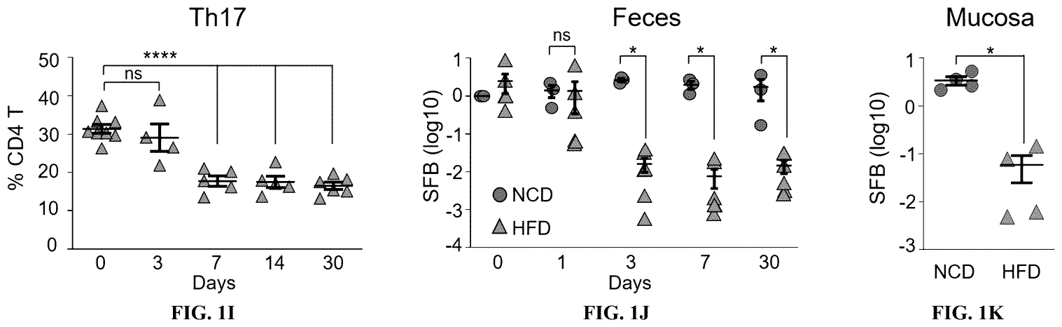

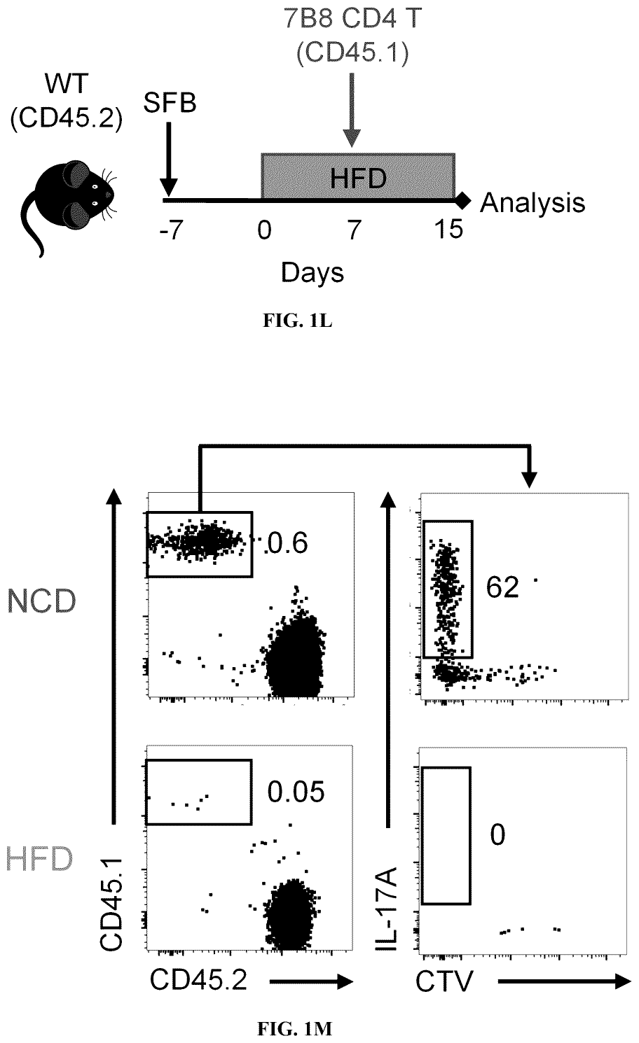

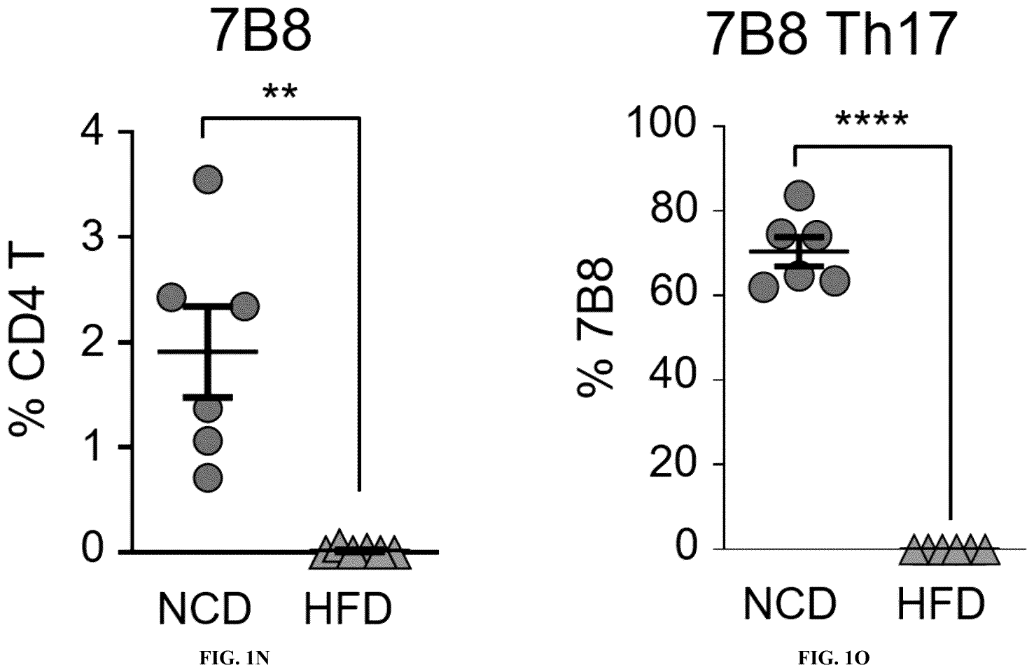

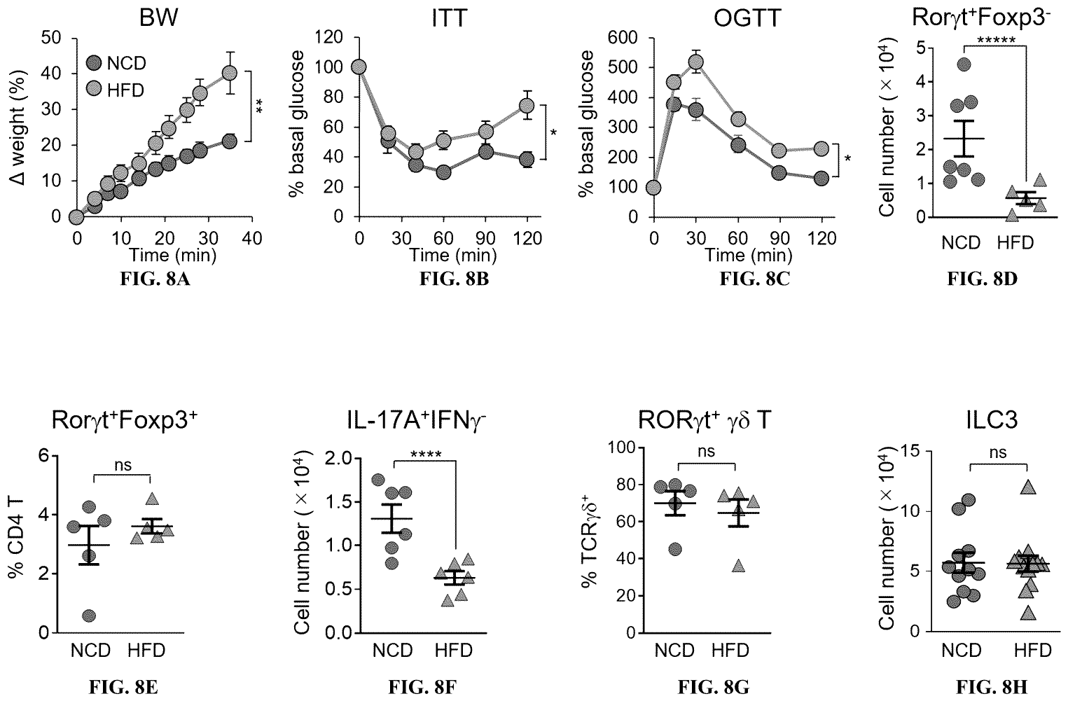

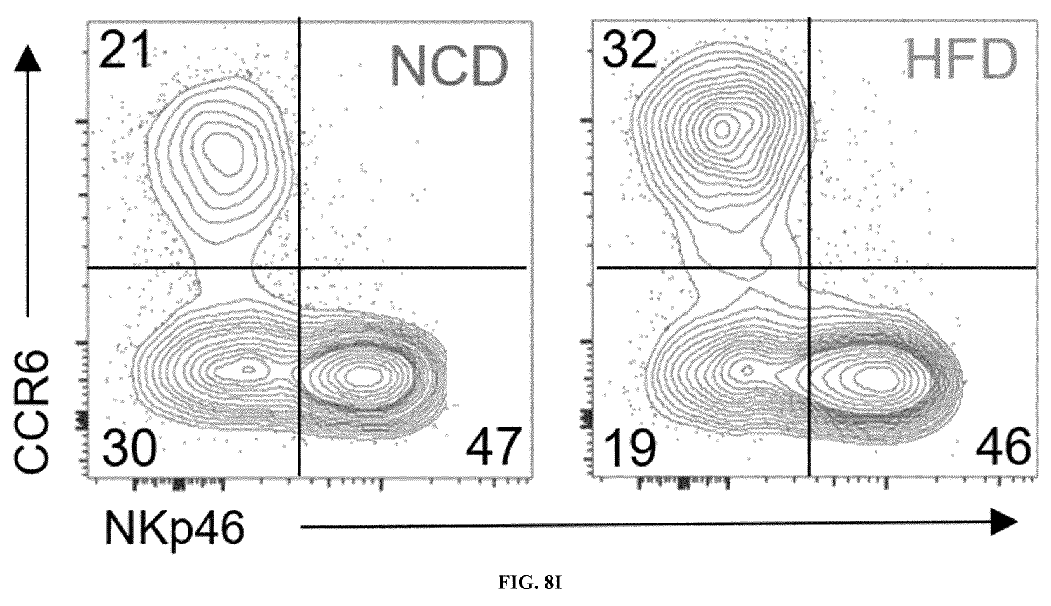

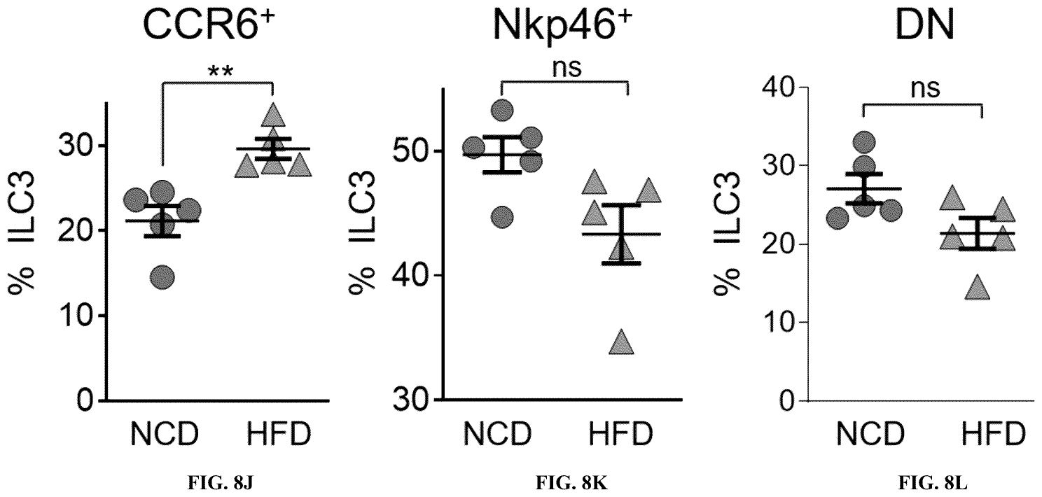

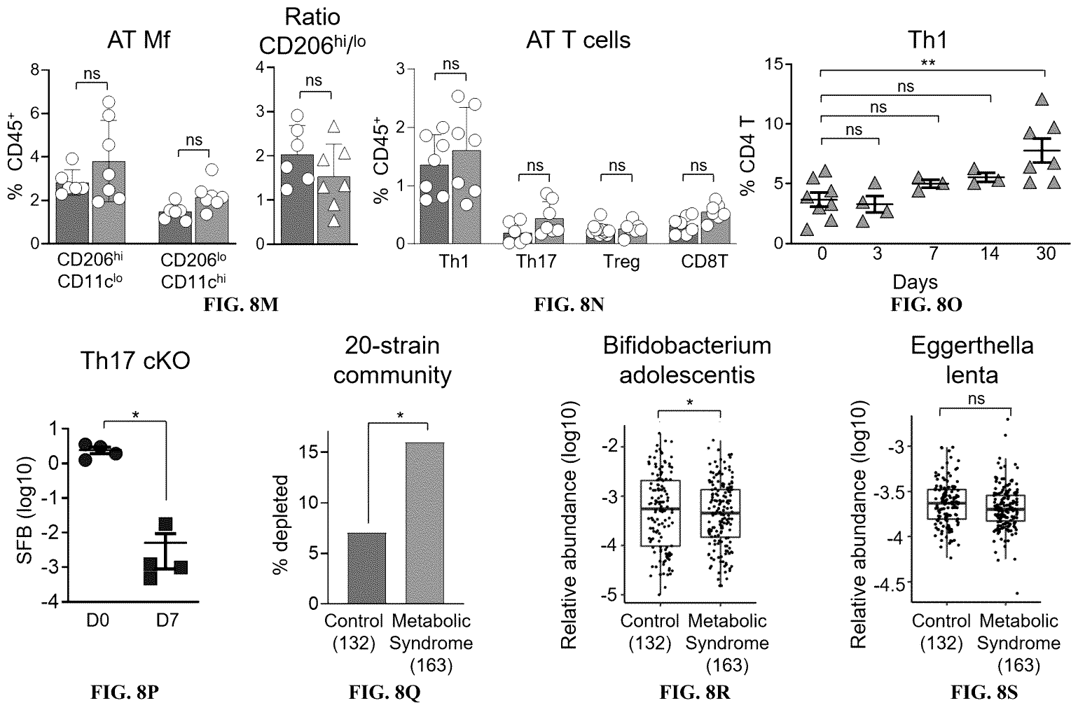

To identify initiating events in metabolic syndrome, the effects of HFD on intestinal immune homeostasis were examined at four weeks, prior to development of inflammatory changes in adipose tissue. Compared to normal chow diet (NCD), WT mice fed HFD developed metabolic syndrome characteristics, including weight gain, insulin resistance, and glucose intolerance (FIGS. 8A-8C). In the small intestinal lamina propria (SI LP) HFD led to significant decrease in the proportion and total numbers of RORγt⁺Foxp3^{neg} Th17 cells (FIGS. 1A, 1B, and 8D), but had no apparent effect on RORγt^{neg} or RORγt⁺ Foxp3⁺ Tregs (FIGS. 1C and 8E). Moreover, remaining RORγt⁺ Th17 cells had decreased expression of RORγt (FIG. 1D), suggesting general loss of Th17 cell functionality. Cytokine staining revealed corresponding decrease in percentage and total numbers of IL-17+Th17 cells (FIGS. 1E, F and 8F) and severely reduced tissue levels of Il17 transcripts in the terminal ileum (FIG. 1G) in HFD-fed animals. At the same time, HFD did not affect the levels of other RORγt or IL-17-expressing populations, such as RORγt⁺ γδ T cells or total ILC3 (FIGS. 8G and 8H). However, HFD feeding was associated with an increase in the proportion of SI LP Th1 cells (FIG. 1H), as well as a relative enrichment of CCR6+ILC3 (FIG. 81-8L), a subset that produces high levels of IL-22 (Klose and Artis, 2016).

As expected, at four weeks, major inflammatory immune cell subsets in visceral adipose tissue were not significantly changed (FIGS. 8M and 8N). Therefore, HFD leads to specific decrease in intestinal Th17 cell immunity. The loss of SI LP Th17 cells occurred by Day 7 following transition to HFD (FIG. 1I) and preceded the increase in inflammatory LP Th1 cells (FIG. 8O).

It has been shown that SI LP Th17 cells in SPF mice are induced by commensal microbiota, particularly SFB (Goto et al., 2014; Ivanov et al., 2009). Therefore, whether HFD affects SFB levels was investigated. Transition to HFD led to rapid loss of SFB from both feces and ileal mucosa (FIGS. 1J and 1K). Notably, SFB loss preceded the loss of Th17 cells (FIGS. 1I and 1J) and SFB loss still occurred in Th17 cell-deficient animals (FIG. 8P). Thus, the decrease in SI LP Th17 cells following transition to HFD is secondary to the loss of SFB. To confirm that HFD-induced SFB loss eliminates induction of SFB-specific Th17 cells, congenic SFB-specific 7B8 TCR Tg T cells (Yang et al., 2014) were adoptively transferred into SFB-positive mice fed NCD or HFD (FIG. 1L). 7B8 CD4 T cells expanded and differentiated into Th17 cells in NCD controls (FIGS. 1M-1O). In contrast, SFB-specific CD4 T cells did not expand or generate Th17 cells in HFD-fed animals (FIGS. 1M-1O). These results demonstrate that both SFB and SFB-derived T cell antigens are lost following transition to HFD. Collectively, these data suggest that HFD induces rapid loss of Th17 cell-inducing microbiota that leads to loss of homeostatic commensal Th17 cells prior to development of metabolic syndrome.

The abundance of previously reported human Th17 cell-inducing bacteria was also investigated in a published microbiota dataset from non-diabetic adults with or without increased body mass index and metabolic syndrome (Pedersen et al., 2016). A significantly higher proportion of adults with metabolic syndrome showed depletion of community of 20 human Th17-inducing bacteria (Atarashi et al., 2015) (FIG. 8Q). Metabolic syndrome adults also had decreased relative abundance of Bifidobacterium adolescentis (Tan et al., 2016) but not Eggerthella lenta (Alexander et al., 2022) (FIGS. 8R and 8S). Therefore, metabolic syndrome may also negatively affect Th17-inducing microbiota in humans.

### Example 2. Microbiota-Induced Th17 Cells Protect from Metabolic Syndrome

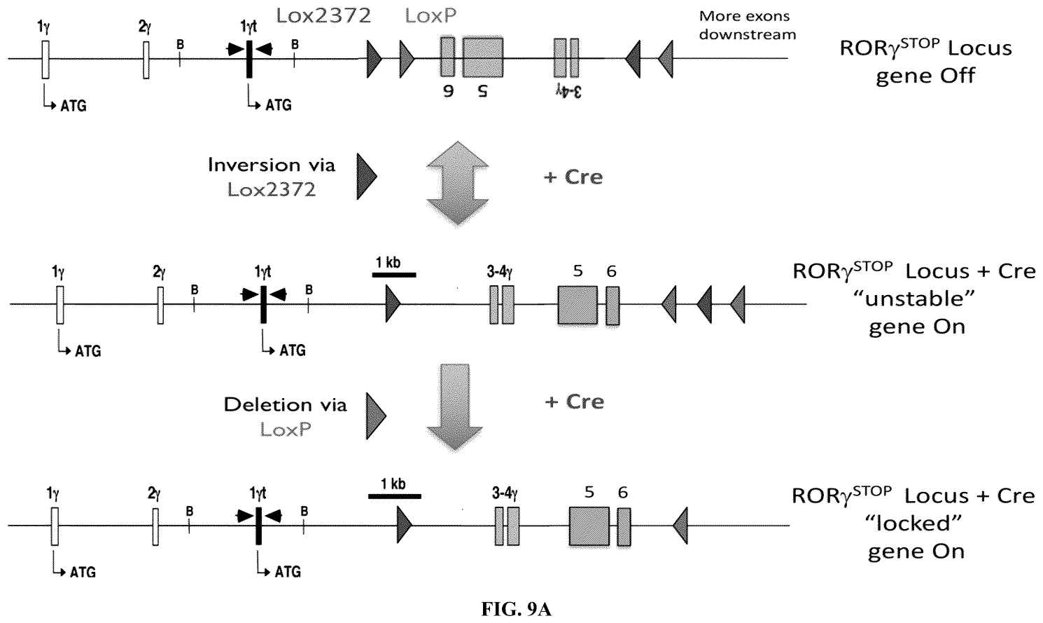

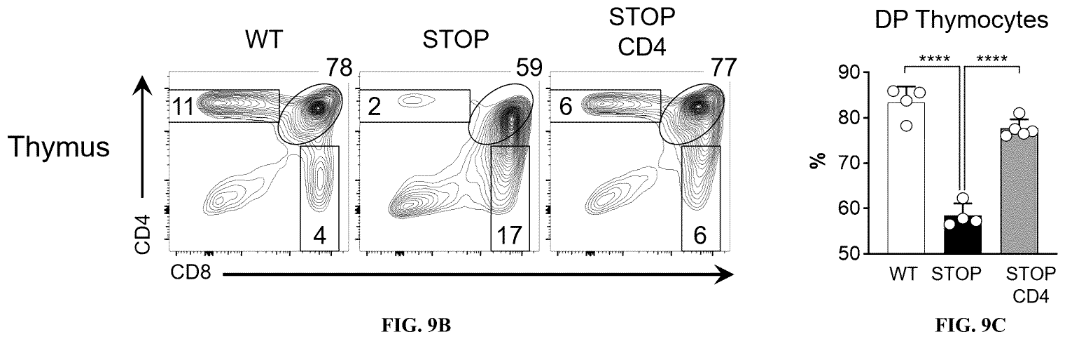

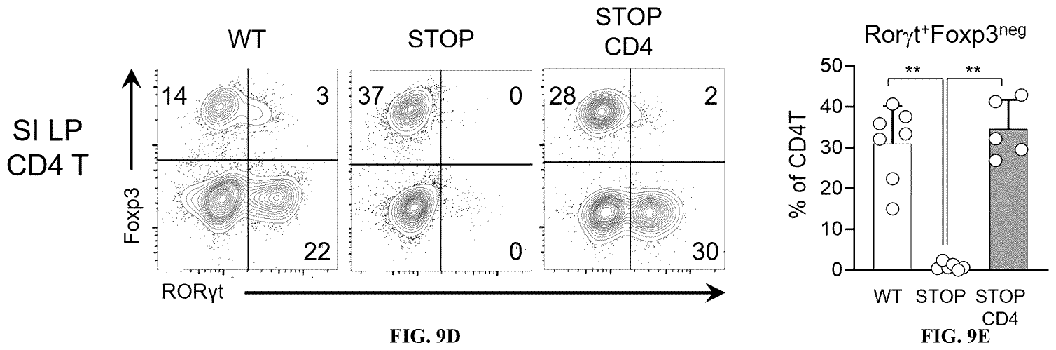

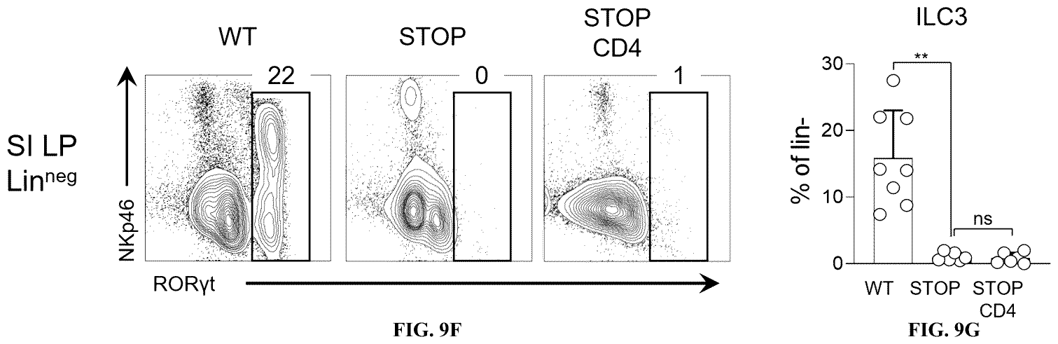

Both Th17 cells and ILC3 have been implicated in protection from metabolic syndrome (Garidou et al., 2015; Wang et al., 2014) and are regulated by SFB (Ivanov et al., 2009; Sano et al., 2015). Therefore, the differential role of Th17 cells and ILC3 was examined in metabolic syndrome. Traditionally, this has been difficult to ascertain, because all currently available ILC3-depletion models also have perturbed T cell development and/or Th17 differentiation (Klose and Artis, 2016; Tait Wojno and Artis, 2016; Vivier et al., 2018). A genetic model in which ILC3 development is selectively impaired while preserving the T cell compartment was generated (FIGS. 9A-9H). First, RORγ-STOP-flox (STOP) mice that lack both ILC3 and Th17 cells (FIG. 9A) was generated. These animals phenocopy RORγ-KO animals (FIGS. 9B-9G). They have perturbed T cell development in the thymus, and do not generate Th17 cells (including SI LP Th17 cells) or ILC3 (FIGS. 9B-9G). STOP mice were crossed to T cell-specific CD4-Cre animals to recover RORY expression in DP thymocytes (hence in all T cells). The resulting STOP/CD4-Cre (STOP/CD4) mice recover most αβ T cell development, recover SI LP Th17 cell differentiation, but maintain other immune deficiencies present in STOP mice, including the lack of ILC3 (FIGS. 9B-9G).

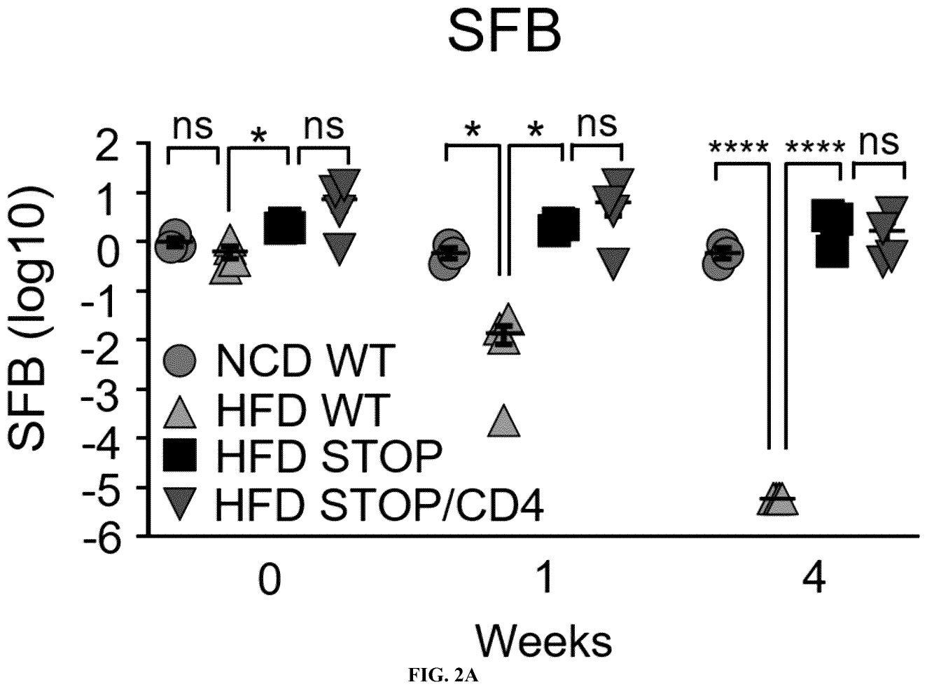

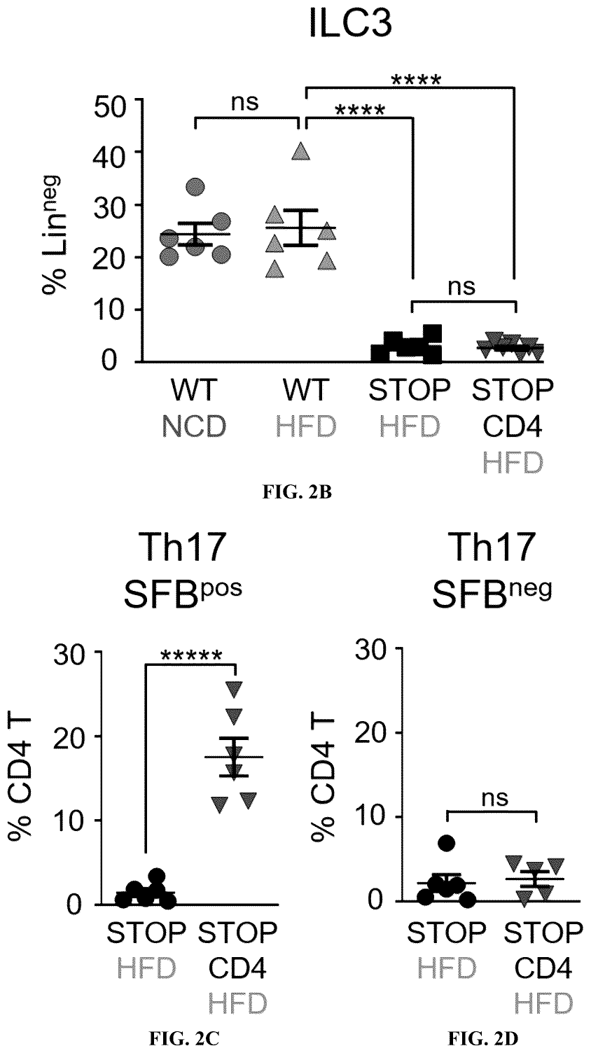

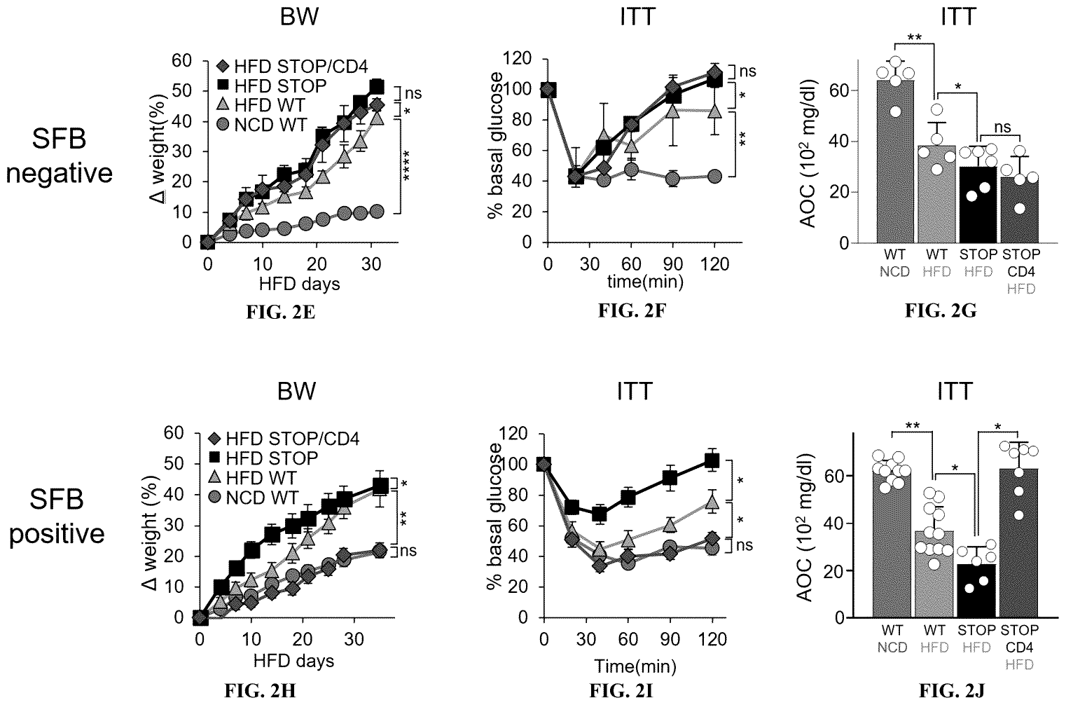

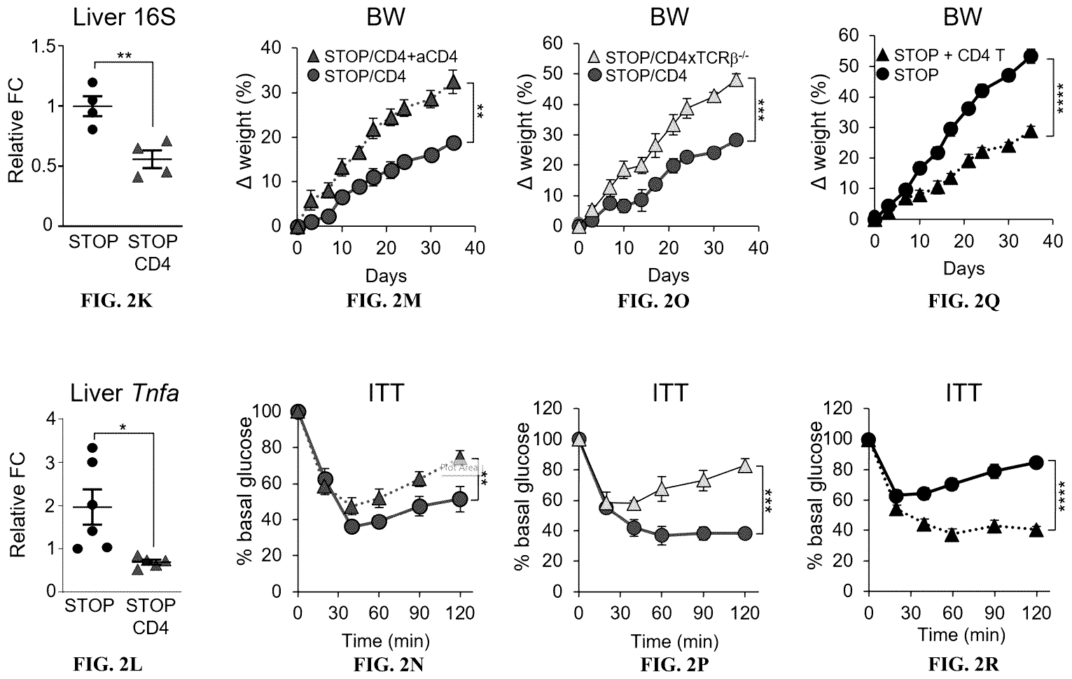

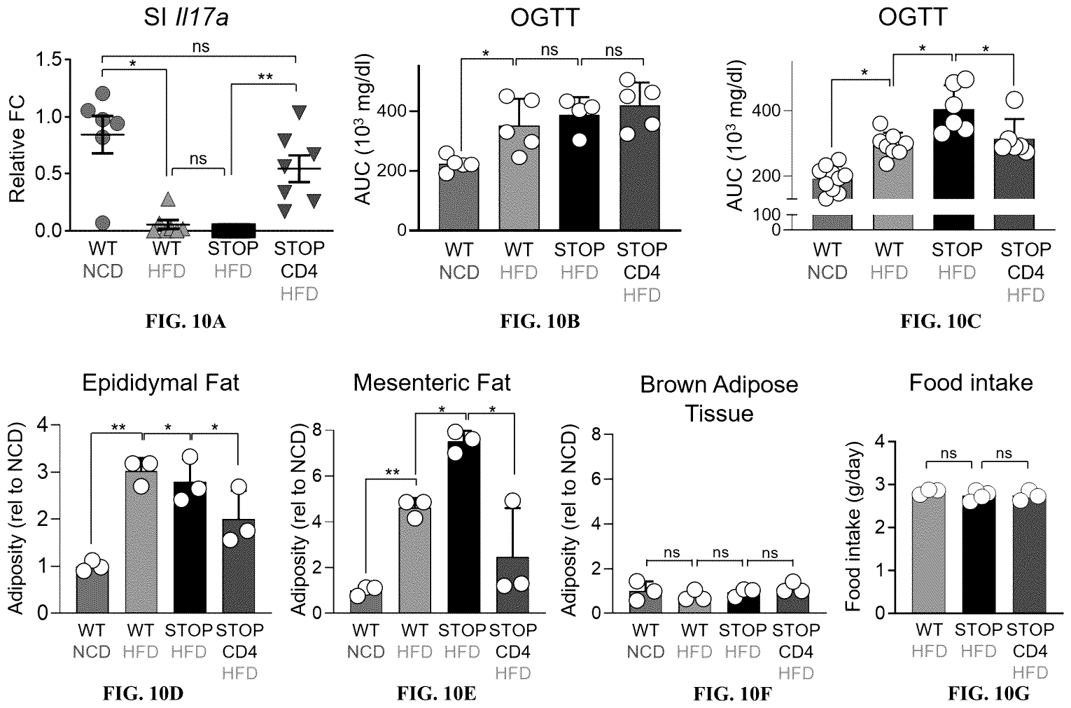

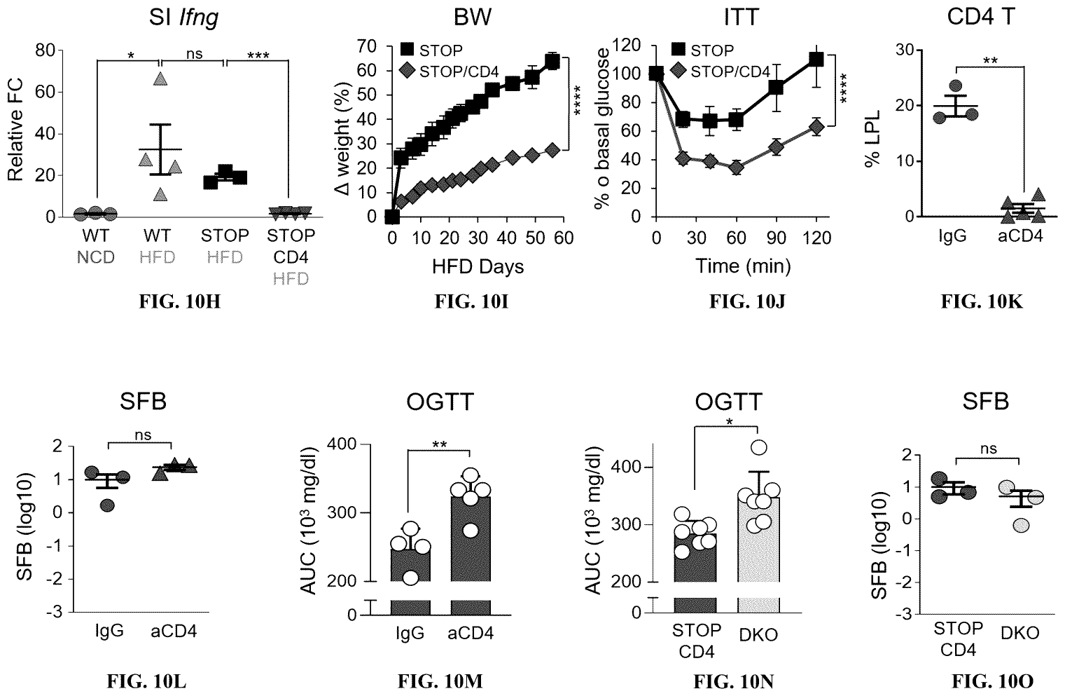

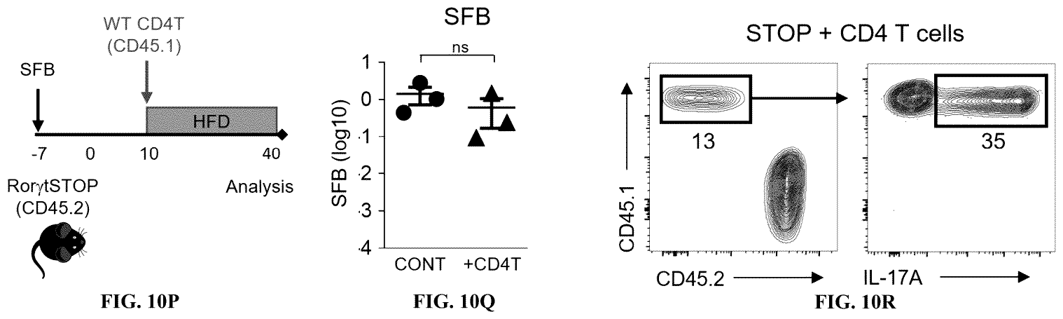

SFB-negative STOP, STOP/CD4, and WT littermate controls were colonized with SFB and fed HFD. After transition to HFD, WT animals quickly lost SFB as before. In contrast, HFD did not lead to loss of SFB in ILC3-deficient mice (STOP or STOP/CD4) (FIGS. 2A and 2B), suggesting that ILC3 are required for the HFD-mediated loss of SFB. Irrespective of SFB, HFD-fed STOP mice did not generate Th17 cells and had decreased levels of Il17a transcripts in the terminal ilcum (FIGS. 2C, 2D, and 10A). In contrast, STOP/CD4 mice colonized with SFB maintained high levels of SI LP Th17 cells even under HFD (FIGS. 2C and 10A). As expected, SFB-negative STOP/CD4 mice lacked SI LP Th17 cells (FIG. 2D).

Next, the development of DIO and metabolic syndrome in ILC3/Th17-deficient STOP mice and ILC3-deficient/Th17-sufficient STOP/CD4 mice were compared. In the absence of SFB-induced Th17 cells (FIG. 2D), both strains of ILC3-deficient mice demonstrated weight gain (FIG. 2E) and metabolic syndrome phenotypes, i.e. increased insulin resistance and glucose intolerance (FIGS. 2F, 2G, and 10B). Moreover, weight gain and metabolic syndrome phenotypes in ILC3-deficient STOP and SFB-negative STOP/CD4 mice were slightly, but significantly, increased compared to WT controls (FIGS. 2E-J, 10B, and 10C). In the presence of SFB Th17 cells (FIG. 2C), STOP/CD4 mice resembled NCD-fed WT controls and were protected from DIO, including weight gain (FIG. 2H) and increased adiposity (FIGS. 10D and 10E), as well as pre-diabetic phenotypes associated with metabolic syndrome (FIGS. 2I, 2J, and 10C). Protection was not mediated by changes in brown fat adiposity or food intake (FIGS. 10F and 10G). In addition to maintaining SI LP Th17 cells (FIG. 2C), HFD-fed SFB-positive STOP/CD4 mice had significantly decreased levels of transcripts for the Th1 cytokine IFNγ in the SI compared to HFD-fed WT or STOP mice (FIG. 10H). They also demonstrated decreased liver pathology, including decreased bacterial translocation and expression of Infa transcripts (FIGS. 2K and 2L). The protection from metabolic syndrome in SFB-positive STOP/CD4 mice was also evident at eight weeks (FIGS. 10I and 10J). Therefore, protection from DIO and metabolic syndrome in STOP/CD4 mice correlates with the presence of SFB-induced Th17 cells.

To confirm that protection is mediated by CD4 T cells, CD4 T cells were depleted in SFB/Th17-positive STOP/CD4 mice using anti-CD4 antibody (FIG. 10K) and administered HFD. Depletion of CD4 T cells did not affect SFB levels in HFD-fed STOP/CD4 mice (FIG. 10L). However, protection from DIO and metabolic syndrome was lost in CD4 T cell-depleted STOP/CD4 mice (FIGS. 2M, 2N, and 10M). STOP/CD4 mice were also crossed to TCRβ-KO animals to genetically delete αβ T cells. TCRβKO-STOP/CD4 animals became susceptible to DIO and metabolic syndrome (FIGS. 2O, 2P, and 10N), despite maintenance of SFB (FIG. 10O). Together, these experiments demonstrate that intestinal or commensal Th17 cells are required for microbiota-mediated protection against DIO and metabolic syndrome. Such Th17 cells could also be generated in vitro with the characteristics of natural intestinal or commensal TH17 cells.

To investigate whether Th17 cells are sufficient to bestow protection, WT CD4 T cells were transferred into SFB-colonized metabolic syndrome-susceptible STOP mice (FIG. 10P).

Transfer of CD4 T cells did not affect SFB levels (FIG. 10Q). Transferred WT CD4 T cells differentiated into Th17 cells locally in the SI LP (FIG. 10R; Goto et al., 2014; Sano et al., 2015)). STOP mice adoptively transferred with CD4 T cells were significantly protected from DIO and metabolic syndrome compared to untreated animals (FIGS. 2Q and 2R). The foregoing studies suggest that gut microbiota can mediate protection from metabolic syndrome through induction of intestinal Th17 cells. Microbiota-induced Th17 cells appear to be both necessary and sufficient to provide protection and prevent or suppress development of obesity and pre-diabetic phenotypes.

### Example 3. Probiotic Th17 Cell-Inducing Bacteria Ameliorate Metabolic Syndrome

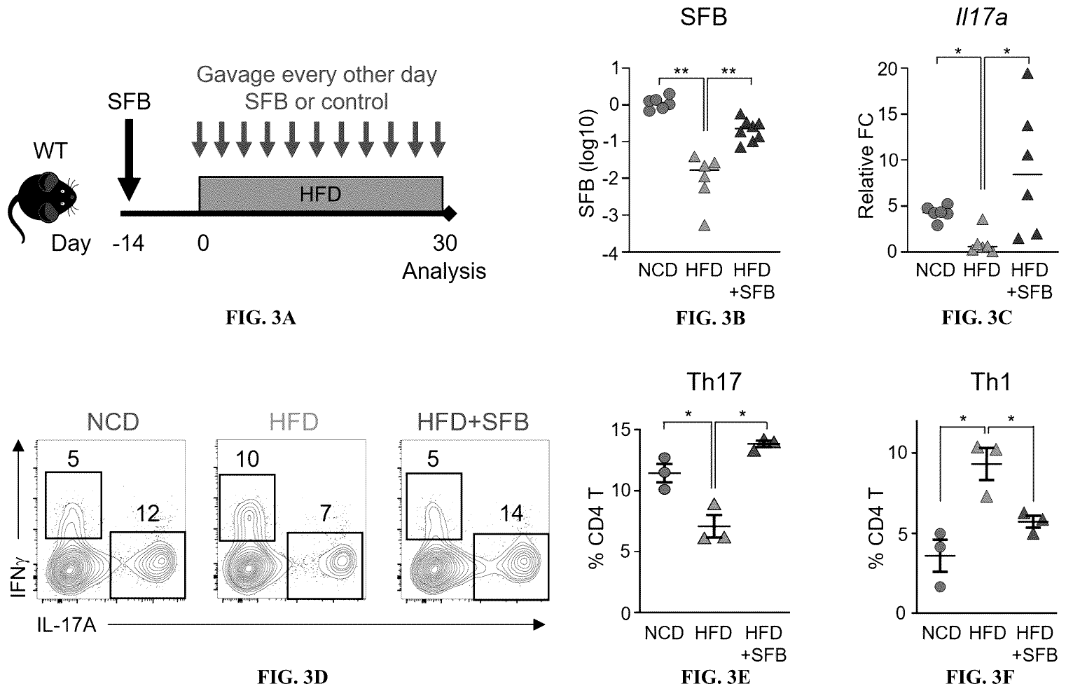

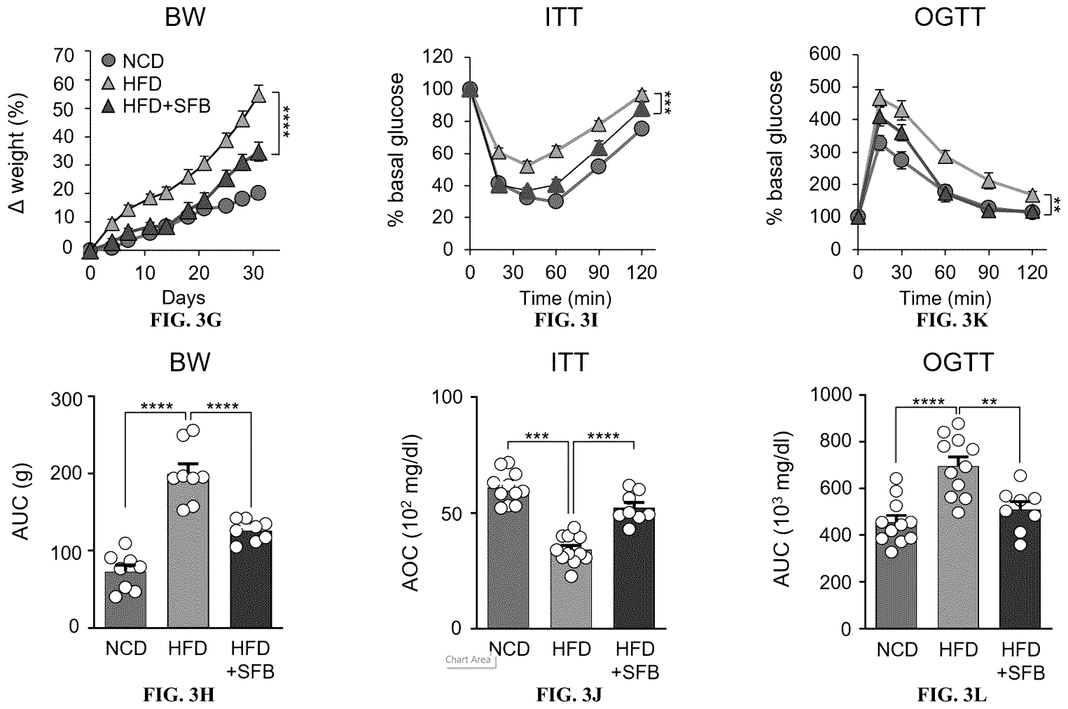

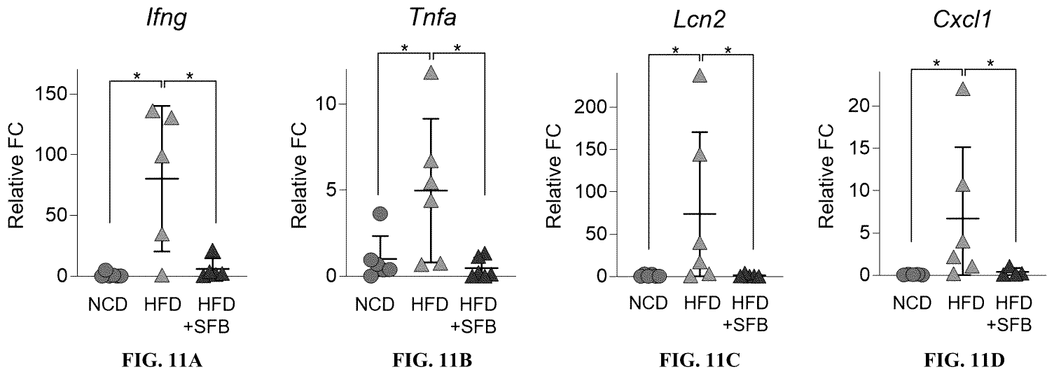

The results herein demonstrate that one of the pathogenic effects of HFD is depletion of homeostatic commensal intestinal Th17 cells through elimination of Th17 cell-inducing microbiota. Recovery of intestinal Th17 cells by maintaining Th17 cell-inducing microbiota under HFD may improve DIO and metabolic disease. Therefore, SFB-positive HFD-fed WT mice with SFB or control bacteria were treated by oral gavage every other day for four weeks (FIG. 3A). HFD-fed animals gavaged with control bacteria lost SFB (FIG. 3B), which led to decrease of intestinal Il17a transcripts (FIG. 3C), due to decrease of SI LP Th17 cells (FIGS. 3D and 3E). The animals also developed obesity and metabolic disease (FIGS. 3G-3L). SFB administration led to partial, but significant, recovery of SFB levels in fecal contents (FIG. 3B). Importantly, SFB-treated animals had significant recovery of SI LP Th17 cells (FIGS. 3D and 3E) and IL-17 expression in terminal ileum (FIG. 3C). Compared to controls, SFB-treated animals had significantly reduced weight gain under HFD (FIGS. 3G and 3H) and were protected from development of pre-diabetic phenotypes, including insulin resistance (FIGS. 31 and 3J) and glucose intolerance (FIGS. 3K and 3L). SFB-treated animals also showed amelioration of HFD-induced intestinal inflammation, including decrease in inflammatory Th1 cells (FIG. 3F), transcripts for inflammatory T cell cytokines, e.g. IFN-γ and TNF-α (FIGS. 11A and 11B), and transcripts for markers of tissue inflammation (FIGS. 11C and 11D). Thus, that a probiotic regimen of Th17 cell-inducing microbiota can significantly ameliorate DIO and metabolic syndrome by recalibrating intestinal T cell homeostasis.

### EXAMPLE 4. Dietary Sugar Promotes Metabolic Syndrome Through Elimination of Commensal Th17 Cells

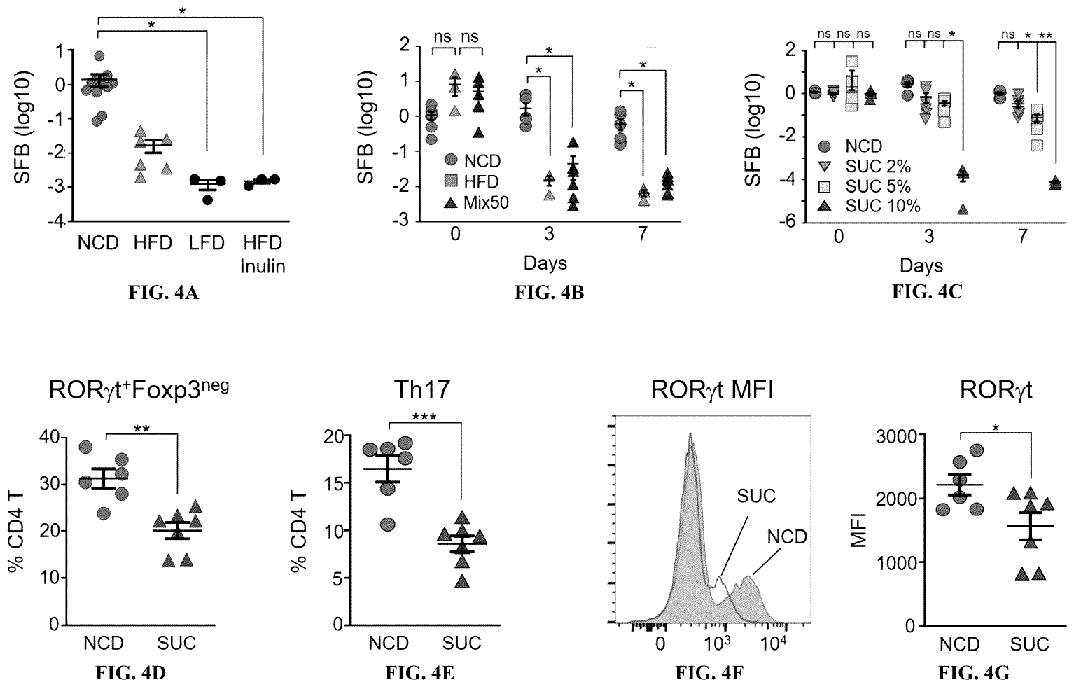

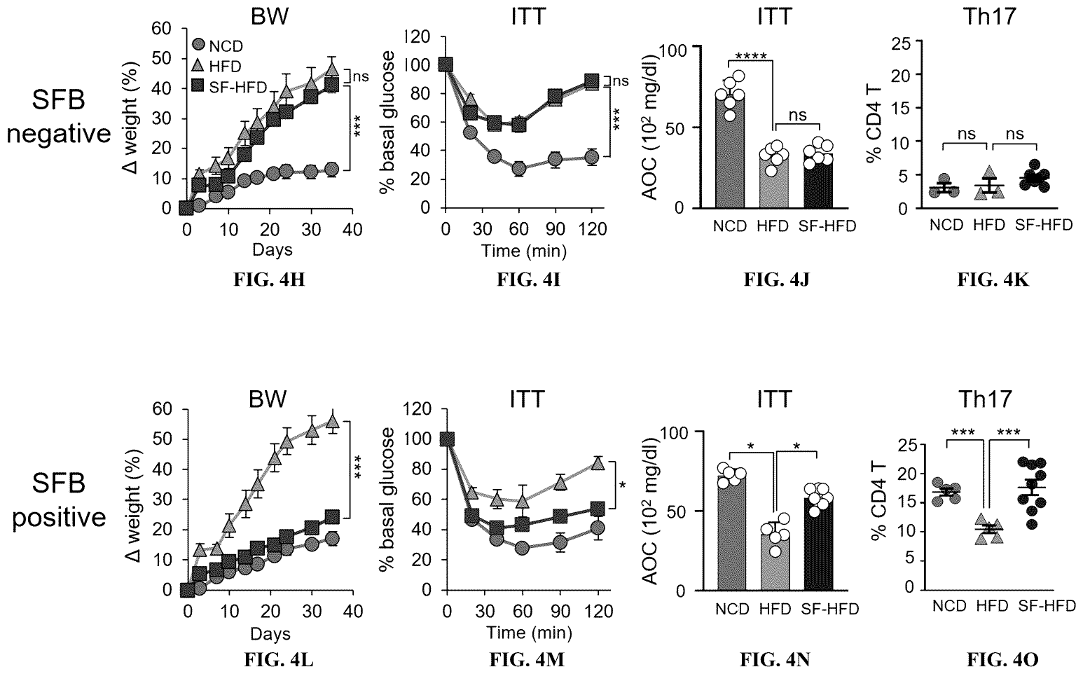

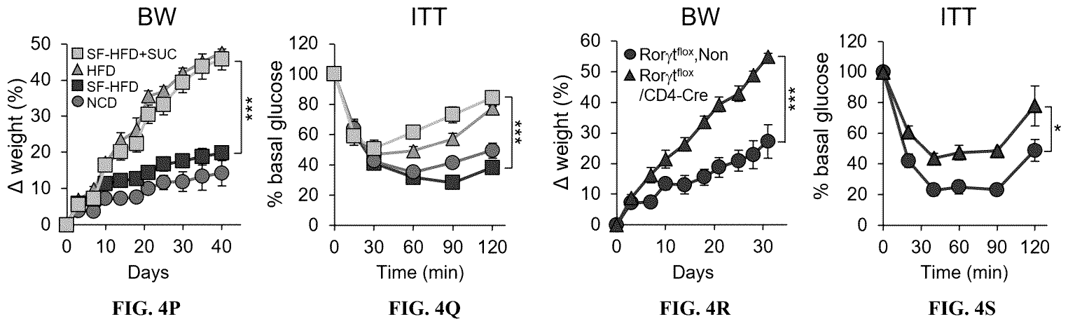

To investigate the nature of the dietary components that lead to loss of protective commensal intestinal Th17 cells, loss of SFB as a readout was used to identify dietary ingredients that deplete intestinal Th17 cells. Two of the better characterized deleterious nutritional components of HFD are excess fat and low dietary fiber. However, neither removal of excess fat from our HFD formulation by using control purified low-fat diet (LFD), nor the addition of dietary fiber in the form of inulin improved SFB maintenance (FIG. 4A). In contrast to less well-defined grain-based normal chow, HFD is a purified diet that contains defined ingredients. Therefore, numerous nutritional components differ between the two formulations.

To answer whether the loss of SFB is due to presence of an inhibitory activity or lack of a nutritional component in HFD, compared to NCD, mice were provided with both diets simultaneously. If HFD contains an excess of an inhibitory component, then it should still inhibit SFB even in the presence of NCD. Alternatively, a missing nutritional component will be recovered by complementation with NCD. WT mice were colonized with SFB and then fed NCD, HFD, or 50:50 Mix of the two diets (FIG. 4B). The addition of NCD nutritional components as a 50:50 NCD:HFD mix, did not prevent SFB decrease (FIG. 4B). This suggested that HFD contains an “inhibitory” component, prompting a focus on the ingredients enriched in the HFD formulation.

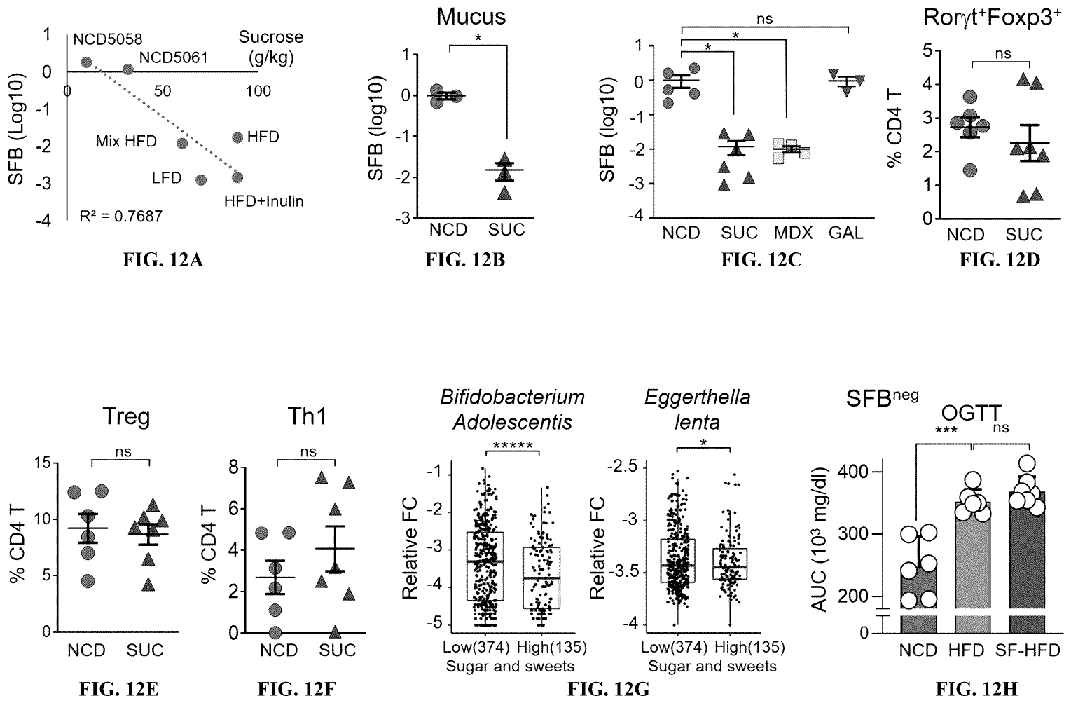

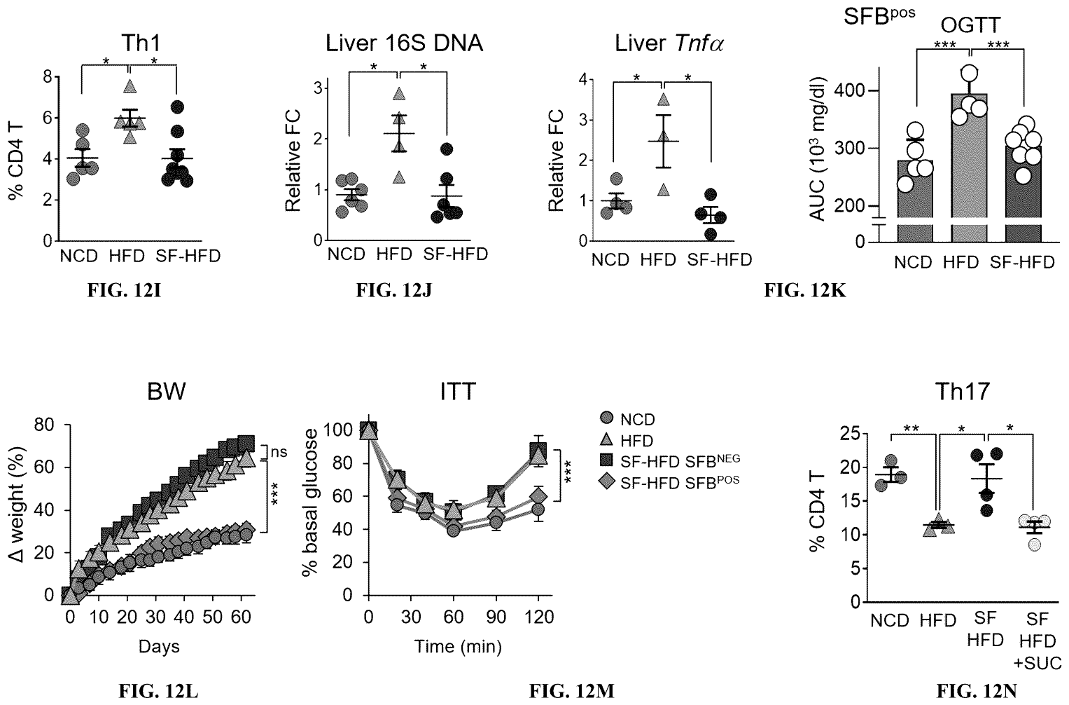

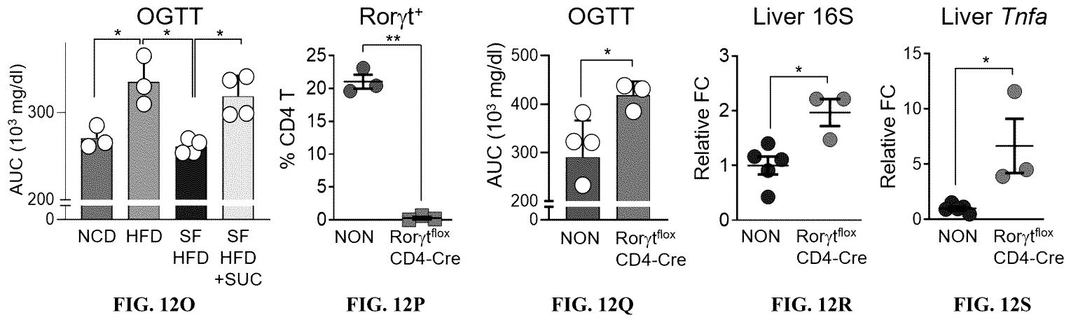

In addition to dietary fat, another ingredient highly represented in HFD is dietary sugar. While NCD formulations contain 3-6% sugar, HFD formulations contains 25% dietary sugars, including 10% sucrose and 15% maltodextrin. Sucrose and maltodextrin (a common ingredient in packaged foods, including candies and soft drinks) are thought to increase risk of metabolic syndrome, although the mechanisms remain controversial (Bravo et al., 2013; Johnson et al., 2013; Macdonald, 2016; Malik et al., 2010). Sugar levels in diet formulations inversely correlated with diets' effects on SFB levels (FIG. 12A). Sucrose, provided ad libitum into the drinking water of NCD-fed WT animals, eliminated SFB in a dose-dependent manner (FIG. 4C). 10% w/v sucrose or maltodextrin decreased SFB levels in feces and ileal mucosa of NCD-fed mice with similar kinetics to HFD-fed animals (FIGS. 4C, 12B, and 12C). In contrast, 10% galactose did not significantly affect SFB levels (FIG. 12C). Next, the effects of sucrose on intestinal Th17 cells were examined.

Addition of sucrose to the water of NCD-fed animals decreased both RORγt⁺ and IL-17+Th17 cells in SI LP (FIGS. 4D and 4E), similarly to HFD (compare to FIGS. 1A-10). As observed with HFD, the remaining Th17 cells in sucrose-fed animals had decreased levels of RORγ, suggesting perturbed functionality (FIGS. 4F and 4G). Other intestinal CD4 T cell subsets were not significantly affected by sucrose (FIGS. 12D-12F). Examination of a published human personalized diet-microbiome dataset (Johnson et al., 2019) revealed that increased sugar consumption was associated with decrease in the relative abundance of Th17 cell-inducing microbes in human volunteers (FIG. 12G).

To investigate the role of dietary sugar on Th17 cell-mediated protection, sugar-free HFD (SF-HFD) was generated by replacing sucrose and maltodextrin in HFD with starch. SF-HFD induced obesity and metabolic syndrome similarly to control HFD in SFB-negative animals (FIGS. 4H-4J and 12H). In contrast, SFB-colonized SF-HFD-fed mice were protected from weight gain (FIG. 4L), insulin resistance (FIGS. 4M and 4N), and glucose intolerance (FIG. 12H). In contrast to HFD-fed mice, SFB-positive SF-HFD-fed animals maintained high levels of protective intestinal Th17 cells (FIGS. 4K and 4O). SFB-positive SF-HFD-fed animals also lacked evidence of intestinal and systemic inflammation and had low levels of inflammatory Th1 cells in SI LP (FIG. 12I) and decreased levels of bacterial DNA and Infa transcripts in liver (FIGS. 12J and 12K). Protection was also present at nine weeks (FIGS. 12L and 12M).

To confirm that the positive effects of SF-HFD are due to absence of sugar and to control for other potential diet effects, a separate set of experiments was performed in which some SF-HFD animals received 10% sucrose in the drinking water. As before, SFB-colonized SF-HFD-fed animals maintained intestinal Th17 cells and were protected from DIO and metabolic syndrome (FIGS. 4P and 4Q). The protection afforded SF-HFD-fed mice, however, was entirely lost when sugar was added to their drinking water. The animals lost intestinal Th17 cells and were as susceptible as HFD-fed animals to obesity and metabolic syndrome (FIGS. 4P, 4Q, 12N, and 12O).

To confirm that the protective effects of SF-HFD are mediated by Th17 cells we examined RORγt-flox/CD4-Cre animals, that specifically lack Th17 cell differentiation (Choi et al., 2016). RORγt-flox/CD4-Cre and control littermates were colonized with SFB and fed SF-HFD. As expected, RORγt-flox/CD4-Cre animals lacked intestinal Th17 cells (FIG. 12P). At the same time protection from DIO and metabolic syndrome was lost in these animals compared to control WT littermates (FIGS. 4R and 4S). Compared to WT littermates, Th17 cell-deficient mice on SF-HFD showed increased weight gain (FIG. 4R), insulin resistance (FIG. 4S), glucose intolerance (FIG. 12Q), and increased bacterial translocation and inflammatory markers in liver (FIGS. 12R and 12S).

Collectively, the foregoing data suggest that dietary sugar counteracts the protective effects of intestinal commensal Th17 cells in the context of DIO and metabolic syndrome by depleting these cells. They also show that elimination of dietary sugar is not sufficient to provide therapeutic benefit; protection also requires presence of intestinal or commensal Th17 cells, whether produced naturally or in vitro. Therefore, dietary interventions may need to be combined with immune therapies to achieve desired effects.

### Example 5. Dietary Sugar Displaces Th17 Microbiota by Increasing a Member of Erysipelotrichaceae

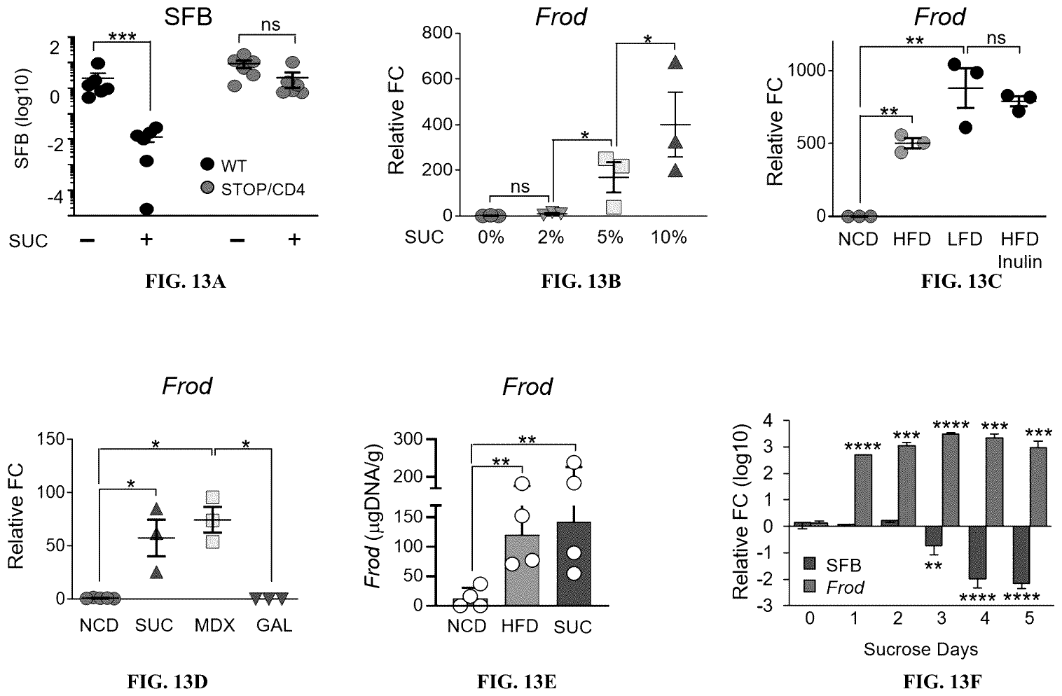

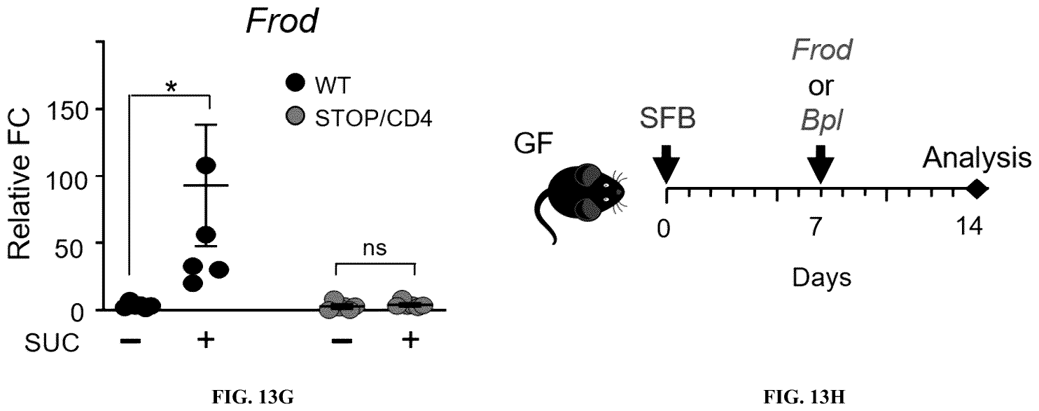

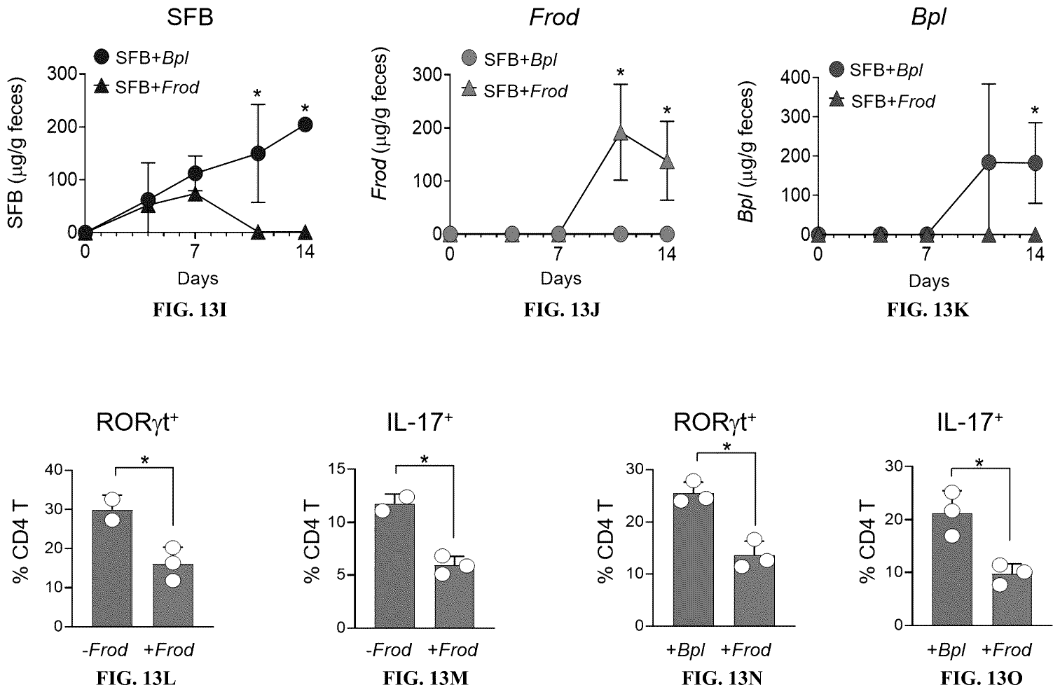

The mechanism by which dietary sugar displaces SFB and metabolic syndrome-protective Th17 cells was investigated. Similar to effects with HFD, sugar did not decrease SFB in ILC3-deficient mice (FIG. 13A), suggesting that sugar does not directly affect these Th17-inducing bacteria. To account for host effects, we treated SFB-monocolonized WT animals with 10% sucrose in the drinking water.

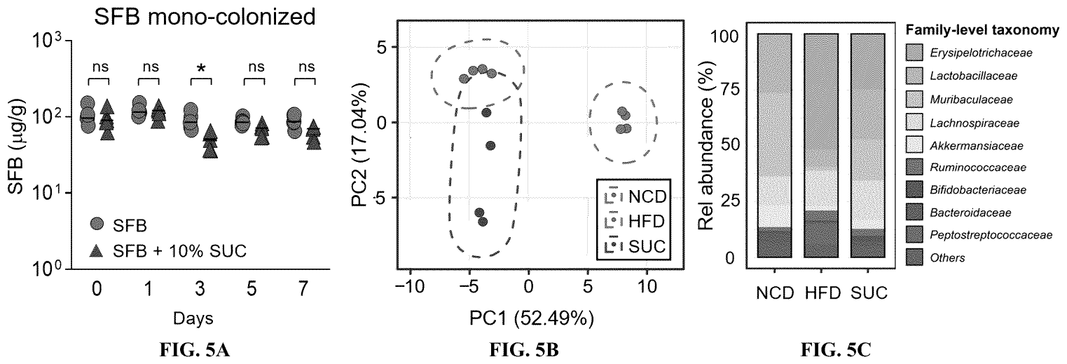

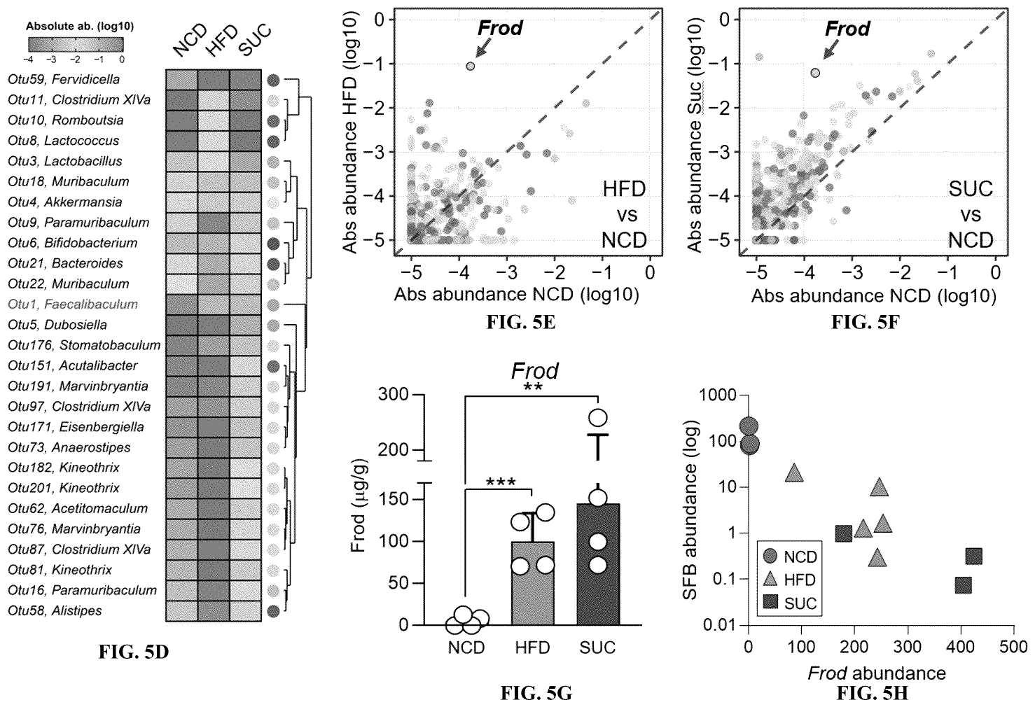

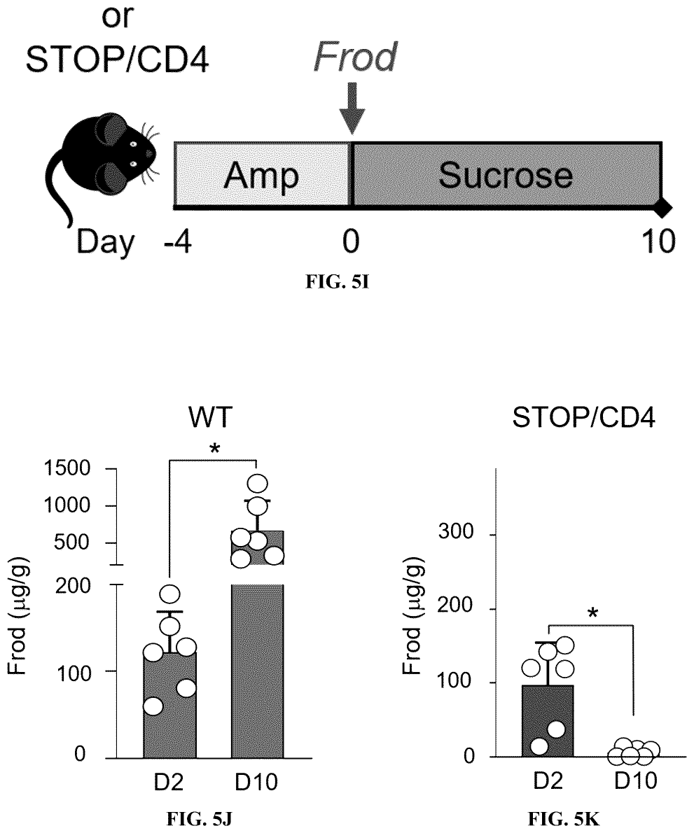

In contrast to SPF mice (FIG. 4C), dietary sugar did not affect SFB levels in monocolonized mice (FIG. 5A). Therefore, displacement of SFB by sugar requires the presence of commensal microbes. To identify commensal species that mediate the effects of sugar we compared microbiota composition of animals fed NCD, HFD, or NCD+10% sucrose in the drinking water (FIGS. 5B-5F). HFD-fed and sugar-treated animals had distinct microbiota composition from NCD-fed animals, but also significantly differed from each other (FIG. 5B). This allowed us to narrow down microbiota differences between Th17-depleting (HFD and sugar) and Th17-supporting (NCD) diets.

At the family level, Erysipelotrichaceae, Ruminococcaceae and Lachnospiraceae were upregulated in both Th17-depleting diets (FIG. 5C). Erysipelotrichaceae was by far the highest and most significantly enriched family in both HFD and sugar over NCD (FIG. 5C). Erysipelotrichaceae expansion has been reported in metabolic disorders, including DIO in mice (Turnbaugh et al., 2008), as well as in obese humans (Zhang et al., 2009). The Erysipelotrichaceae expansion in our dataset contained several operational taxonomic units (OTU). However, one particular OTU, identified as Frod, was consistently overrepresented in both HFD and sugar-treated animals (FIGS. 5D-5F). Expansion of Frod in HFD and sugar-treated mice was confirmed by quantitative PCR (FIG. 5G).

Comparison of SFB and Frod levels in individual animals identified strong inverse correlation between the two microbiota members (FIG. 5H). Sugar-mediated Frod expansion was dose-dependent (FIG. 13B) and was also present in other dietary treatments that eliminated SFB (FIG. 13C), but not in dietary treatments that maintained SFB (FIG. 13D). HFD-mediated expansion of Frod did not require SFB (FIG. 13E). Moreover, it preceded the loss of SFB in SFB-positive animals (FIG. 13F).

Therefore, Frod expansion may be responsible for the loss of SFB in SPF mice. In agreement with this hypothesis neither sugar, nor HFD, increased Frod in ILC3-deficient mice, which maintain SFB (FIG. 13G). To investigate whether this is due to inability of Frod to colonize or expand in ILC3-deficient mice we compared colonization kinetics following dietary intervention.

For this, endogenous Frod was eliminated by pre-treating WT and ILC3-deficient STOP/CD4 mice with ampicillin. Next, exogenous Frod was introduced by oral gavage and followed colonization kinetics. To examine Frod expansion, animals were also given 10% sucrose in the drinking water (FIG. 5I). Shortly after Frod gavage (Day 2), both WT and STOP/CD4 mice showed similarly high levels of Frod in feces (FIGS. 5J and 5K) suggesting that Frod is capable of colonizing ILC3-deficient animals. Sugar in the drinking water led to a robust expansion of Frod in WT mice by Day 10 (FIG. 5J). In contrast, Frod was almost undetectable at Day 10 in sugar-treated STOP/CD4 mice (FIG. 5K). The foregoing results demonstrate that sugar-mediated expansion of Frod requires ILC3.

The above results strongly suggest that sugar or HFD-mediated expansion of Frod is responsible for the loss of SFB. Therefore, gnotobiotic animals was used to directly test whether Frod is sufficient to outcompete or displace SFB.

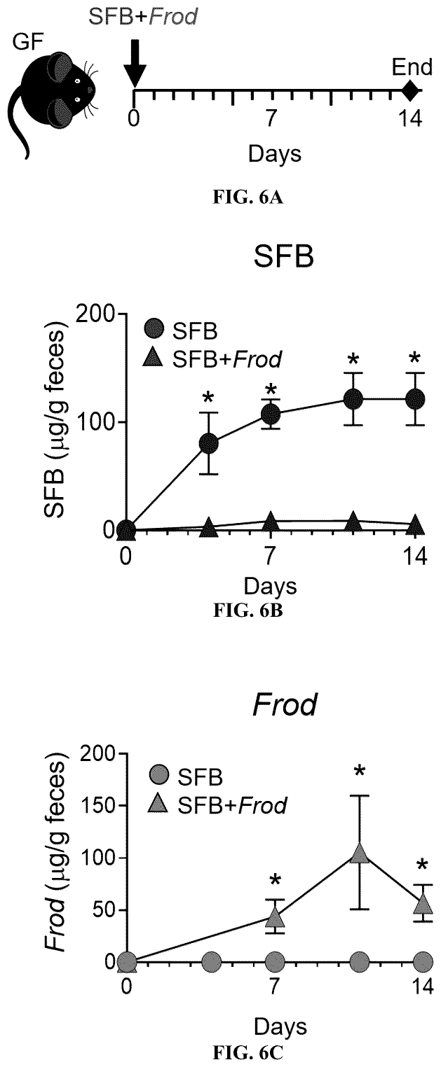

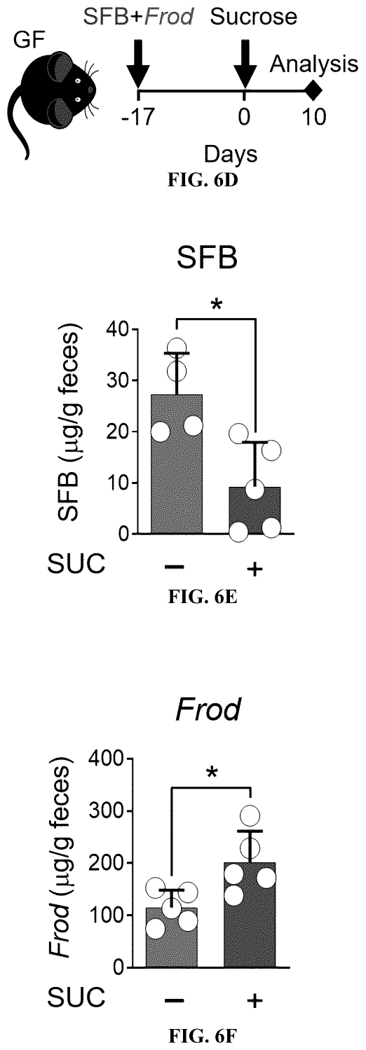

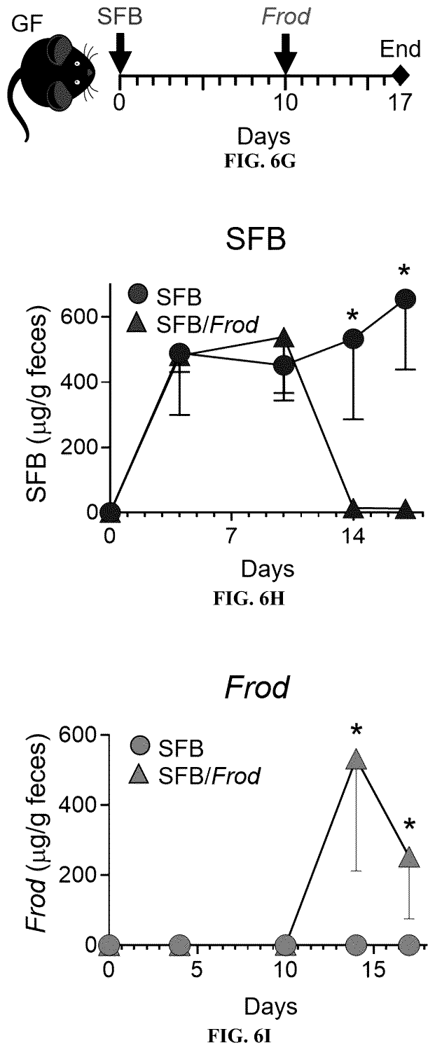

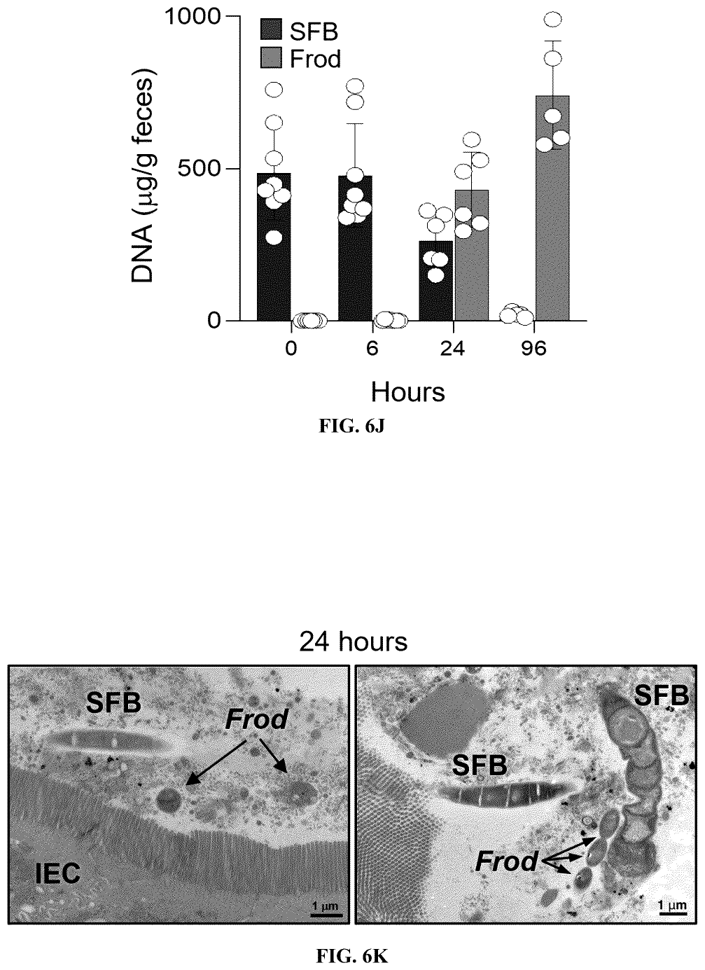

First, germ-free (GF) mice were gavaged with both with SFB and Frod at the same time to enable colonization (FIG. 6A). SFB colonized GF animals to high levels when provided alone (FIG. 6B). In contrast, SFB levels were 100-fold lower when SFB and Frod were gavaged together (FIGS. 6B and 6C). Addition of sugar to the drinking water of SFB/Frod dually colonized animals led to expansion of Frod and even further decrease in SFB levels (FIGS. 6D-6F). We also asked whether Frod can displace pre-existing SFB. GF animals were first colonized with SFB and later colonized with Frod or control bacteria (FIGS. 6G and 13H). All animals were similarly colonized with SFB prior to the introduction of Frod (FIGS. 6H and 13I). However, SFB quickly disappeared after introduction of Frod (FIGS. 6H and 61), but not after introduction of control bacteria (FIG. 13I-13K).

In accordance with the loss of SFB, Frod colonization was also accompanied by decrease of SFB-induced intestinal Th17 cells (FIGS. 13L-13O). 24 hours after Frod gavage, SFB and Frod were present together in gnotobiotic animals and occupied the same geographical niche in the mucus of terminal ileum close to epithelial cells and also in close proximity to each other (FIGS. 6J and 6K). These findings suggest that dietary sugar expands members of the Erysipelotrichaceae family, and specifically Frod, in an ILC3-dependent manner, which in turn can displace SFB and decrease protective intestinal Th17 cells.

### Example 6. Commensal Th17 Cells Protect from Metabolic Syndrome by Regulating Intestinal Lipid Absorption

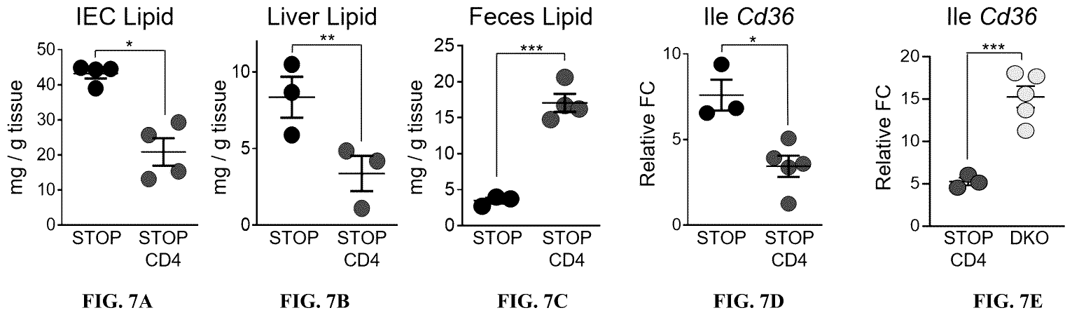

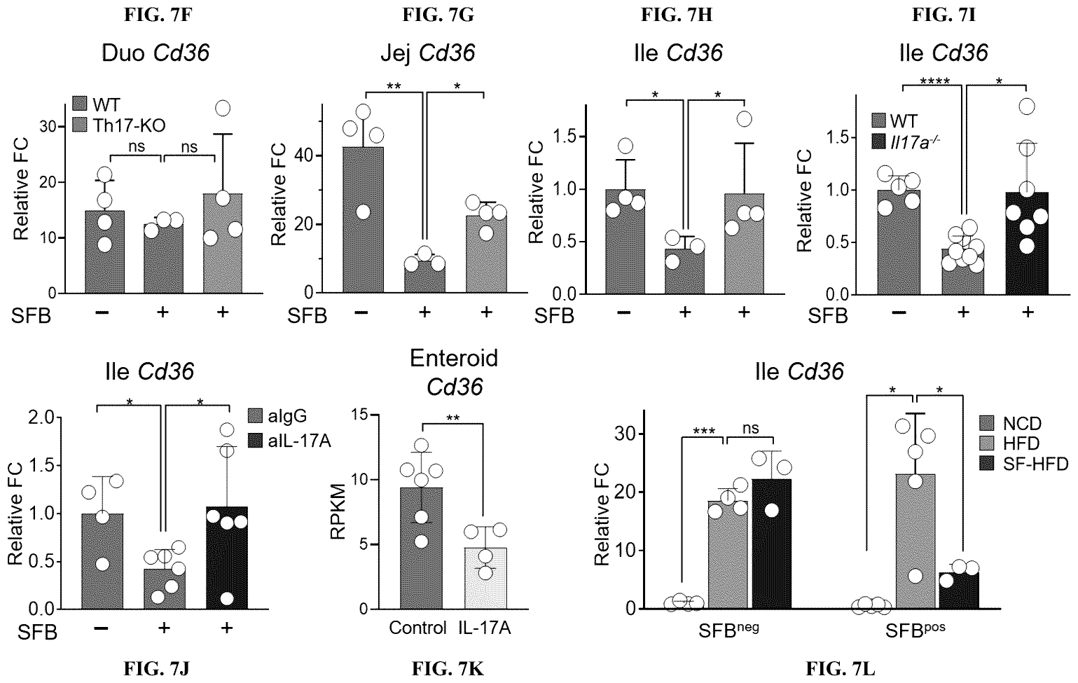

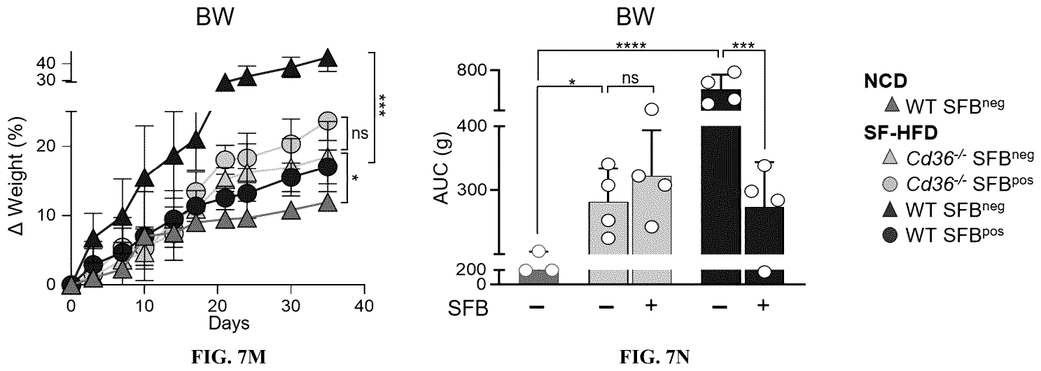

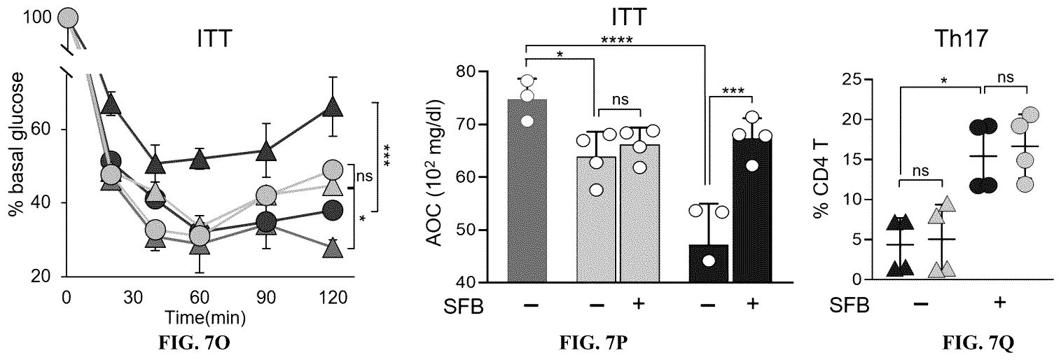

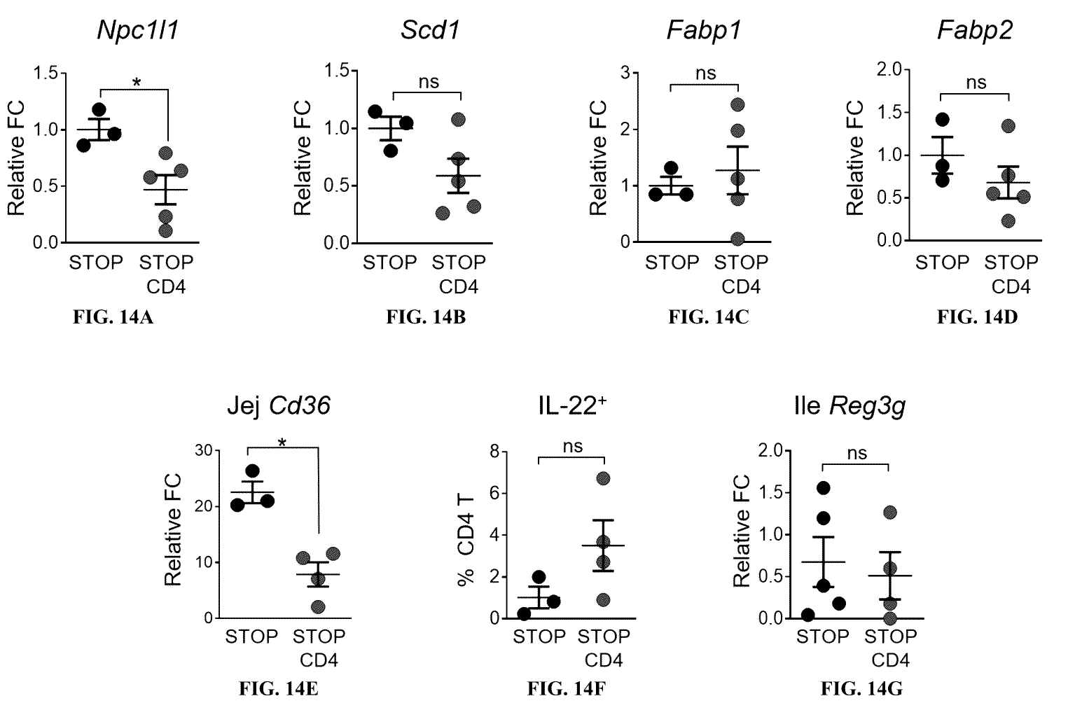

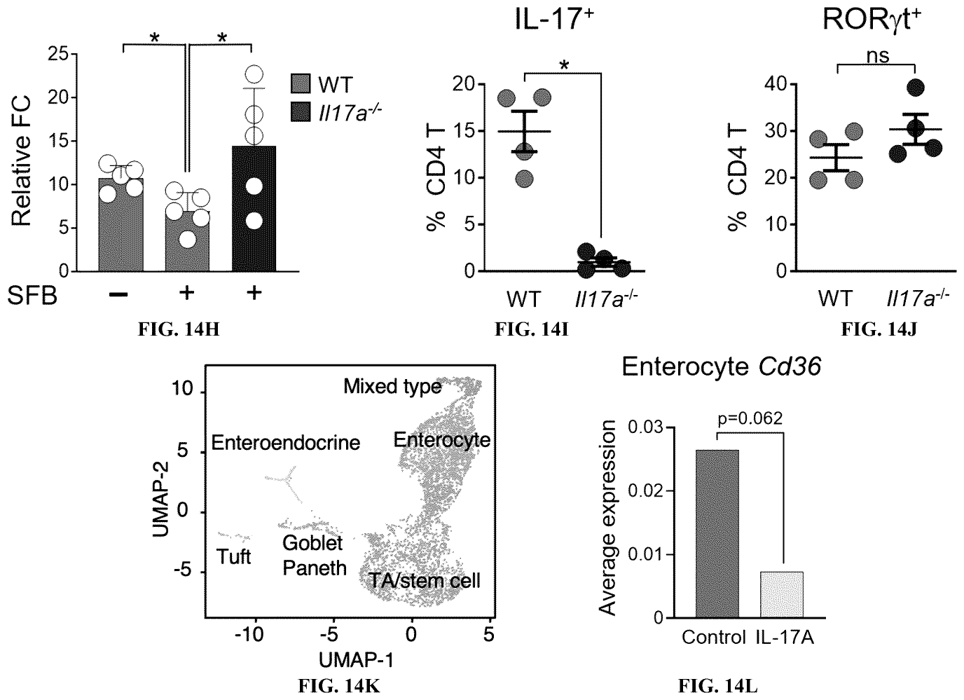

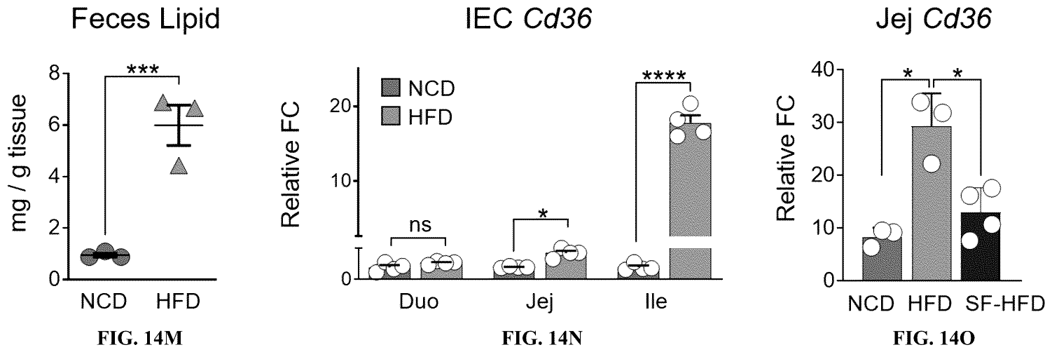

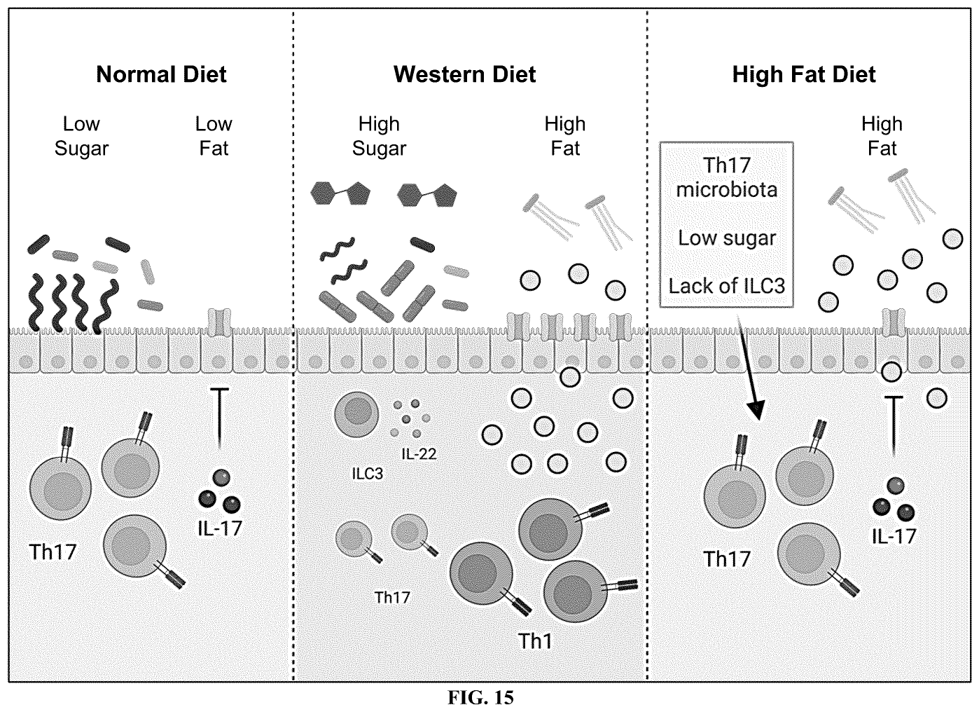

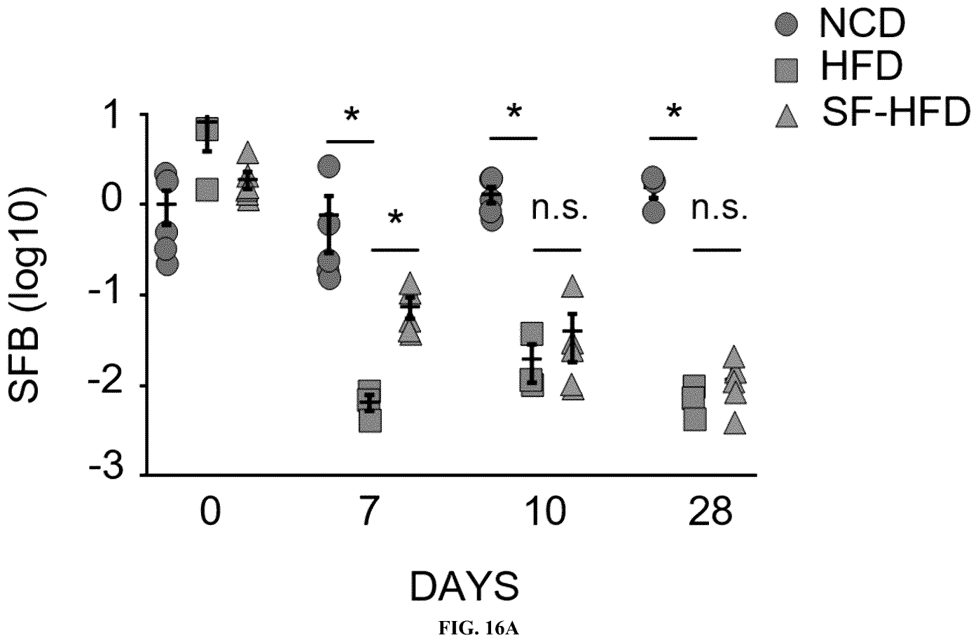

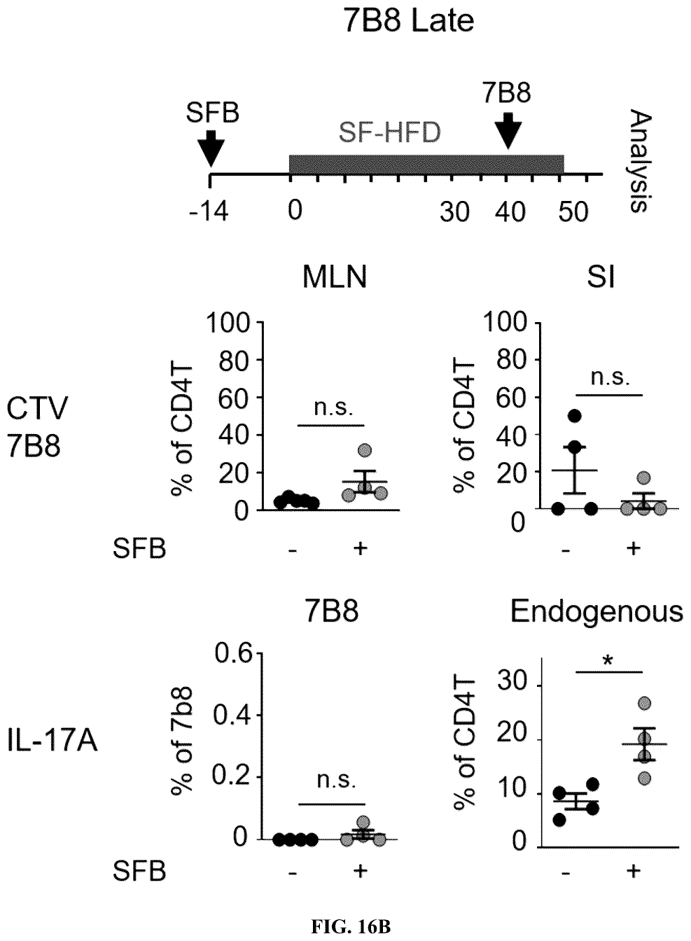

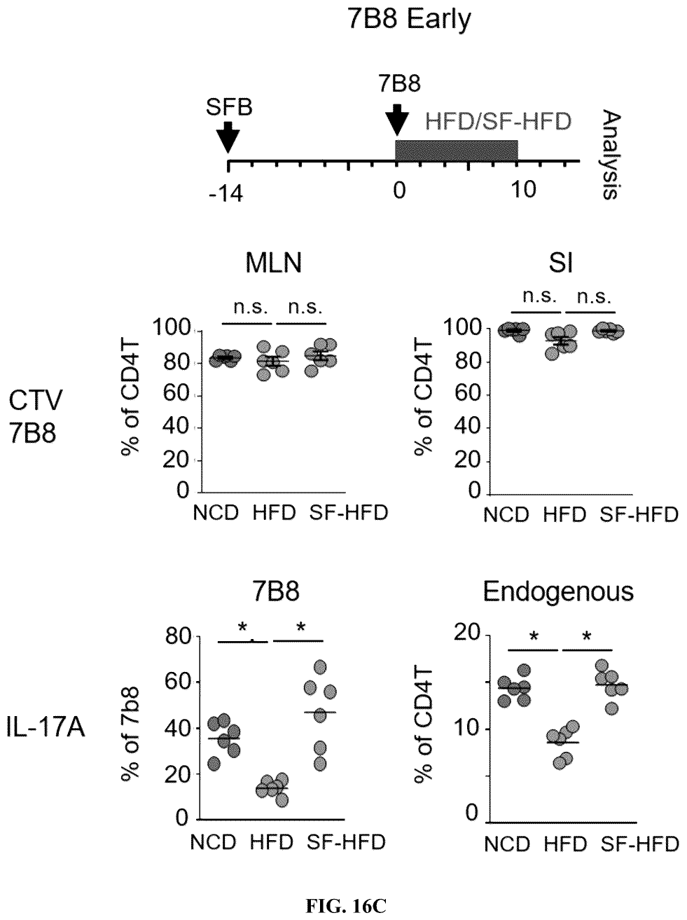

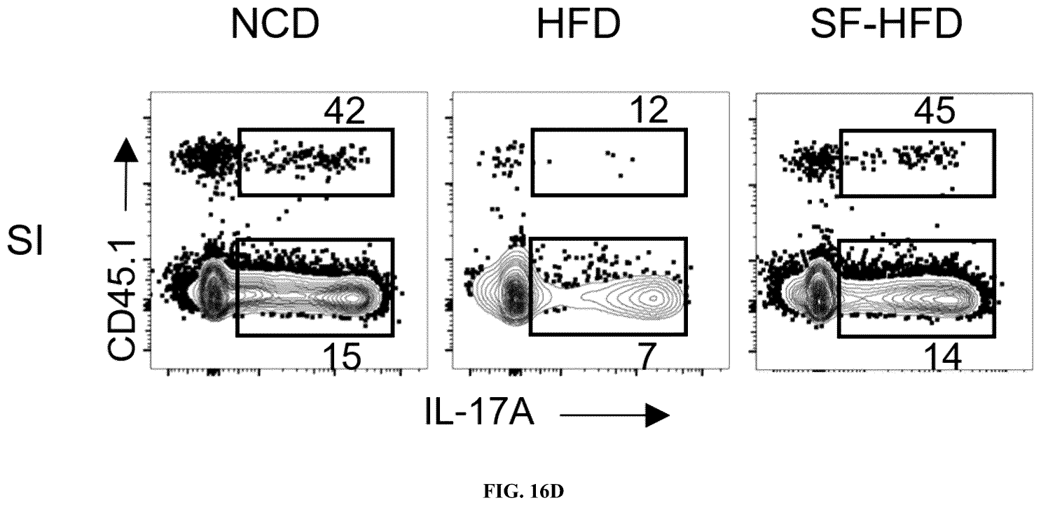

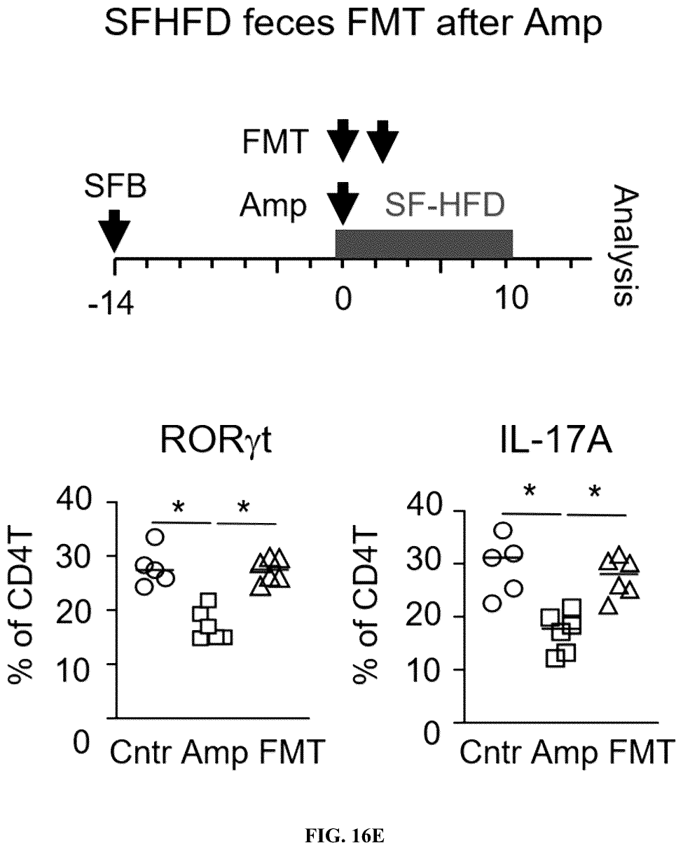

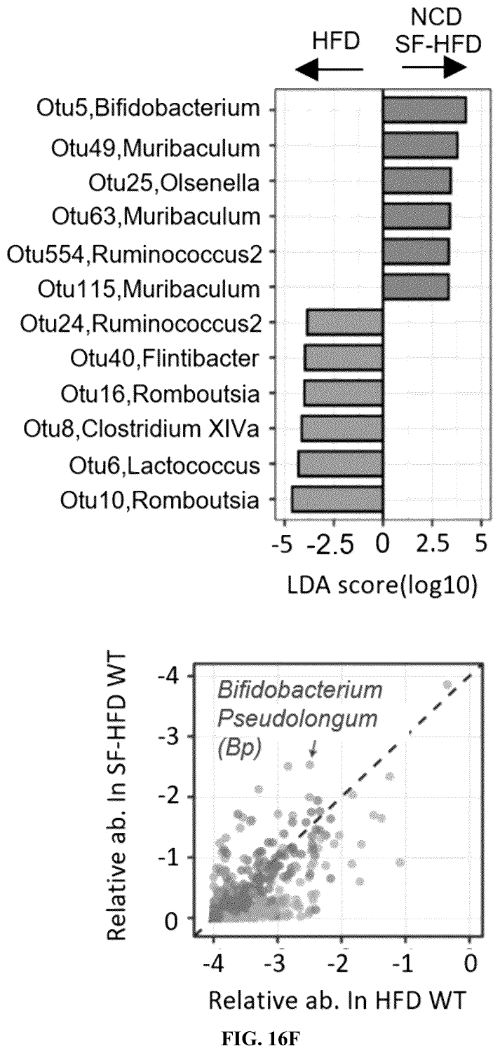

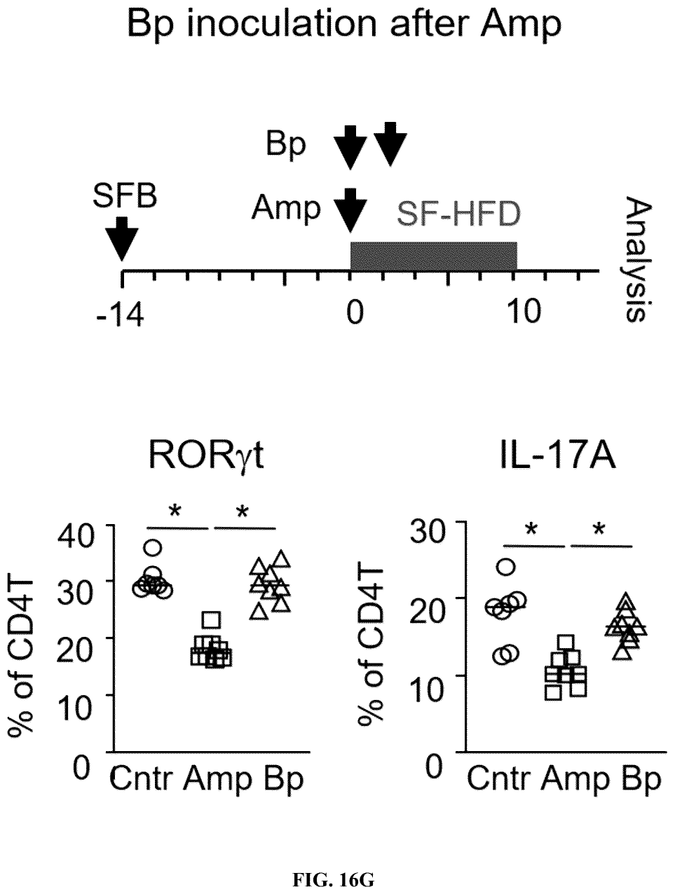

Next, the mechanism by which intestinal or commensal Th17 cells provide protection from metabolic syndrome was investigated. IL-17 has strong effects on intestinal epithelial cells (IEC) and maintains barrier integrity (Hueber et al., 2012; Lee et al., 2015; O'Connor et al., 2009). In the gut, epithelial absorption of dietary lipids is a known regulator of metabolic syndrome (Petersen et al., 2019; Wang et al., 2017). Therefore, the effects of commensal Th17 cells on intestinal lipid absorption was examined. Lipid concentrations in various tissues in HFD-fed Th17-deficient STOP mice that develop metabolic syndrome and Th17-sufficient STOP/CD4 mice, in which Th17 cells protect from metabolic syndrome were compared. STOP/CD4 mice had significantly decreased lipid content in IEC and liver (FIGS. 7A and 7B), but increased lipid levels in feces (FIG. 7C). This suggested that lipid absorption in IEC is reduced in the presence of Th17 cells, with concomitant accumulation of lipid in the gut lumen. Indeed, expression of several genes involved in lipid uptake and transport were downregulated in IEC of STOP/CD4 mice (FIGS. 7D and 14A-14D), most notably Cd36, encoding a transporter of dietary fatty acids into cells (Silverstein and Febbraio, 2009) (FIGS. 7D and 14E). Downregulation of CD36 in STOP/CD4 IEC required T cells insofar as it was not observed in IEC from HFD-fed αβ T cell-deficient STOP/CD4 mice (FIG. 7E). Moreover, CD36 downregulation was not mediated by IL-22, because both strains lack ILC3 and no difference in IL-22 production was detected from CD4 T cells (FIG. 14F) or in expression of IL-22 controlled genes in intestinal epithelium (FIG. 14G).

Epithelial CD36 has potent effects on dietary lipid absorption, thereby regulating metabolic syndrome (Nauli et al., 2006; Petersen et al., 2019; Wang et al., 2017). CD36 is highly expressed in the duodenum where lipid breakdown occurs, as well as jejunum, where most lipid absorption occurs, and its expression is lower in ileum at steady state (Chen et al., 2001) (FIGS. 7F-7H). SFB colonization specifically downregulates CD36 gene expression in distal SI (jejunum and ileum), but not in duodenum (FIGS. 7F-7H). CD36 downregulation was dependent on Th17 cell-derived IL-17, because it was not observed in Th17-deficient RORγt-flox/CD4-Cre mice (FIGS. 7F-7H), IL-17A-deficient mice (FIGS. 71 and 14H-14J), or WT animals treated with neutralizing anti-IL-17A antibody (FIG. 7J). Thus, intestinal or commensal Th17 cells can decrease lipid absorption in distal SI, by decreasing lipid uptake through CD36 in an IL-17-dependent manner. To investigate whether IL-17 acts directly on IEC we examined the effects of IL-17 on CD36 expression in SI enteroids in data from two independent published datasets. Analysis of bulk RNA-Seq data from (Kumar et al., 2016), revealed significant decrease of CD36 expression in terminal ileum enteroids treated with IL-17 in vitro (FIG. 7K). Analysis of single cell RNA-Seq data from total SI enteroids from (Biton et al., 2018), showed similar decrease of CD36 in SI enterocytes treated with IL-17 in vitro (FIGS. 14K and 14L), although the difference did not reach statistical significance. Together, these data suggest that Th17 cell-derived IL-17 can directly suppress CD36 expression in SI enterocytes.

At steady state, lipid absorption predominantly occurs in jejunum, however during DIO, and in HFD-fed animals, excess lipids carry over into the distal gut (FIG. 14M), and are taken up in the ileum (Buttet et al., 2016; de Wit et al., 2012). We found that HFD specifically and potently upregulates CD36 expression in terminal ileum (and to lesser extent in jejunum) (FIG. 14N). We hypothesized that sugar-sensitive commensal Th17 cells can protect by counteracting the upregulation of CD36. Indeed, both HFD and SF-HFD induced similar upregulation of CD36 in ileum and jejunum IEC in absence of SFB-specific Th17 cells (FIG. 7L). However, this upregulation was significantly suppressed in the presence of commensal Th17 cells in SF-HFD-fed animals (FIGS. 7L and 140).

To directly address whether the protective effects of Th17 cells require CD36 we fed SFB-negative and SFB-colonized WT and CD36-deficient animals SF-HFD. As earlier, SFB-induced Th17 cells provided protection from SF-HFD induced metabolic syndrome in WT animals (FIGS. 6M-7Q). CD36-deficient animals developed significantly less metabolic syndrome compared to WT animals, as previously reported (FIGS. 7M-7Q). However, in contrast to WT animals, SFB Th17 cells did not provide additional protection in the absence of CD36 (FIGS. 7M-7Q). The foregoing findings suggest that commensal Th17 cell-derived IL-17 decreases lipid uptake and absorption specifically in distal SI during DIO by controlling epithelial expression of the fatty acid transporter CD36.

### Example 7. STAR Methods Text

a. Resource Availability

### I. Data and Code Availability

16S-V4 rRNA sequencing data have been deposited at NCBI BioProject database and are publicly available as of the date of publication. Accession numbers are listed in the key resources table.

### Example 8. Experimental Model and Subject Details

a. Animals

Mice were purchased from the Jackson Laboratories and bred (except for Cd36^{-/-}mice) at Columbia University. Animals were purchased only from SFB-negative maximum barrier rooms at Jackson. All animals were tested for SFB upon arrival and maintained in an SFB-negative high barrier room at Columbia University. 7B8 mice were bred to CD45.1 and IL-17-GFP mice at Columbia University to generate 7B8.CD45.1.IL-17-GFP animals. RORγ-STOP mice were generated by homologous recombination in C57BL/6 ES cells.

The targeting vector generated an inversion of the Rorc genomic sequence containing Exons 3-6 surrounded by two pairs of LoxP and LoxP2272 sequences in opposite orientation in intron 2 and intron 6 (FIG. 9A-9H). To generate ILC3-deficient mice that can generate CD4 T cells and Th17 cells, RORγ-STOP mice were crossed to Cd4-Cre mice. All mice were bred and housed under high-barrier specific pathogen-free conditions at Columbia University Medical Center. Except for Cd364^{-/-}, all other lines were bred to heterozygosity and experiments were performed with littermate controls.

Whenever possible all genotypes were housed in the same cage to control for microbiota and cage effects. Cd36^{-/-} animals were ordered from the Jackson Laboratories with age and sex-matched C57BL/6J controls from the same room at Jackson and co-housed for two weeks prior to the start of the experiment and for the duration of the experiment to control for microbiota differences. Germ-free C57BL/6 mice were generated at the gnotobiotic facilities at Rockefeller University, Weill Cornell or Keio University and following defined flora colonization were housed in Techniplast isocages at the same institution. Metabolic experiments used 5-week-old males.

b. Diets

All diets were irradiated for sterility. Diets were provided in pellet form ad libitum and fresh diet provided twice a week. For 50:50 NCD:HFD mix, pellets from individual diets were mechanically disrupted into fine powder. 30 mg powder from each diet were mixed manually with 5-10 ml of autoclaved water to form a palatable pellet. The pellets were dried for 30 mins and provided ad libitum to the animals on a clean plastic plate inside the cage. Fresh mixed diet pellets were provided twice weekly. Individual diet formulations are listed in Supplemental Table 1.

c. Metabolic Measurements

5-week-old males were colonized with SFB on Day-14 and kept on NCD. SFB colonization was confirmed on Day-10 and Day-1. On Day-0 the animals were switched to corresponding diets. Body weight was followed every 3-4 days throughout the experiment. Insulin tolerance test was performed on Day-28. Oral glucose tolerance test was performed on Day-32. Tissue and cell isolation for flow cytometry or quantitative RT-PCR was performed on Day-35. For insulin tolerance test, animals were subjected to a 6-hour fast (8 AM-2 PM), followed by intraperitoneal insulin injection (0.75 U/kg, human insulin). Blood samples were obtained at 0, 20, 40, 60 and 120 min. For glucose tolerance test, animals were subjected to an overnight fast (6 PM-6 AM) followed by oral glucose gavage (1.2 g/kg of 12% dextrose solution). 2 μl blood samples were obtained at 0, 15, 30, 60 and 120 min.

d. T Cell and Cytokine Depletion In Vivo

To deplete CD4 T cells in vivo, animals were injected intraperitoneally with an anti-CD4 antibody or isotype control twice weekly starting on Day 0. To neutralize IL-17A, 300 ug/mouse of an IL-17A neutralizing antibody or IgG1 isotype control were injected intraperitoneally every other day.

e. Adoptive T Cell Transfers

For adoptive transfer experiments, 0.5 million CD4 T cells (95%-98% purity) were MACS-purified from spleens and lymph nodes of SFB-negative 7B8/CD45.1/IL-17A-GFP reporter mice, labeled with Cell Trace Violet proliferation dye (Life Technologies) and transferred intravenously into congenic CD45.2 WT mice fed with corresponding diets. Priming and IL-17A induction in SI LP were investigated 7 days after transfer.

For CD4 T cell reconstitution experiments, 5-10 million MACS purified CD4 T cells from spleens and LNs of SFB-negative WT/CD45.1/IL-17A-GFP reporter mice were transferred into recipient congenic STOP/CD45.2 mice.

f. Isolation of Tissue for RNA Preparation and Quantitative RT-PCR

RNA was isolated from tissues after homogenization in Trizol using manufacturer's protocol. After cDNA synthesis, Q-PCR was performed on LightCycler 480 (Roche) using SYBR Green. Individual parts of the intestine were prepared as follows. Duodenum was dissected macroscopically. The remaining small intestine was divided into four equal parts and the parts were defined as proximal jejunum, distal jejunum, proximal ileum and distal ileum. For RNA isolation 0.5 cm tissue was collected from the center of the pieces defined as duodenum (Duo), distal jejunum (Jej) and distal ileum (Ile). Intestinal epithelial cells (IECs) were collected from the corresponding part of the small intestine by thorough washing of mucus with PBS followed by gentle scraping of the luminal surface with a glass slide.

g. Lamina Propria Lymphocyte Isolation

LP lymphocytes isolation and intracellular cytokine and transcription factor staining were performed as described previously (Goto et al., 2014).

h. Adipose Tissue Immune Cell Isolation

Both flaps of epididymal fat were dissected and cut into small pieces. The sample was digested for 45 min at 37C° with 2 mg/ml type | collagenase and 1.5% FCS in RPMI.

i. Neutral Lipid Measurement

Neutral lipids were extracted using published protocol (Daniel K et al. Bio Protoc. 2015) and measured with a commercial lipid quantification kit (see Key Resources Table).

j. Public RNA-Seq Data Analysis

To examine if IL-17A acts on epithelial cells, previously reported RNA sequencing (RNA-seq) of mouse ileum enteroids (Kumar et al., 2016) and single-cell RNA sequencing (scRNA-seq) of total small intestine organoids (Biton et al., 2018) treated in vitro with rIL-17A or control were downloaded from NCBI Sequence Read Archive (SRA). For bulk RNA-seq, single-ended raw reads were processed by Cutadapt v2.1 (reference) with following parameters “--minimum-length 24-u 10--trim-n-q 15” to remove low-quality bases and Illumina adapters. Reads passing quality filtering were then aligned against mouse cDNA reference GRCm38 by Bowtie2 v2.3.4 (Langmead and Salzberg, 2012) in--very-sensitive mode and reads counts per gene were then normalized by gene length and sequencing depth, i.e., reads per kilobase per million mapped reads (RPKM), for expression level quantification. For scRNAseq, raw reads were processed by 10× Genomics Cellranger toolkit v1.0.1 according to the original paper.

Next, cells with fewer than 500 detected genes were excluded, and remaining cells were subjected to UMAP ordination and clustering following standard Seurat pipeline (Hao et al., 2021). Cell types were manually identified by top cluster markers and gene expression level was calculated by function “AverageExpression” implemented in Seurat package.

k. Bacterial Strains

SFB were obtained from feces of SFB-monocolonized mice housed at Keio University. Frod (PB1) and Bpl (1B11) were isolated in the Kenya Honda laboratory (RIKEN IMS) as previously described (Atarashi et al., 2015; Zagato et al., 2020). Bpl was chosen as a control strain for Frod gnotobiotic experiments, because it is a relatively high abundance species in our mouse colony that further increased after treatment with sucrose, but not with HFD. Frod and Bpl cultures were grown in pre-reduced Eggerth-Gagnon (EG) medium (Frod) or Reinforced Clostridial Medium (Fisher DF1808-17-3) (Bpl) in an anaerobic chamber (5% H₂, 10% CO₂, 85% N₂) at 37 C for 48 hours. For Frod colonization, mice were gavaged with 200 μl of overnight culture resuspended in pre-reduced PBS (optical density (OD) 600≅0.6; corresponding to about 5×10⁷ cfu/ml. For colonization of SPF mice, WT or STOP/CD4 recipient animals were pre-treated with 1 g/L Ampicillin in the drinking water for four days. For Bpl colonization, animals received 1×10⁸ cfu/ml of 1B11 in 200 μl pre-reduced PBS by oral gavage.

l. SFB Colonization and Relative SFB Quantification

SFB colonization was performed by single oral gavage of fecal suspension from SFB-enriched mice as previously described (Farkas et al., 2015). For SFB probiotic treatment, animals were gavaged every other day. Control animals were gavaged with fecal suspensions from SFB-negative littermate controls. To control for variability in SFB levels in feces used for gavage, all gavages were performed with frozen stocks from a single batch of SFB-enriched feces. To generate SFB-enriched feces a single cohort of 10 adult SFB-negative maximum barrier NSG mice from The Jackson Laboratory were colonized with feces from SFB-monocolonized mice.

Fecal samples were obtained in the period of 8 weeks, tested for SFB by quantitative RT-PCR and frozen as batch aliquots at −80 C. Control SFB-negative feces were collected from a separate cohort of littermate NSG mice housed in a similar manner. SFB colonization levels were confirmed by qPCR and normalized to levels of total bacteria (UNI) as previously described (Farkas et al., 2015).

m. Absolute Quantification of Bacterial Species in Feces

Absolute levels of SFB, Frod, and Bpl were measured by quantitative RT-PCR and quantified as μg of DNA per gram feces using standard curves from mono-colonized mice (SFB) or in vitro culture (Frod, Bpl).

n. Fecal Bacterial DNA Extraction and 16S rRNA Amplicon Sequencing.

Genomic DNA from feces was extracted using a silica bead beating-based protocol as previously described (Farkas et al., 2015). 16S sequencing of the V4 region was performed utilizing a custom dual-indexing protocol, detailed fully in (Ji et al., 2019).

o. OTU Clustering and Absolute Abundance Calculation.

Raw sequencing reads of 16S-V4 amplicons were analyzed by USEARCH v11.0.667 (Edgar, 2010). Specifically, paired-end reads were merged using “-fastq_mergepairs” mode with default setting. Merged reads were then subjected to quality filtering using “-fastq_filter” mode with the option “-fastq_maxee 1.0-fastq_minlen 240”. Remaining reads were deduplicated (-fastx_uniques) and clustered into OTUs (-unoise3) at 100% identity, and merged reads were then searched against OTU sequences (-otutab) to generate OTU count table. Taxonomy of OTUs were assigned using RDP classifier trained with 16S rRNA training set 18 (Wang et al., 2007). Sample total bacterial loads were calculated based on reads ratio of spike-in strain and sample weight, and relative abundance profiles of other taxa were then scaled by bacterial load to obtain absolute OTU abundances in arbitrary units, detailed fully in (Ji et al., 2019).

p. Public Human Feces Shotgun Metagenome Data Analysis.

To examine if Th17-inducing gut microbiota were altered under different conditions, two previously reported metagenome datasets from feces (metabolic syndrome versus healthy controls (Pedersen et al., 2016) and healthy individuals with recorded diet (Johnson et al., 2019)) were downloaded from NCBI Sequence Read Archive (SRA). Reference genomes of 20 Th17-inducing gut strains (Atarashi et al., 2015) were accessed according to the original paper and reference genomes of Bifidobacterium adolescentis and Eggerthella lenta were downloaded from NCBI Genome under accession NZ_CP028341.1 and NZ_CP089331.1. Reads of each sample were processed by Cutadapt v2.1 (Martin, 2011) with following parameters “--minimum-length 24:24-u 5-U 5-q 20--pair-filter-any” to remove low-quality bases and Nextera adapters.

Reads passing quality filtering were then aligned against reference genomes by Bowtie2 v2.3.4 (Langmead and Salzberg, 2012) in--very-sensitive mode and relative abundances of each strain were calculated as # of reads mapped to the strain normalized by total reads count. For the dataset of metabolic syndrome versus healthy control, 295 non-diabetic individuals were classified into “with metabolic syndrome” and “without metabolic syndrome” according to individual annotations in the original paper (Pedersen et al., 2016).

For the community of 20 Th17-inducing strains, high depletion was defined as more than 12 strains showing less than 25th percentile relative abundance across all individuals. For the dataset of recorded diet, index of “L2: sugar and sweets” in dietary records were extracted from the original paper (Johnson et al., 2019) and 509 samples from 34 individuals with accessible shotgun metagenome data were classified into high and low “Sugar and sweets consumption” using threshold of 25 for “L2: sugar and sweets” index.

q. Electron Microscopy

SFB-monocolonized mice were inoculated with Frod and samples from the terminal ileum were extracted after 24 hours and processed for electron microscopy as previously described (Ladinsky et al., 2019). Semi-thin (170 nm) sections were cut with a UC6 ultramicrotome (Leica Microsystems, Vienna), stained with uranyl acetate and lead citrate, and imaged on a Tecnai T12 transmission electron microscope (Thermo-Fisher Scientific) at 120k eV.

r. Quantification and Statistical Analysis

Statistical significance was determined by one-way ANNOVA for panels where more than three groups are being compared, and unpaired t test with Welch's correction for panels where two groups are compared. P values are represented on figures as follows: ns, not significant, *p<0.05, **p<0.01, ***p<0.005, ****p<0.001, *****p<0.0005. On time-course panels, p values with a bracket line connecting two curves refer to the whole curves and p values on top of individual datapoints refer only to those individual datapoints. Error bars on all figures represent standard deviation of the mean. Statistical analysis was performed using GraphPad Prism version 9.0 for Windows (GraphPad Software).

s. Key Resources Table

| | | |
|---|---|---|
| REAGENT or RESOURCE | SOURCE | IDENTIFIER |

Antibodies

| | | |
|---|---|---|
| Rat monoclonal anti- | BD | #564736 |
| CD196(Ccr6) antibody, BV421 | | |
| American hamster anti | eBioscience | #11-0031-82 |
| CD3e, 145-2C11, FITC | | |
| Rat monoclonal anti | Tonbo | #25-0042-U100 |
| CD4, RM4-5, APCCy7 | | |
| Rat monoclunal anti | Tonbo | #75-0042-U100 |
| CD4, RM4-5, BV450 | | |
| CD4(L3T4) MicroBeads, | Miltenyi Biotec | #130-117-043 |
| mouse | | |
| Rat monoclonal | eBioscience | #12-0081-82 |
| anti-CD8α, 53-6.7, PE | | |
| Rat anti CD11b, | Tonbo | #25-0112-U100 |
| M1/70, APCCy7 | | |
| American Hamster monoclonal | eBioscience | #25-0114-82 |
| anti CD11c, N418, PECy7 | | |
| Rat monoclonal anti | Tonbo | #48-0242-82 |
| CD24, M1/69, Pacific Blue | | |
| Mouse monoclonal | Tonbo | #60-0453-U100 |
| anti CD45.1, A20, PE-Cy7 | | |
| Mouse monoclonal | Tonbo | #50-0454-U100 |
| anti CD45.2, 104, PE | | |
| Rat monoclonal anti | Tonbo | #103232 |
| CD45R/B220, RA3-6B2, | | |
| Alexa Fluor 700 | | |
| Rat monoclonal anti | Tonbo | #30-0452-U100 |
| CD45R/B220, | | |
| RA3-6B2, Biotin | | |
| Mouse monoclonal | Biolegend | #139305 |
| anti CD64, X54-5/7.1, APC | | |
| Rat monoclonal anti | Biolegend | #141703 |
| CD206, C068C2, FITC | | |
| American Hamster | eBioscience | #17-5711-82 |
| monoclonal anti-gδTCR, | | |
| GL-3,GL3 APC | | |
| Rat monoclonal anti | Tonbo | #20-4801-U100 |
| F4/80, BM8.1, APC | | |
| Mouse monoclonal | eBioscience | #11-5773-82 |
| anti FoxP3, 3G3, FITC | | |
| Fixable Viability | Invitrogen | #65-0866-14 |
| Dye eFluor 506 (FVD) | | |
| Rat monoclonal anti- | eBioscience | #17-7311-82 |
| mouse IFNγ, XMG1.2, APC | | |
| Rat monoclonal anti-mouse | eBioscience | #12-7177-81 |
| IL-17A, eBio17B7, PE | | |
| Goat monoclonal anti IL-22 | Biolegend | #516406 |
| antibody (POLY5164) | | |
| Rat monoclonal anti-mouse | Biolegend | #128032 |
| Ly6C. HK1.4. BV421 | | |
| Rat monoclonal anti mouse | Tonbo | #60-1276-U100 |
| Ly6G, 1A8, PE-Cy7 | | |
| Rat monoclunal anti mouse | Tonbo | #80-5321-U100 |
| MHCII, M5/114.15.2, Alexa | | |
| Fluor 710 | | |
| Rat monoclonal anti-mouse | eBioscience | #46-3351-80 |
| NKp46, 29A1.4, PerCP- | | |
| Cyanine5.5 | | |
| Rat monoclonal anti- | eBioscience | #12-6988-82 |
| mouse RORγt, PE | | |
| Streptavidin PE-eFluor | eBioscience | #50-112-4814 |
| 610 conjugated | | |
| American Hamster | Biolegened | #47-0454-82 |
| anti-TCRβ, H57-597 | | |
| American Hamster | Tonbo | #65-5921-U100 |
| monoclonal TCRβ, H57-597, | | |
| PerCP-Cyanine5.5 | | |
| Rat monoclonal anti-Vβ14 | BD Bioscience | #553257 |
| TCR, 14-2(RUO), Biotin | | |

Bacterial and virus strains

| | | |
|---|---|---|
| Segmented Filamentous | Kenya Honda | (Umesaki |
| Bacteria (SFB) | | et al., 1995) |
| Faecalibaculum rodentium | Kenya Honda | (Zagato |
| (Frod) (PB1) | | et al., 2020) |
| Bifidobacterium pseudolongum | Kenya Honda | (Atarashi |
| (Bpl) (1B11) | | et al., 2015) |

Chemicals, peptides, and recombinant proteins

| | | |
|---|---|---|
| In VivoMAb anti-mouse IL-17A | BioXcell, | #BE0173 |
| neutralizing antibody | | |
| (clone 17F3) | | |
| In VivoMAb mouse IgG1 | BioXcell, | #BE0083 |
| isotype control (MOPC-21) | | |
| In VivoMAb anti mouse | BioXcell | #BE0003-1 |
| CD4 Antibody (clone GK1.5.) | | |
| In VivoMAb rat IgG2b | BioXcell | #BE0090 |
| isotype control, anti | | |
| keyhole limpet | | |
| hemocyanin (clone LTF-2) | | |
| Corning Dispase, 100 mL | Corning (Fisher) | #354235 |
| Roche Collagenase D 2.5 g | Roche (Sigma) | #11088882001 |
| from C. histolyticum | | |
| Collagenase, type 1, powder | Gibco | #17018209 |
| Deoxyribonuclease I from | Sigma | DN-25 |
| bovine pancreas, 1 g | | |
| Sucrose (Crystaline/Certified | Fisher Chemical | #207263 |
| ACS(12-500-500 g) | | |
| Glucose (G8270-1kg. ≥99.5%) | SIGMA | #080M0175V |
| Galactose (BP656-500 g) | Fisher bioreagents | #188103 |
| Maltodextrin (419680-100 g) | SIGMA-ALDRICH | #MKCL0273 |
| DE13.0-17.0 | | |
| Dextrose (D16-500, 500 mg) | Fisher Scientific | #D16-500 |
| Hanks′ Balanced Salt | CORNING | #36320020 |
| solution (HBSS), 10X | | |
| HyClone ™ RPMI 1640 | Cytiva | SH30028.LS |
| Medium, Sterile, pH 7.0-7.4, | | |
| With L-glutamine, Liquid | | |
| Sodium Bicarbonate | SIGMA | #46H02825 |
| Percoll ®, Sterile, | Cytiva | 17-0891-09 |
| pH 8.5-9.5, Liquid | | |
| Fetal Bovine Serum, | Thermo Scientific | #10437028 |
| Qualified, USDA approved | | |
| HEPES(1M) | Thermofisher | #15630-080 |
| Insulin human (recombinant | Roche | #11376497001 |
| yeast, 100 mg) | | |
| Fisher Chemical BP176025 | Fisher Scientific | #176025 |
| Ampicillin Sodium Salt | | |
| Invitrogen ambion Sodium | Fisher scientific | #AM9740 |
| Acetate (3M), pH 5.5 | | |
| Phenol/Chloroform/Isoamyl | Fisher Scientific | 327115000 |
| alcohol (25:24:1), stabilized | | |
| Ambion TRIzol reagent | Fisher Scientific | 15-596-018 |
| 2-Propanol, ACS | Sigma-Aldrich | #190764 |
| reagent, ≥99.5% | | |
| Proteinase K | Lucigen | #MPRK092 |
| Cell Trace Violet cell | Life Technologies | #34557 |
| proliferation kit | | |
| Ionomycin calcium | Sigma-Aldrich | #10634 |
| salt from Streptomyces | | |
| PMA, for use in | Sigma-Aldrich | #P1585 |
| molecular biology | | |
| Brefeldin A, from Penicillium | Sigma Aldrich | #B7651-5MG |
| brefeldianum, ≥99% (HPLC | | |
| and TLC) | | |
| BD cytofix/cytoperm | Fisher scientific | #BDB554722 |
| Fixation and Permeabilization | | |
| BD Perm/Wash Buffer | Fisher scientific | #BD554723 |
| Foxp3/Transcription Factor | TONBO | #TNB-1020-L050 |
| Fix/Perm Concentrate (4X) | Biosciences | |
| Foxp3/Transcription | TONBO | #TNB-0607-KIT |
| Factor Staining Buffer Kit | Biosciences | |
| Flow Cytometry | TONBO | #TNB-1213-L150 |
| Perm Buffer (10X) | Biosciences | |

Critical commercial assays

| | | |
|---|---|---|
| Neutral Lipid quantification, | Fisher cell Bio Lab | STA-617 |
| Fluorometric | | |
| Fluorometric Reagents | Fisher cell Bio Lab | #261701 |
| (100x) 1 vial 200 μL | | |
| Lipid standard, 1 vial | Fisher cell Bio Lab | #261702 |
| 100 μL, 20 g/dl | | |
| Qscript cDNA Super | VwR | #101414-108 |
| Mix, QuntaBio | | |
| 2X Universal SYBR | ABclonal | #RK21203 |
| Green Fast qPCR Mix-25 mL | | |
| Qubit dsDNA HS assay kit | ThermoFisher | #Q32581 |

Deposited data

| | | |
|---|---|---|
| 16S-V4 rRNA sequencing | This Study | accession ID |
| data NCBI BioProject database | | PRJNA844056 |
| (http://www.ncbi.nlm.nih.gov/ | | |
| bioproject/) | | |

Experimental models: Organisms/strains

| | | |
|---|---|---|
| C57BL/6J, Room RB15 | The Jackson | #000664 |
| | Laboratory | |
| Ptprc (CD45.1) | The Jackson | #002014 |
| | Laboratory | |
| Cd4-Cre | The Jackson | #022071 |
| | Laboratory | |
| 7B8 TCR Tg | The Jackson | #027230 |
| | Laboratory | |
| RORγ-flox | The Jackson | #008771 |
| | Laboratory | |
| IL-17A-GFP | The Jackson | #018472 |
| | Laboratory | |
| Il17a^{-/-} | The Jackson | #016879 |
| | Laboratory | |
| Cd36^{-/-} | The Jackson | #019006 |
| | Laboratory | |
| Tcrβ^{-/-} | The Jackson | #002118 |
| | Laboratory | |
| RORγ-STOP mice | This Study | N/A |

Software and algorithms

| | | |
|---|---|---|
| Flow jo_v10.6.2 | BD | N/A |
| Cutadapt v2.1 | N/A | N/A |
| Bowtie2 v2.3.4 | N/A | N/A |
| 10X Genomics Cellranger | N/A | N/A |
| toolkit v1.0.1 | | |
| USEARCH v11.0.667 | N/A | N/A |
| RDP classifier trained | N/A | N/A |
| with 16S rRNA training set 18 | | |

Other

| | | |
|---|---|---|
| BD LSR Fortessa | BD | N/A |
| Flow Cytometer | | |
| Irradiated Normal | Lab Diet | #5061 |
| Chow Diet, Lab diet 5061 | | |
| (NCD, Pico-Vac | | |
| Rodent Diet 20) | | |
| Irradiated High Fat Diet (HFD) | Research Diets | #D12492i |
| Irradiated Sugar Free | Research Diets | #D19122401i |
| Starch Add High Fat Diet | | |
| (SF-HFD) | | |
| Irradiated Inulin supplemented | Research Diets | #D13081106i |
| High Fat Diet (HFD + Inulin) | | |
| Irradiated Low Fat Diet (LFD) | Research Diets | #D12450Ji |
| Zirconia/Silica Beads 0.1 mm | Fisher Scientific | #11079101z |
| Miltenyi Biotec, Inc. | Miltenyil Biotec | #130-042-401 |
| LS Columns 25/PK | | |
| LightCycler ® 480 System | Roche | N/A |
| Fisherbrand Razor Blades | Fisher Schientific | #12640 |
| Insulin Syringes with | BD | #329420 |
| Permanently Attached Needles | | |
| Cell Strainer, Individual | VWR | #76327-098 |
| Package, 40 um, blue | | |
| HPM-010 high-pressure | Leica | N/A |
| freezing machine | Microsystems, | |
| | Vienna Austria | |
| 20% PhiX spike-in | Illumina | FC-110-3001 |
| (Illumina FC-110-3001) | | |
| Bead beater | Biospec | #1001 |
| Beads cleanup | Beckman-Coulter | # A63881 |
| AFS-2 freeze-substitution | Leica Microsystems | N/A |
| machine | | |
| UC6 ultramicrotome | Leica Microsystems | N/A |
| Tecnai TF30ST-FEG | Gatan Inc., | N/A |
| transmission electron | Pleasanton | |
| microscope (300 KeV) | CA | |
| equipped with a | | |
| 2k x 2k CCD camera (XP1000). | | |

### REFERENCES

ADDIN EN.REFLIST Afshin, A., Forouzanfar, M. H., Reitsma, M. B., Sur, P., Estep, K., Lee, A., Marczak, L., Mokdad, A. H., Moradi-Lakeh, M., Naghavi, M., et al. (2017). Health Effects of Overweight and Obesity in 195 Countries over 25 Years. The New England journal of medicine 377, 13-27.
Alexander, M., Ang, Q. Y., Nayak, R. R., Bustion, A. E., Sandy, M., Zhang, B., Upadhyay, V., Pollard, K. S., Lynch, S. V., and Turnbaugh, P. J. (2022). Human gut bacterial metabolism drives Th17 activation and colitis. Cell host & microbe 30, 17-30 e19.
Atarashi, K., Nishimura, J., Shima, T., Umesaki, Y., Yamamoto, M., Onoue, M., Yagita, H., Ishii, N., Evans, R., Honda, K., et al. (2008). ATP drives lamina propria T (H) 17 cell differentiation. Nature 455, 808-812.
Atarashi, K., Tanoue, T., Ando, M., Kamada, N., Nagano, Y., Narushima, S., Suda, W., Imaoka, A., Setoyama, H., Nagamori, T., et al. (2015). Th17 Cell Induction by Adhesion of Microbes to Intestinal Epithelial Cells. Cell 163, 367-380.
Barman, M., Unold, D., Shifley, K., Amir, E., Hung, K., Bos, N., and Salzman, N. (2008). Enteric salmonellosis disrupts the microbial ecology of the murine gastrointestinal tract. Infect Immun 76, 907-915.
Basson, A. R., Chen, C., Sagl, F., Trotter, A., Bederman, I., Gomez-Nguyen, A., Sundrud, M. S., Ilic, S., Cominelli, F., and Rodriguez-Palacios, A. (2020). Regulation of Intestinal Inflammation by Dietary Fats. Front Immunol 11, 604989.
Biton, M., Haber, A. L., Rogel, N., Burgin, G., Beyaz, S., Schnell, A., Ashenberg, O., Su, C. W., Smillie, C., Shekhar, K., et al. (2018). T Helper Cell Cytokines Modulate Intestinal Stem Cell Renewal and Differentiation. Cell 175, 1307-1320 e1322.
Bravo, S., Lowndes, J., Sinnett, S., Yu, Z., and Rippe, J. (2013). Consumption of sucrose and high-fructose corn syrup does not increase liver fat or ectopic fat deposition in muscles. Appl Physiol Nutr Metab 38, 681-688.
Buttet, M., Poirier, H., Traynard, V., Gaire, K., Tran, T. T., Sundaresan, S., Besnard, P., Abumrad, N. A., and Niot, I. (2016). Deregulated Lipid Sensing by Intestinal CD36 in Diet-Induced Hyperinsulinemic Obese Mouse Model. PloS one 11, e0145626.
Cai, L., Wang, Z., Ji, A., Meyer, J. M., and van der Westhuyzen, D. R. (2012). Scavenger receptor CD36 expression contributes to adipose tissue inflammation and cell death in diet-induced obesity. PloS one 7, e36785.
Cani, P. D., Bibiloni, R., Knauf, C., Waget, A., Neyrinck, A. M., Delzenne, N. M., and Burcelin, R. (2008). Changes in gut microbiota control metabolic endotoxemia-induced inflammation in high-fat diet-induced obesity and diabetes in mice. Diabetes 57, 1470-1481.
Chen, M., Yang, Y., Braunstein, E., Georgeson, K. E., and Harmon, C. M. (2001). Gut expression and regulation of FAT/CD36: possible role in fatty acid transport in rat enterocytes. Am J Physiol Endocrinol Metab 281, E916-923.
Chen, Y., Zhang, J., Cui, W., and Silverstein, R. L. (2022). CD36, a signaling receptor and fatty acid transporter that regulates immune cell metabolism and fate. The Journal of experimental medicine 219.
Choi, G. B., Yim, Y. S., Wong, H., Kim, S., Kim, H., Kim, S. V., Hoeffer, C. A., Littman, D. R., and Huh, J. R. (2016). The maternal interleukin-17a pathway in mice promotes autism-like phenotypes in offspring. Science 351, 933-939.
Dalmas, E., Venteclef, N., Caer, C., Poitou, C., Cremer, I., Aron-Wisnewsky, J., Lacroix-Desmazes, S., Bayry, J., Kaveri, S. V., Clément, K., et al. (2014). T cell-derived IL-22 amplifies IL-1β-driven inflammation in human adipose tissue: relevance to obesity and type 2 diabetes. Diabetes 63, 1966-1977.
de Wit, N., Derrien, M., Bosch-Vermeulen, H., Oosterink, E., Keshtkar, S., Duval, C., de Vogel-van den Bosch, J., Kleerebezem, M., Muller, M., and van der Meer, R. (2012). Saturated fat stimulates obesity and hepatic steatosis and affects gut microbiota composition by an enhanced overflow of dietary fat to the distal intestine. American journal of physiology Gastrointestinal and liver physiology 303, G589-599.
Edgar, R. C. (2010). Search and clustering orders of magnitude faster than BLAST. Bioinformatics 26, 2460-2461.
Fabbrini, E., Cella, M., McCartney, S. A., Fuchs, A., Abumrad, N. A., Pietka, T. A., Chen, Z., Finck, B. N., Han, D. H., Magkos, F., et al. (2013). Association between specific adipose tissue CD4+ T-cell populations and insulin resistance in obese individuals. Gastroenterology 145, 366-374.e361-363.
Farkas, A. M., Panea, C., Goto, Y., Nakato, G., Galan-Diez, M., Narushima, S., Honda, K., and Ivanov, I I (2015). Induction of Th17 cells by segmented filamentous bacteria in the murine intestine. J Immunol Methods 421, 104-111.
Febbraio, M., Abumrad, N. A., Hajjar, D. P., Sharma, K., Cheng, W., Pearce, S. F., and Silverstein, R. L. (1999). A null mutation in murine CD36 reveals an important role in fatty acid and lipoprotein metabolism. The Journal of biological chemistry 274, 19055-19062.
Garidou, L., Pomie, C., Klopp, P., Waget, A., Charpentier, J., Aloulou, M., Giry, A., Serino, M., Stenman, L., Lahtinen, S., et al. (2015). The Gut Microbiota Regulates Intestinal CD4 T Cells Expressing RORgammat and Controls Metabolic Disease. Cell Metab 22, 100-112.
Global, B. M. I. M. C., Di Angelantonio, E., Bhupathiraju Sh, N., Wormser, D., Gao, P., Kaptoge, S., Berrington de Gonzalez, A., Cairns, B. J., Huxley, R., Jackson Ch, L., et al. (2016). Body-mass index and all-cause mortality: individual-participant-data meta-analysis of 239 prospective studies in four continents. Lancet 388, 776-786.
Goto, Y., Panea, C., Nakato, G., Cebula, A., Lee, C., Diez, M. G., Laufer, T. M., Ignatowicz, L., and Ivanov, II (2014). Segmented filamentous bacteria antigens presented by intestinal dendritic cells drive mucosal Th17 cell differentiation. Immunity 40, 594-607.
Hajri, T., Hall, A. M., Jensen, D. R., Pietka, T. A., Drover, V. A., Tao, H., Eckel, R., and Abumrad, N. A. (2007). CD36-facilitated fatty acid uptake inhibits leptin production and signaling in adipose tissue. Diabetes 56, 1872-1880.
Hao, Y., Hao, S., Andersen-Nissen, E., Mauck, W. M., 3rd, Zheng, S., Butler, A., Lee, M. J., Wilk, A. J., Darby, C., Zager, M., et al. (2021). Integrated analysis of multimodal single-cell data. Cell 184, 3573-3587 e3529.
Honda, K., and Littman, D. R. (2016). The microbiota in adaptive immune homeostasis and disease. Nature 535, 75-84.
Hong, C. P., Park, A., Yang, B. G., Yun, C. H., Kwak, M. J., Lee, G. W., Kim, J. H., Jang, M. S., Lee, E. J., Jeun, E. J., et al. (2017). Gut-Specific Delivery of T-Helper 17 Cells Reduces Obesity and Insulin Resistance in Mice. Gastroenterology 152, 1998-2010.
Hueber, W., Sands, B. E., Lewitzky, S., Vandemeulebroecke, M., Reinisch, W., Higgins, P. D., Wehkamp, J., Feagan, B. G., Yao, M. D., Karczewski, M., et al. (2012). Secukinumab, a human anti-IL-17A monoclonal antibody, for moderate to severe Crohn's disease: unexpected results of a randomised, double-blind placebo-controlled trial. Gut 61, 1693-1700.
Ivanov, II, Atarashi, K., Manel, N., Brodie, E. L., Shima, T., Karaoz, U., Wei, D., Goldfarb, K. C., Santee, C. A., Lynch, S. V., et al. (2009). Induction of intestinal Th17 cells by segmented filamentous bacteria. Cell 139, 485-498.
Ivanov, II, Tuganbaev, T., Skelly, A. N., and Honda, K. (2022). T Cell Responses to the Microbiota. Annual review of immunology.
Ji, B. W., Sheth, R. U., Dixit, P. D., Huang, Y., Kaufman, A., Wang, H. H., and Vitkup, D. (2019). Quantifying spatiotemporal variability and noise in absolute microbiota abundances using replicate sampling. Nat Methods 16, 731-736.
Johnson, A. J., Vangay, P., Al-Ghalith, G. A., Hillmann, B. M., Ward, T. L., Shields-Cutler, R. R., Kim, A. D., Shmagel, A. K., Syed, A. N., Walter, J., et al. (2019). Daily Sampling Reveals Personalized Diet-Microbiome Associations in Humans. Cell host & microbe 25, 789-802 e785.
Johnson, R. J., Nakagawa, T., Sanchez-Lozada, L. G., Shafiu, M., Sundaram, S., Le, M., Ishimoto, T., Sautin, Y. Y., and Lanaspa, M. A. (2013). Sugar, uric acid, and the etiology of diabetes and obesity. Diabetes 62, 3307-3315.
Kawano, Y., Nakae, J., Watanabe, N., Kikuchi, T., Tateya, S., Tamori, Y., Kaneko, M., Abe, T., Onodera, M., and Itoh, H. (2016). Colonic Pro-inflammatory Macrophages Cause Insulin Resistance in an Intestinal Ccl2/Ccr2-Dependent Manner. Cell Metab 24, 295-310.
Kennedy, D. J., and Kashyap, S. R. (2011). Pathogenic role of scavenger receptor CD36 in the metabolic syndrome and diabetes. Metab Syndr Relat Disord 9, 239-245.
Khan, S., Luck, H., Winer, S., and Winer, D. A. (2021). Emerging concepts in intestinal immune control of obesity-related metabolic disease. Nat Commun 12, 2598.
Klose, C. S., and Artis, D. (2016). Innate lymphoid cells as regulators of immunity, inflammation and tissue homeostasis. Nature immunology 17, 765-774.
Kumar, P., Monin, L., Castillo, P., Elsegeiny, W., Horne, W., Eddens, T., Vikram, A., Good, M., Schoenborn, A. A., Bibby, K., et al. (2016). Intestinal Interleukin-17 Receptor Signaling Mediates Reciprocal Control of the Gut Microbiota and Autoimmune Inflammation. Immunity 44, 659-671.
Ladinsky, M. S., Araujo, L. P., Zhang, X., Veltri, J., Galan-Diez, M., Soualhi, S., Lee, C., Irie, K., Pinker, E. Y., Narushima, S., et al. (2019). Endocytosis of commensal antigens by intestinal epithelial cells regulates mucosal T cell homeostasis. Science 363.
Laffin, M., Fedorak, R., Zalasky, A., Park, H., Gill, A., Agrawal, A., Keshteli, A., Hotte, N., and Madsen, K. L. (2019). A high-sugar diet rapidly enhances susceptibility to colitis via depletion of luminal short-chain fatty acids in mice. Sci Rep 9, 12294.
Langmead, B., and Salzberg, S. L. (2012). Fast gapped-read alignment with Bowtie 2. Nat Methods 9, 357-359.
Lee, J. S., Tato, C. M., Joyce-Shaikh, B., Gulen, M. F., Cayatte, C., Chen, Y., Blumenschein, W. M., Judo, M., Ayanoglu, G., McClanahan, T. K., et al. (2015). Interleukin-23-Independent IL-17 Production Regulates Intestinal Epithelial Permeability. Immunity 43, 727-738.
Ley, R. E., Turnbaugh, P. J., Klein, S., and Gordon, J. I. (2006). Microbial ecology: human gut microbes associated with obesity. Nature 444, 1022-1023.
Luck, H., Tsai, S., Chung, J., Clemente-Casares, X., Ghazarian, M., Revelo, X. S., Lei, H., Luk, C. T., Shi, S. Y., Surendra, A., et al. (2015). Regulation of obesity-related insulin resistance with gut anti-inflammatory agents. Cell Metab 21, 527-542.
Lugli, G. A., Duranti, S., Albert, K., Mancabelli, L., Napoli, S., Viappiani, A., Anzalone, R., Longhi, G., Milani, C., Turroni, F., et al. (2019). Unveiling Genomic Diversity among Members of the Species Bifidobacterium pseudolongum, a Widely Distributed Gut Commensal of the Animal Kingdom. Applied and environmental microbiology 85.
Macdonald, I. A. (2016). A review of recent evidence relating to sugars, insulin resistance and diabetes. Eur J Nutr 55, 17-23.
Malik, V. S., Popkin, B. M., Bray, G. A., Despres, J. P., and Hu, F. B. (2010). Sugar-sweetened beverages, obesity, type 2 diabetes mellitus, and cardiovascular disease risk. Circulation 121, 1356-1364. Martin, M. (2011). Cutadapt removes adapter sequences from high-throughput sequencing reads. EMBnetjournal 17, 10-12.
Nauli, A. M., Nassir, F., Zheng, S., Yang, Q., Lo, C. M., Vonlehmden, S. B., Lee, D., Jandacek, R. J., Abumrad, N. A., and Tso, P. (2006). CD36 is important for chylomicron formation and secretion and may mediate cholesterol uptake in the proximal intestine. Gastroenterology 131, 1197-1207.
O'Connor, W., Jr., Kamanaka, M., Booth, C. J., Town, T., Nakae, S., Iwakura, Y., Kolls, J. K., and Flavell, R. A. (2009). A protective function for interleukin 17A in T cell-mediated intestinal inflammation. Nature immunology 10, 603-609.
Omenetti, S., Bussi, C., Metidji, A., Iseppon, A., Lee, S., Tolaini, M., Li, Y., Kelly, G., Chakravarty, P., Shoaie, S., et al. (2019). The Intestine Harbors Functionally Distinct Homeostatic Tissue-Resident and Inflammatory Th17 Cells. Immunity 51, 77-89 e76.
Pedersen, H. K., Gudmundsdottir, V., Nielsen, H. B., Hyotylainen, T., Nielsen, T., Jensen, B. A., Forslund, K., Hildebrand, F., Prifti, E., Falony, G., et al. (2016). Human gut microbes impact host serum metabolome and insulin sensitivity. Nature 535, 376-381.
Pepino, M. Y., Kuda, O., Samovski, D., and Abumrad, N. A. (2014). Structure-function of CD36 and importance of fatty acid signal transduction in fat metabolism. Annual review of nutrition 34, 281-303.
Perez, M. M., Martins, L. M. S., Dias, M. S., Pereira, C. A., Leite, J. A., Goncalves, E. C. S., de Almeida, P. Z., de Freitas, E. N., Tostes, R. C., Ramos, S. G., et al. (2019). Interleukin-17/interleukin-17 receptor axis elicits intestinal neutrophil migration, restrains gut dysbiosis and lipopolysaccharide translocation in high-fat diet-induced metabolic syndrome model. Immunology 156, 339-355.
Petersen, C., Bell, R., Klag, K. A., Lee, S. H., Soto, R., Ghazaryan, A., Buhrke, K., Ekiz, H. A., Ost, K. S., Boudina, S., et al. (2019). T cell-mediated regulation of the microbiota protects against obesity. Science 365.
Racine, A., Carbonnel, F., Chan, S. S., Hart, A. R., Bueno-de-Mesquita, H. B., Oldenburg, B., van Schaik, F. D., Tjonneland, A., Olsen, A., Dahm, C. C., et al. (2016). Dietary Patterns and Risk of Inflammatory Bowel Disease in Europe: Results from the EPIC Study. Inflammatory bowel diseases 22, 345-354.
Sano, T., Huang, W., Hall, J. A., Yang, Y., Chen, A., Gavzy, S. J., Lee, J. Y., Ziel, J. W., Miraldi, E. R., Domingos, A. I., et al. (2015). An IL-23R/IL-22 Circuit Regulates Epithelial Serum Amyloid A to Promote Local Effector Th17 Responses. Cell 163, 381-393.
Sasaki, T., Moro, K., Kubota, T., Kubota, N., Kato, T., Ohno, H., Nakae, S., Saito, H., and Koyasu, S. (2019). Innate Lymphoid Cells in the Induction of Obesity. Cell reports 28, 202-217 e207.
Silverstein, R. L., and Febbraio, M. (2009). CD36, a scavenger receptor involved in immunity, metabolism, angiogenesis, and behavior. Sci Signal 2, re3.
Stanhope, K. L. (2016). Sugar consumption, metabolic disease and obesity: The state of the controversy. Crit Rev Clin Lab Sci 53, 52-67.
Tait Wojno, E. D., and Artis, D. (2016). Emerging concepts and future challenges in innate lymphoid cell biology. The Journal of experimental medicine 213, 2229-2248.
Tan, T. G., Sefik, E., Geva-Zatorsky, N., Kua, L., Naskar, D., Teng, F., Pasman, L., Ortiz-Lopez, A., Jupp, R., Wu, H. J., et al. (2016). Identifying species of symbiont bacteria from the human gut that, alone, can induce intestinal Th17 cells in mice. Proceedings of the National Academy of Sciences of the United States of America 113, E8141-E8150.
Tilg, H., Zmora, N., Adolph, T. E., and Elinav, E. (2020). The intestinal microbiota fueling metabolic inflammation. Nature reviews Immunology 20, 40-54.
Turnbaugh, P. J., Backhed, F., Fulton, L., and Gordon, J. I. (2008). Diet-induced obesity is linked to marked but reversible alterations in the mouse distal gut microbiome. Cell Host Microbe 3, 213-223.
Turnbaugh, P. J., Ley, R. E., Mahowald, M. A., Magrini, V., Mardis, E. R., and Gordon, J. I. (2006). An obesity-associated gut microbiome with increased capacity for energy harvest. Nature 444, 1027-1031.
Umesaki, Y., Okada, Y., Matsumoto, S., Imaoka, A., and Setoyama, H. (1995). Segmented filamentous bacteria are indigenous intestinal bacteria that activate intraepithelial lymphocytes and induce MHC class II molecules and fucosyl asialo GM1 glycolipids on the small intestinal epithelial cells in the ex-germ-free mouse. Microbiol Immunol 39, 555-562.
Upadhyay, V., Poroyko, V., Kim, T. J., Devkota, S., Fu, S., Liu, D., Tumanov, A. V., Koroleva, E. P., Deng, L., Nagler, C., et al. (2012). Lymphotoxin regulates commensal responses to enable diet-induced obesity. Nat Immunol 13, 947-953.
Vivier, E., Artis, D., Colonna, M., Diefenbach, A., Di Santo, J. P., Eberl, G., Koyasu, S., Locksley, R. M., Mckenzie, A. N. J., Mebius, R. E., et al. (2018). Innate Lymphoid Cells: 10 Years On. Cell 174, 1054-1066.
Wang, Q., Garrity, G. M., Tiedje, J. M., and Cole, J. R. (2007). Naive Bayesian classifier for rapid assignment of rRNA sequences into the new bacterial taxonomy. Applied and environmental microbiology 73, 5261-5267.
Wang, X., Ota, N., Manzanillo, P., Kates, L., Zavala-Solorio, J., Eidenschenk, C., Zhang, J., Lesch, J., Lee, W. P., Ross, J., et al. (2014). Interleukin-22 alleviates metabolic disorders and restores mucosal immunity in diabetes. Nature 514, 237-241.
Wang, Y., Kuang, Z., Yu, X., Ruhn, K. A., Kubo, M., and Hooper, L. V. (2017). The intestinal microbiota regulates body composition through NFIL3 and the circadian clock. Science 357, 912-916.
Ward, Z. J., Bleich, S. N., Cradock, A. L., Barrett, J. L., Giles, C. M., Flax, C., Long, M. W., and Gortmaker, S. L. (2019). Projected U. S. State-Level Prevalence of Adult Obesity and Severe Obesity. The New England journal of medicine 381, 2440-2450.
Winer, D. A., Luck, H., Tsai, S., and Winer, S. (2016). The Intestinal Immune System in Obesity and Insulin Resistance. Cell Metab 23, 413-426.
Wong, N., Fam, B. C., Cempako, G. R., Steinberg, G. R., Walder, K., Kay, T. W., Proietto, J., and Andrikopoulos, S. (2011). Deficiency in interferon-gamma results in reduced body weight and better glucose tolerance in mice. Endocrinology 152, 3690-3699.
Wu, L., Hollinshead, K. E. R., Hao, Y., Au, C., Kroehling, L., Ng, C., Lin, W. Y., Li, D., Silva, H. M., Shin, J., et al. (2020). Niche-Selective Inhibition of Pathogenic Th17 Cells by Targeting Metabolic Redundancy. Cell 182, 641-654 e620.
Yang, J., Park, K. W., and Cho, S. (2018). Inhibition of the CD36 receptor reduces visceral fat accumulation and improves insulin resistance in obese mice carrying the BDNF-Val66Met variant. The Journal of biological chemistry 293, 13338-13348.
Yang, Y., Torchinsky, M. B., Gobert, M., Xiong, H., Xu, M., Linehan, J. L., Alonzo, F., Ng, C., Chen, A., Lin, X., et al. (2014). Focused specificity of intestinal TH17 cells towards commensal bacterial antigens. Nature 510, 152-156.
Zagato, E., Pozzi, C., Bertocchi, A., Schioppa, T., Saccheri, F., Guglietta, S., Fosso, B., Melocchi, L., Nizzoli, G., Troisi, J., et al. (2020). Endogenous murine microbiota member Faecalibaculum rodentium and its human homologue protect from intestinal tumour growth. Nat Microbiol 5, 511-524.
Zhang, H., DiBaise, J. K., Zuccolo, A., Kudrna, D., Braidotti, M., Yu, Y., Parameswaran, P., Crowell, M. D., Wing, R., Rittmann, B. E., et al. (2009). Human gut microbiota in obesity and after gastric bypass. Proc Natl Acad Sci USA 106, 2365-2370.
Zmora, N., Bashiardes, S., Levy, M., and Elinav, E. (2017). The Role of the Immune System in Metabolic Health and Disease. Cell Metab 25, 506-521.
Zou, J., Chassaing, B., Singh, V., Pellizzon, M., Ricci, M., Fythe, M. D., Kumar, M. V., and Gewirtz, A. T. (2018). Fiber-Mediated Nourishment of Gut Microbiota Protects against Diet-Induced Obesity by Restoring IL-22-Mediated Colonic Health. Cell Host Microbe 23, 41-53.e44.

1. A method for treating or preventing one or more of metabolic syndrome, obesity, and associated morbidities in a subject suffering from or prone to metabolic syndrome, obesity, or any associated morbidity, the method comprising maintaining or increasing the levels of intestinal Th17 cells in the subject.

2. The method of claim 1, wherein the associated morbidity includes one or more of type-2 diabetes, cardiovascular disease, and non-alcoholic steatohepatitis or non-alcoholic fatty liver (NASH/NAFLD).

3. The method of claim 1, further comprising blocking type 3 innate lymphoid cells (ILC3) or IL-22 in the subject.

4. The method of claim 3, wherein the subject is administered a neutralizing antibody against ILC3 or IL-22.

5. The method of any one of claims 1-4, wherein the levels of intestinal Th17 cells are maintained or increased by administering to the subject an effective amount of commensal bacteria that induces production of Th17 cells.

6. The method of claim 5, wherein the commensal bacteria is a species selected from the group consisting of: Bifidobacterium, Eggerthella, Muribaculum, Olsenella, and Ruminococcus.

7. The method of claim 5, wherein the commensal bacteria comprise Bifidobacterium psudolongum.

8. The method of any one of claims 1-4, wherein the levels of intestinal Th17 cells are maintained or increased by administering to the subject an effective amount of commensal bacteria-induced Th17 cells.

9. The method of claim 8, wherein the commensal bacteria-induced Th17 cells are produced according to a method comprising:
isolating Th17 cells from the subject or a healthy donor;
expanding the isolated population of Th17 cells in vitro; and
administering the expanded isolated population of Th17 cells to the subject.

10. The method of claim 9, wherein the Th17 cells are isolated from the subject prior to subject receiving antibiotic treatment or undergoing any dietary interventions.

11. The method of any one of claims 1-4, wherein the levels of intestinal Th17 cells are maintained or increased by administering to the subject an antibiotic that preserves commensal Th17 cells.

12. The method of claim 11, wherein the antibiotics is selected from polymyxin B or streptomycin.

13. A method for treating or preventing one or more of metabolic syndrome, obesity, and associated morbidities in a subject suffering from or prone to metabolic syndrome, obesity, or any associated morbidity, the method comprising administering to the subject an effective amount of IL-17 or other intestinal Th17-cell derived molecules.

14. A method for treating or preventing one or more of metabolic syndrome, obesity, and associated morbidities in a subject suffering from or prone to metabolic syndrome, obesity, or any associated morbidity, the method comprising depleting Faecalibacterium rodentium or its homologue in the subject's intestinal microflora.

15. The method of claim 14, wherein the homologue is Holdemanella biformis.

16. A method for treating or preventing one or more of metabolic syndrome, obesity, and associated morbidities in a subject suffering from or prone to metabolic syndrome, obesity, or any associated morbidity, the method comprising depleting Erysipelotrichaceae in the subject's intestinal microflora.

17. A method for treating or preventing one or more of metabolic syndrome, obesity, and associated morbidities in a subject suffering from or prone to metabolic syndrome, obesity, or any associated morbidity, the method comprising a blockade of type 3 innate lymphoid cells (ILC3) or IL-22 in the subject.

18. The method of claim 17, wherein the subject is administered a neutralizing antibody against ILC3 or IL-22.

19. A method of manipulating dietary constraints or requirements in a subject based on levels of intestinal Th17 cells or Th17 cell function, e.g., IL-17.

20. A method of decreasing lipid absorption in a subject, the method comprising administering to the subject an effective amount of antagonists of intestinal CD36, NPC1L1, or SCD-1.
